SEQUENCE LISTING (110 sequences)

SEQ ID NO: 1 - DNA, 22 bp, synthetic construct
  misc_feature 1..22: note = Synthetic construct
  source 1..22: mol_type = other DNA
gacggccaggtcatcactattg

SEQ ID NO: 2 - DNA, 21 bp, synthetic construct
  misc_feature 1..21: note = Synthetic construct
  source 1..21: mol_type = other DNA
ctggtacatcagcacctcaca

SEQ ID NO: 3 - DNA, 21 bp, synthetic construct
  misc_feature 1..21: note = Synthetic construct
  source 1..21: mol_type = other DNA
ctctgggaaatcgtggaaatg

SEQ ID NO: 4 - DNA, 22 bp, synthetic construct
  misc_feature 1..22: note = Synthetic construct
  source 1..22: mol_type = other DNA
ccgaacgtgatccgctacttct

SEQ ID NO: 5 - DNA, 20 bp, synthetic construct
  misc_feature 1..20: note = Synthetic construct
  source 1..20: mol_type = other DNA
ggccaatcccaagagcagag

SEQ ID NO: 6 - DNA, 20 bp, synthetic construct
  misc_feature 1..20: note = Synthetic construct
  source 1..20: mol_type = other DNA
ctccactgctgcttcctgag

SEQ ID NO: 7 - DNA, 21 bp, synthetic construct
  misc_feature 1..21: note = Synthetic construct
  source 1..21: mol_type = other DNA
ccgtgttggttcatccctgta

SEQ ID NO: 8 - DNA, 17 bp, synthetic construct
  misc_feature 1..17: note = Synthetic construct
  source 1..17: mol_type = other DNA
tgacgtcgtggaactgc

SEQ ID NO: 9 - DNA, 22 bp, synthetic construct
  misc_feature 1..22: note = Synthetic construct
  source 1..22: mol_type = other DNA
gggacttcgctatcttcagaac

SEQ ID NO: 10 - DNA, 20 bp, synthetic construct
  misc_feature 1..20: note = Synthetic construct
  source 1..20: mol_type = other DNA
ggactctccacctgcaagac

SEQ ID NO: 11 - DNA, 21 bp, synthetic construct
  misc_feature 1..21: note = Synthetic construct
  source 1..21: mol_type = other DNA
ctgagtccgaatcaggtccag

SEQ ID NO: 12 - DNA, 21 bp, synthetic construct
  misc_feature 1..21: note = Synthetic construct
  source 1..21: mol_type = other DNA
cgcgacgtggaactggcagaa

SEQ ID NO: 13 - DNA, 20 bp, synthetic construct
  misc_feature 1..20: note = Synthetic construct
  source 1..20: mol_type = other DNA
gccctgagtcaagaggacag

SEQ ID NO: 14 - DNA, 20 bp, synthetic construct
  misc_feature 1..20: note = Synthetic construct
  source 1..20: mol_type = other DNA
caaggcttttgaaggcaaag

SEQ ID NO: 15 - DNA, 20 bp, synthetic construct
  misc_feature 1..20: note = Synthetic construct
  source 1..20: mol_type = other DNA
gtcacagatgtgccgaatgg

SEQ ID NO: 16 - DNA, 20 bp, synthetic construct
  misc_feature 1..20: note = Synthetic construct
  source 1..20: mol_type = other DNA
ggcggtggtgacagtatctt

SEQ ID NO: 17 - DNA, 22 bp, synthetic construct
  misc_feature 1..22: note = Synthetic construct
  source 1..22: mol_type = other DNA
ccacaggattccatacccaaga

SEQ ID NO: 18 - DNA, 20 bp, synthetic construct
  misc_feature 1..20: note = Synthetic construct
  source 1..20: mol_type = other DNA
gagctccttaacatgccctg

SEQ ID NO: 19 - DNA, 24 bp, synthetic construct
  misc_feature 1..24: note = Synthetic construct
  source 1..24: mol_type = other DNA
aagtgcatcatcgttgttcataca

SEQ ID NO: 20 - DNA, 22 bp, synthetic construct
  misc_feature 1..22: note = Synthetic construct
  source 1..22: mol_type = other DNA
cgcaaagtccttctgctccaca

SEQ ID NO: 21 - DNA, 20 bp, synthetic construct
  misc_feature 1..20: note = Synthetic construct
  source 1..20: mol_type = other DNA
gccacatgctagacacgcta

SEQ ID NO: 22 - DNA, 20 bp, synthetic construct
  misc_feature 1..20: note = Synthetic construct
  source 1..20: mol_type = other DNA
tgctgagctcatgccctttg

SEQ ID NO: 23 - DNA, 29 bp, synthetic construct
  misc_feature 1..29: note = Synthetic construct
  source 1..29: mol_type = other DNA
ttttggatttttaagacagagtctttgta

SEQ ID NO: 24 - DNA, 20 bp, synthetic construct
  misc_feature 1..20: note = Synthetic construct
  source 1..20: mol_type = other DNA
gaaagacttgtgaagtcggc

SEQ ID NO: 25 - DNA, 21 bp, synthetic construct
  misc_feature 1..21: note = Synthetic construct
  source 1..21: mol_type = other DNA
ggcgtgcttttaggaatgttg

SEQ ID NO: 26 - DNA, 20 bp, synthetic construct
  misc_feature 1..20: note = Synthetic construct
  source 1..20: mol_type = other DNA
catagatggcgttgttgcgg

SEQ ID NO: 27 - DNA, 21 bp, synthetic construct
  misc_feature 1..21: note = Synthetic construct
  source 1..21: mol_type = other DNA
gtccatgggaagatgttctgg

SEQ ID NO: 28 - DNA, 24 bp, synthetic construct
  misc_feature 1..24: note = Synthetic construct
  source 1..24: mol_type = other DNA
cttggtggtttgctacgacgtggg

SEQ ID NO: 29 - DNA, 20 bp, synthetic construct
  misc_feature 1..20: note = Synthetic construct
  source 1..20: mol_type = other DNA
ctcctaggcccctcagaagt

SEQ ID NO: 30 - DNA, 20 bp, synthetic construct
  misc_feature 1..20: note = Synthetic construct
  source 1..20: mol_type = other DNA
agaaggtgtttgtggctgct

SEQ ID NO: 31 - DNA, 20 bp, synthetic construct
  misc_feature 1..20: note = Synthetic construct
  source 1..20: mol_type = other DNA
tgagcgtgatcagctccagg

SEQ ID NO: 32 - DNA, 20 bp, synthetic construct
  misc_feature 1..20: note = Synthetic construct
  source 1..20: mol_type = other DNA
gtcactgacagaggcgatga

SEQ ID NO: 33 - DNA, 21 bp, synthetic construct
  misc_feature 1..21: note = Synthetic construct
  source 1..21: mol_type = other DNA
aaatccttgatattaaccagg

SEQ ID NO: 34 - DNA, 21 bp, synthetic construct
  misc_feature 1..21: note = Synthetic construct
  source 1..21: mol_type = other DNA
tttcccgtaaatatcacaccg

SEQ ID NO: 35 - DNA, 21 bp, synthetic construct
  misc_feature 1..21: note = Synthetic construct
  source 1..21: mol_type = other DNA
ttgaaactactttaagaacgg

SEQ ID NO: 36 - DNA, 21 bp, synthetic construct
  misc_feature 1..21: note = Synthetic construct
  source 1..21: mol_type = other DNA
aaattagcacattctttcagg

SEQ ID NO: 37 - DNA, 21 bp, synthetic construct
  misc_feature 1..21: note = Synthetic construct
  source 1..21: mol_type = other DNA
aaattgccaatataccaaggc

SEQ ID NO: 38 - DNA, 21 bp, synthetic construct
  misc_feature 1..21: note = Synthetic construct
  source 1..21: mol_type = other DNA
aatgagctgcatgaagtctcg

SEQ ID NO: 39 - DNA, 21 bp, synthetic construct
  misc_feature 1..21: note = Synthetic construct
  source 1..21: mol_type = other DNA
aaattggcacattctttcagg

SEQ ID NO: 40 - DNA, 21 bp, synthetic construct
  misc_feature 1..21: note = Synthetic construct
  source 1..21: mol_type = other DNA
ttcattaggaatacatcctgc

SEQ ID NO: 41 - DNA, 21 bp, synthetic construct
  misc_feature 1..21: note = Synthetic construct
  source 1..21: mol_type = other DNA
ttatttataggaaacactggg

SEQ ID NO: 42 - DNA, 19 bp, synthetic construct
  misc_feature 1..19: note = Synthetic construct
  source 1..19: mol_type = other DNA
aacgccacccatccaacca

SEQ ID NO: 43 - DNA, 22 bp, synthetic construct
  misc_feature 1..22: note = Synthetic construct
  source 1..22: mol_type = other DNA
gcaagctgaccctgaagttcat

SEQ ID NO: 44 - protein, 644 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFVPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSGIK

SEQ ID NO: 45 - protein, 609 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFVPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEGGTTVLATEKPTVIDSTIQSGIK

SEQ ID NO: 46 - protein, 704 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFVPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGAETIFIPLLYHFSSCLSWDQLTPVCVLVTPHGRELPRNRSCLARTRASSFPHVIWIDELFLIATTICNNNLSHFESQRLGLS

SEQ ID NO: 47 - protein, 923 aa, synthetic construct
  REGION 1..923: note = Synthetic construct
  VARIANT 11: note = X is any amino acid
  VARIANT 15: note = X is any amino acid
  VARIANT 18: note = X is any amino acid
  VARIANT 24: note = X is any amino acid
  VARIANT 29: note = X is any amino acid
  VARIANT 35: note = X is any amino acid
  VARIANT 62: note = X is any amino acid
  VARIANT 90: note = X is any amino acid
  VARIANT 96: note = X is any amino acid
  VARIANT 98: note = X is any amino acid
  VARIANT 99: note = X is any amino acid
  VARIANT 100: note = X is any amino acid
  VARIANT 100: note = X is any amino acid
  VARIANT 110: note = X is any amino acid
  VARIANT 140: note = X is any amino acid
  VARIANT 153: note = X is any amino acid
  VARIANT 155: note = X is any amino acid
  VARIANT 170: note = X is any amino acid
  VARIANT 170: note = X is any amino acid
  VARIANT 177: note = X is any amino acid
  VARIANT 196: note = X is any amino acid
  VARIANT 204: note = X is any amino acid
  VARIANT 219: note = X is any amino acid
  VARIANT 242: note = X is any amino acid
  VARIANT 269: note = X is any amino acid
  VARIANT 298: note = X is any amino acid
  VARIANT 303: note = X is any amino acid
  VARIANT 323: note = X is any amino acid
  VARIANT 359: note = X is any amino acid
  VARIANT 360: note = X is any amino acid
  VARIANT 362: note = X is any amino acid
  VARIANT 371: note = X is any amino acid
  VARIANT 372: note = X is any amino acid
  VARIANT 378: note = X is any amino acid
  VARIANT 379: note = X is any amino acid
  VARIANT 380: note = X is any amino acid
  VARIANT 392: note = X is any amino acid
  VARIANT 393: note = X is any amino acid
  VARIANT 393: note = X is any amino acid
  VARIANT 396: note = X is any amino acid
  VARIANT 409: note = X is any amino acid
  VARIANT 410: note = X is any amino acid
  VARIANT 449: note = X is any amino acid
  VARIANT 453: note = X is any amino acid
  VARIANT 469: note = X is any amino acid
  VARIANT 476: note = X is any amino acid
  VARIANT 479: note = X is any amino acid
  VARIANT 481: note = X is any amino acid
  VARIANT 487: note = X is any amino acid
  VARIANT 491: note = X is any amino acid
  VARIANT 498: note = X is any amino acid
  VARIANT 518: note = X is any amino acid
  VARIANT 518: note = X is any amino acid
  VARIANT 518: note = X is any amino acid
  VARIANT 528: note = X is any amino acid
  VARIANT 528: note = X is any amino acid
  VARIANT 553: note = X is any amino acid
  VARIANT 553: note = X is any amino acid
  VARIANT 555: note = X is any amino acid
  VARIANT 556: note = X is any amino acid
  VARIANT 572: note = X is any amino acid
  VARIANT 587: note = X is any amino acid
  VARIANT 599: note = X is any amino acid
  VARIANT 601: note = X is any amino acid
  VARIANT 634: note = X is any amino acid
  VARIANT 634: note = X is any amino acid
  VARIANT 667: note = X is any amino acid
  VARIANT 669: note = X is any amino acid
  VARIANT 672: note = X is any amino acid
  VARIANT 674: note = X is any amino acid
  VARIANT 717: note = X is any amino acid
  VARIANT 741: note = X is any amino acid
  VARIANT 755: note = X is any amino acid
  VARIANT 756: note = X is any amino acid
  VARIANT 805: note = X is any amino acid
  VARIANT 813: note = X is any amino acid
  VARIANT 820: note = X is any amino acid
  VARIANT 835: note = X is any amino acid
  VARIANT 838: note = X is any amino acid
  VARIANT 916: note = X is any amino acid
  VARIANT 919: note = X is any amino acid
MERGLPLLCAXLALXLAXAGAFRXDKCGXTIKIEXPGYLTSPGYPHSYHPSEKCEWLIQAPXPYQRIMINFNPHFDLEDRDCKYDYVEVXDGENEXGXXXGKFCGKIAPXPVVSSGPFLFIKFVSDYETHGAGFSIRYEXFKRGPECSQNYTXPXGVIKSPGFPEKYPNSLECTYIXFAPKMSEIILEFESFDLEXDSNPPGGXFCRYDRLEIWDGFPXVGPHIGRYCGQKTPGRIRSSSGXLSMVFYTDSAIAKEGFSANYSVLQSSXSEDFKCMEALGMESGEIHSDQITASSQYXTNWSXERSRLNYPENGWTPGEDSYXEWIQVDLGLLRFVTAVGTQGAISKETKKKYYVKTYXXDXSSNGEDWIXXKEGNKXXXFQGNTNPTDVVXXVFXKPLITRFVRIKPXXWETGISMRFEVYGCKITDYPCSGMLGMVSGLISDSQITXSNQXDRNWMPENIRLVTSRXGWALPPXPHXYXNEWLQXDLGXEKIVRGXIIQGGKHRENKVFMRKFKIXYSNNGSDWKXIMDDSKRKAKSFEGNNNYDTPELRXFXXLSTRFIRIYPERATHXGLGLRMELLGCEVEXPTAGPTTPNGNXVDECDDDQANCHSGTGDDFQLTGGTTVLATEKPTXIDSTIQSEFPTYGFNCEFGWGSHKTFCHWEHDXHXQLXWXVLTSKTGPIQDHTGDGNFIYSQADENQKGKVARLVSPVVYSQXSAHCMTFWYHMSGSHVGTLRVKLXYQKPEEYDQLVWMXXGHQGDHWKEGRVLLHKSLKLYQVIFEGEIGKGNLGGIAVDDISINNHIXQEDCAKPXLDKKNNXEIKIDETGSTPGYEXEGXGDKNISRKPGNVLKTLDPILITIIAMSALGVLLGAVCGVVLYCACWHNGMSERNLSALENYNFELVDGVKLKKDKLNXQSXYSEA

SEQ ID NO: 48 - protein, 923 aa, Mus musculus
MERGLPLLCATLALALALAGAFRSDKCGGTIKIENPGYLTSPGYPHSYHPSEKCEWLIQAPEPYQRIMINFNPHFDLEDRDCKYDYVEVIDGENEGGRLWGKFCGKIAPSPVVSSGPFLFIKFVSDYETHGAGFSIRYEIFKRGPECSQNYTAPTGVIKSPGFPEKYPNSLECTYIIFAPKMSEIILEFESFDLEQDSNPPGGMFCRYDRLEIWDGFPEVGPHIGRYCGQKTPGRIRSSSGVLSMVFYTDSAIAKEGFSANYSVLQSSISEDFKCMEALGMESGEIHSDQITASSQYGTNWSVERSRLNYPENGWTPGEDSYKEWIQVDLGLLRFVTAVGTQGAISKETKKKYYVKTYRVDISSNGEDWISLKEGNKAIIFQGNTNPTDVVLGVFSKPLITRFVRIKPVSWETGISMRFEVYGCKITDYPCSGMLGMVSGLISDSQITASNQADRNWMPENIRLVTSRTGWALPPSPHPYTNEWLQVDLGDEKIVRGVIIQGGKHRENKVFMRKFKIAYSNNGSDWKTIMDDSKRKAKSFEGNNNYDTPELRTFSPLSTRFIRIYPERATHSGLGLRMELLGCEVEAPTAGPTTPNGNPVDECDDDQANCHSGTGDDFQLTGGTTVLATEKPTIIDSTIQSEFPTYGFNCEFGWGSHKTFCHWEHDSHAQLRWSVLTSKTGPIQDHTGDGNFIYSQADENQKGKVARLVSPVVYSQSSAHCMTFWYHMSGSHVGTLRVKLRYQKPEEYDQLVWMVVGHQGDHWKEGRVLLHKSLKLYQVIFEGEIGKGNLGGIAVDDISINNHISQEDCAKPTDLDKKNTEIKIDETGSTPGYEGEGEGDKNISRKPGNVLKTLDPILITIIAMSALGVLLGAVCGVVLYCACWHNGMSERNLSALENYNFELVDGVKLKKDKLNPQSNYSEA

SEQ ID NO: 49 - protein, 922 aa, Rattus norvegicus
MERGLPLLCATLALALALAGAFRSDKCGGTIKIENPGYLTSPGYPHSYHPSEKCEWLIQAPEPYQRIMINFNPHFDLEDRDCKYDYVEVIDGENEGGRLWGKFCGKIAPSPVVSSGPFLFIKFVSDYETHGAGFSIRYEIFKRGPECSQNYTAPTGVIKSPGFPEKYPNSLECTYIIFAPKMSEIILEFESFDLEQDSNPPGGVFCRYDRLEIWDGFPEVGPHIGRYCGQKTPGRIRSSSGILSMVFYTDSAIAKEGFSANYSVLQSSISEDFKCMEALGMESGEIHSDQITASSQYGTNWSVERSRLNYPENGWTPGEDSYREWIQVDLGLLRFVTAVGTQGAISKETKKKYYVKTYRVDISSNGEDWITLKEGNKAIIFQGNTNPTDVVFGVFPKPLITRFVRIKPASWETGISMRFEVYGCKITDYPCSGMLGMVSGLISDSQITASNQGDRNWMPENIRLVTSRTGWALPPSPHPYINEWLQVDLGDEKIVRGVIIQGGKHRENKVFMRKFKIAYSNNGSDWKMIMDDSKRKAKSFEGNNNYDTPELRAFTPLSTRFIRIYPERATHSGLGLRMELLGCEVEVPTAGPTTPNGNPVDECDDDQANCHSGTGDDFQLTGGTTVLATEKPTIIDSTIQSEFPTYGFNCEFGWGSHKTFCHWEHDSHAQLRWRVLTSKTGPIQDHTGDGNFIYSQADENQKGKVARLVSPVVYSQSSAHCMTFWYHMSGSHVGTLRVKLHYQKPEEYDQLVWMVVGHQGDHWKEGRVLLHKSLKLYQVIFEGEIGKGNLGGIAVDDISINNHIPQEDCAKPTDLDKKNTEIKIDETGSTPGYEEGKGDKNISRKPGNVLKTLDPILITIIAMSALGVLLGAVCGVVLYCACWHNGMSERNLSALENYNFELVDGVKLKKDKLNPQSNYSEA

SEQ ID NO: 50 - protein, 771 aa, Homo sapiens [sequence omitted]

SEQ ID NO: 51 - DNA, 1308 bp, synthetic construct
  misc_feature 1..1308: note = Synthetic construct
  source 1..1308: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagagaagtcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcagatctggatccaaggaaaacttgtatttccagggc

SEQ ID NO: 52 - protein, 436 aa, synthetic construct
  REGION 1..436: note = Synthetic construct
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEKSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCRSGSKENLYFQG

SEQ ID NO: 53 - DNA, 1785 bp, synthetic construct
  misc_feature 1..1785: note = Synthetic construct
  source 1..1785: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagagaagtcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtagatctggatccaaggaaaacttgtatttccagggc

SEQ ID NO: 54 - protein, 595 aa, synthetic construct
  REGION 1..595: note = Synthetic construct
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEKSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCRSGSKENLYFQG

SEQ ID NO: 55 - DNA, 2154 bp, synthetic construct
  misc_feature 1..2154: note = Synthetic construct
  source 1..2154: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaaaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 56 - protein, 718 aa, synthetic construct
  REGION 1..718: note = Synthetic construct
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 57 - DNA, 1335 bp, synthetic construct
  misc_feature 1..1335: note = Synthetic construct
  source 1..1335: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaaaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtagatctggatccaaggaaaacttgtatttccagggc

SEQ ID NO: 58 - protein, 445 aa, synthetic construct
  REGION 1..445: note = Synthetic construct
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCRSGSKENLYFQG

SEQ ID NO: 59 - DNA, 2256 bp, synthetic construct
  misc_feature 1..2256: note = Synthetic construct
  source 1..2256: mol_type = other DNA
atggagagggggctgccgctcctctgcgccgtgctcgccctcgtcctcgccccggccggcgcttttcgcaacgataaaagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 60 - protein, 752 aa, synthetic construct
  REGION 1..752: note = Synthetic construct
MERGLPLLCAVLALVLAPAGAFRNDKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 61 - DNA, 1437 bp, synthetic construct
  misc_feature 1..1437: note = Synthetic construct
  source 1..1437: mol_type = other DNA
atggagagggggctgccgctcctctgcgccgtgctcgccctcgtcctcgccccggccggcgcttttcgcaacgataaaagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtagatctggatccaaggaaaacttgtatttccagggc

SEQ ID NO: 62 - protein, 479 aa, synthetic construct
  REGION 1..479: note = Synthetic construct
MERGLPLLCAVLALVLAPAGAFRNDKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCRSGSKENLYFQG

SEQ ID NO: 63 - DNA, 960 bp, synthetic construct
  misc_feature 1..960: note = Synthetic construct
  source 1..960: mol_type = other DNA
atggagagggggctgccgctcctctgcgccgtgctcgccctcgtcctcgccccggccggcgcttttcgcaacgataaaagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcagatctggatccaaggaaaacttgtatttccagggc

SEQ ID NO: 64 - protein, 320 aa, synthetic construct
  REGION 1..320: note = Synthetic construct
MERGLPLLCAVLALVLAPAGAFRNDKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCRSGSKENLYFQG

SEQ ID NO: 65 - DNA, 1797 bp, synthetic construct
  misc_feature 1..1797: note = Synthetic construct
  source 1..1797: mol_type = other DNA
atggagagggggctgccgctcctctgcgccgtgctcgccctcgtcctcgccccggccggcgcttttcgcaacgataaaagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatcctgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 66 - protein, 599 aa, synthetic construct
  REGION 1..599: note = Synthetic construct
MERGLPLLCAVLALVLAPAGAFRNDKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 67 - DNA, 2604 bp, synthetic construct
  misc_feature 1..2604: note = Synthetic construct
  source 1..2604: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 68 - protein, 847 aa, synthetic construct
  REGION 1..847: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 69 - DNA, 2604 bp, synthetic construct
  misc_feature 1..2604: note = Synthetic construct
  source 1..2604: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccaggctagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 70 - protein, 847 aa, synthetic construct
  REGION 1..847: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQASTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 71 - DNA, 2604 bp, synthetic construct
  misc_feature 1..2604: note = Synthetic construct
  source 1..2604: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggaaagaagtcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 72 - protein, 847 aa, synthetic construct
  REGION 1..847: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGKKSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 73 - DNA, 2604 bp, synthetic construct
  misc_feature 1..2604: note = Synthetic construct
  source 1..2604: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccattgccaaaaagaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 74 - protein, 847 aa, synthetic construct
  REGION 1..847: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAIAKKTKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 75 - DNA, 2604 bp, synthetic construct
  misc_feature 1..2604: note = Synthetic construct
  source 1..2604: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagagaagtcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 76 - protein, 847 aa, synthetic construct
  REGION 1..847: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEKSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 77 - DNA, 1785 bp, synthetic construct
  misc_feature 1..1785: note = Synthetic construct
  source 1..1785: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtagatctggatccaaggaaaacttgtatttccagggc

SEQ ID NO: 78 - protein, 574 aa, synthetic construct
  REGION 1..574: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCRSGSKENLYFQG

SEQ ID NO: 79 - DNA, 1785 bp, synthetic construct
  misc_feature 1..1785: note = Synthetic construct
  source 1..1785: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccaggctagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtagatctggatccaaggaaaacttgtatttccagggc

SEQ ID NO: 80 - protein, 574 aa, synthetic construct
  REGION 1..574: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQASTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCRSGSKENLYFQG

SEQ ID NO: 81 - DNA, 1308 bp, synthetic construct
  misc_feature 1..1308: note = Synthetic construct
  source 1..1308: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcagatctggatccaaggaaaacttgtatttccagggc

SEQ ID NO: 82 - protein, 415 aa, synthetic construct
  REGION 1..415: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCRSGSKENLYFQG

SEQ ID NO: 83 - DNA, 1308 bp, synthetic construct
  misc_feature 1..1308: note = Synthetic construct
  source 1..1308: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccaggctagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcagatctggatccaaggaaaacttgtatttccagggc

SEQ ID NO: 84 - protein, 415 aa, synthetic construct
  REGION 1..415: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQASTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCRSGSKENLYFQG

SEQ ID NO: 85 - DNA, 831 bp, synthetic construct
  misc_feature 1..831: note = Synthetic construct
  source 1..831: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgagatctggatccaaggaaaacttgtatttccagggc

SEQ ID NO: 86 - protein, 256 aa, synthetic construct
  REGION 1..256: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLRSGSKENLYFQG

SEQ ID NO: 87 - DNA, 2145 bp, synthetic construct
  misc_feature 1..2145: note = Synthetic construct
  source 1..2145: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatcctgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 88 - protein, 694 aa, synthetic construct
  REGION 1..694: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 89 - DNA, 2145 bp, synthetic construct
  misc_feature 1..2145: note = Synthetic construct
  source 1..2145: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccaggctagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatcctgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 90 - protein, 694 aa, synthetic construct
  REGION 1..694: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQASTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 91 - DNA, 1677 bp, synthetic construct
  misc_feature 1..1677: note = Synthetic construct
  source 1..1677: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaagaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatctggatccaaggaaaacttgtatttccagggc

SEQ ID NO: 92 - protein, 538 aa, synthetic construct
  REGION 1..538: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPRSGSKENLYFQG

SEQ ID NO: 93 - DNA, 2115 bp, synthetic construct
  misc_feature 1..2115: note = Synthetic construct
  source 1..2115: mol_type = other DNA
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaacttttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccctacagatgttgtggttgcagtattccccaaaccactgataactcgatttgtccgaatcaagcctgcaacttgggaaactggcatatctatgagatttgaagtatatggttgcaagataacagattatcctgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccagatct

SEQ ID NO: 94 - protein, 682 aa, synthetic construct
  REGION 1..682: note = Synthetic construct
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYELFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDP

SEQ ID NO: 95 - protein, 923 aa, synthetic construct
  REGION 1..923: note = Synthetic construct
MERGLPLLCATLALALALAGAFRSDKCGGTIKIENPGYLTSPGYPHSYHPSEKCEWLIQAPEPYQRIMINFNPHFDLEDRDCKYDYVEVIDGENEGGRLWGKFCGKIAPSPVVSSGPFLFIKFVSDYETHGAGFSIRYEIFKRGPECSQNYTAPTGVIKSPGFPEKYPNSLECTYIIFAPKMSEIILEFESFDLEQDSNPPGGVFCRYDRLEIWDGFPEVGPHIGRYCGQKTPGRIRSSSGILSMVFYTDSAIAKEGFSANYSVLQSSISEDFKCMEALGMESGEIHSDQITASSQYGTNWSVERSRLNYPENGWTPGEDSYREWIQVDLGLLRFVTAVGTQGAISKETKKKYYVKTYRVDISSNGEDWITLKEGNKAIIFQGNTNPTDVVFGVFPKPLITRFVRIKPASWETGISMRFEVYGCKITDYPCSGMLGMVSGLISDSQITASNQGDRNWMPENIRLVTSRTGWALPPSPHPYINEWLQVDLGDEKIVRGVIIQGGKHRENKVFMRKFKIAYSNNGSDWKMIMDDSKRKAKSFEGNNNYDTPELRAFTPLSTRFIRIYPERATHSGLGLRMELLGCEVEVPTAGPTTPNGNPVDECDDDQANCHSGTGDDFQLTGGTTVLATEKPTIIDSTIQSEFPTYGFNCEFGWGSHKTFCHWEHDSHAQLRWRVLTSKTGPIQDHTGDGNFIYSQADENQKGKVARLVSPVVYSQSSAHCMTFWYHMSGSHVGTLRVKLHYQKPEEYDQLVWMVVGHQGDHWKEGRVLLHKSLKLYQVIFEGEIGKGNLGGIAVDDISINNHIPQEDCAKPTDLDKKNTEIKIDETGSTPGYEEEGKGDKNISRKPGNVLKTLDPILITIIAMSALGVLLGAVCGVVLYCACWHNGMSERNLSALENYNFELVDGVKLKKDKLNPQSNYSEA

SEQ ID NO: 96 - protein, 923 aa, synthetic construct [sequence omitted]

SEQ ID NO: 97 - protein, 7 aa, synthetic construct
  REGION 1..7: note = Synthetic construct
  VARIANT 2: note = X can be any amino acid
  VARIANT 3: note = X can be any amino acid
  VARIANT 5: note = X can be any amino acid
EXXYXQG

SEQ ID NO: 98 - protein, 7 aa, synthetic construct
  REGION 1..7: note = Synthetic construct
  VARIANT 2: note = X is any amino acid
  VARIANT 3: note = X is any amino acid
  VARIANT 5: note = X is any amino acid
EXXYXQS

SEQ ID NO: 99 - DNA, 20 bp, synthetic construct
  misc_feature 1..20: note = NRP1 forward primer
  source 1..20: mol_type = other DNA
acccacatttcgatttggag

SEQ ID NO: 100 - DNA, 20 bp, synthetic construct
  misc_feature 1..20: note = NRP1 reverse primer
  source 1..20: mol_type = other DNA
ttcatagcggatggaaaacc

SEQ ID NO: 101 - DNA, 20 bp, synthetic construct
  misc_feature 1..20: note = SEMA3A forward primer
  source 1..20: mol_type = other DNA
gctcctgctccgtagcctgc

SEQ ID NO: 102 - DNA, 20 bp, synthetic construct
  misc_feature 1..20: note = SEMA3A Reverse primer
  source 1..20: mol_type = other DNA
tcggcgttgctttcggtccc

SEQ ID NO: 103 - DNA, 14 bp, synthetic construct
  misc_feature 1..14: note = SEMA3e forward primer
  source 1..14: mol_type = other DNA
tctgcaaccatcca

SEQ ID NO: 104 - DNA, 22 bp, synthetic construct
  misc_feature 1..22: note = SEMA3e reverse primer
  source 1..22: mol_type = other DNA
accacaagagggaagcacagac

SEQ ID NO: 105 - DNA, 20 bp, synthetic construct
  misc_feature 1..20: note = TGFb forward primer
  source 1..20: mol_type = other DNA
ggactctccacctgcaagac

SEQ ID NO: 106 - DNA, 20 bp, synthetic construct
  misc_feature 1..20: note = TGFb reverse primer
  source 1..20: mol_type = other DNA
catagatggcgttgttgcgg

SEQ ID NO: 107 - DNA, 20 bp, synthetic construct
  misc_feature 1..20: note = VEGFa forward primer
  source 1..20: mol_type = other DNA
gccctgagtcaagaggacag

SEQ ID NO: 108 - DNA, 20 bp, synthetic construct
  misc_feature 1..20: note = VEGFa reverse primer
  source 1..20: mol_type = other DNA
ctcctaggcccctcagaagt

SEQ ID NO: 109 - DNA, 20 bp, synthetic construct
  misc_feature 1..20: note = VEGFb forward primer
  source 1..20: mol_type = other DNA
tctgagcatggaactcatgg

SEQ ID NO: 110 - DNA, 20 bp, synthetic construct
  misc_feature 1..20: note = VEGFb reverse primer
  source 1..20: mol_type = other DNA
tctgcattcacattggctgt

(19) United States
(12) Patent Application Publication
(10) Pub. No.: US 2024/0301429 A1
(43) Pub. Date: Sep. 12, 2024

(54) COMPOSITIONS COMPRISING SASP MODULATORS AND SENESCENCE ATTENUATORS AND USES THEREOF FOR MODULATING CELLULAR SENESCENCE

(71) Applicant: RSEM, LIMITED PARTNERSHIP, QUEBEC (CA)

(72) Inventors: PRZEMYSLAW SAPIEHA, BEACONSFIELD (CA); FRÉDÉRICK ANTOINE MALLETTE, MONTREAL (CA); MALIKA OUBAHA, MONTREAL (CA); NORMAND BEAULIEU, MONTREAL (CA); ARIEL WILSON, MONTREAL (CA)

(21) Appl. No.: 18/675,928

(22) Filed: May 28, 2024

Related U.S. Application Data
(63) Continuation of application No. 17/302,113, filed on Apr. 23, 2021, now Pat. No. 12,031,137.
Application No. 17/302,113 is a continuation of application No. 16/335,896, filed on Mar. 22, 2019, now abandoned.
(60) Provisional application No. 62/398,183, filed on Sep. 22, 2016.
(60) Provisional application No. 62/398,797, filed on Sep. 23, 2016.
(60) Provisional application No. 62/398,819, filed on Sep. 23, 2016.

(51) Int. Cl. C12N 15/113; A61K 31/155; A61K 31/7088; A61K 38/17; A61P 3/04; C07K 16/28

(52) U.S. Cl. CPC C12N 15/1138; A61K 31/155; A61K 31/7088; A61K 38/179; A61P 3/04; C07K 16/2863; C07K 2319/30; C12N 2310/111; C12N 2310/122; C12N 2310/531

(57) ABSTRACT

Described herein are compositions and methods for modulating cellular senescence of a cell or induction of the senescence-associated secretory phenotype (SASP) in a cell. The methods generally comprise modulating the level or activity of IRE1a as a mean to control cellular senescence and induction of the SASP. Also described are methods for treating and preventing ocular vascular diseases comprising contacting cells in an eye of a subject with a biguanide compound and ophthalmic compositions comprising a biguanide compound.

### CROSS REFERENCE TO RELATED APPLICATIONS

This application is a continuation application of U.S. application Ser. No. 17/302,113 filed on Apr. 23, 2021, which is a continuation application of U.S. application Ser. No. 16/335,896 filed on Mar. 22, 2019, which is a national phase application of PCT application having Serial No PCT/CA2017/051120 filed on Sep. 22, 2017 and published in English under PCT Article 21(2), which itself claims benefit of U.S. provisional application Ser. No. 62/474,827, filed on Sep. 23, 2016, U.S. provisional application Ser. No. 62/398,797, filed on Sep. 23, 2016, and U.S. provisional application Ser. No. 62/398, 183 filed on Sep. 22, 2016. All of the above applications are incorporated herein by reference in their entirety.

### REFERENCE TO SEQUENCE LISTING

This application contains a Sequence Listing in computer readable form entitled “G16761-00050_Sequence Listing.xml”, created on May 20, 2024 and having a size of about 226000 bytes. The computer readable form is incorporated herein by reference.

### FIELD OF THE INVENTION

The present invention relates to compositions and methods for modulating cellular senescence. More specifically, the present invention is concerned with the modulation of the senescence-associated secretory phenotype (SASP) in the prevention and treatment of diseases and conditions associated with cellular senescence such as vascular ocular diseases.

### BACKGROUND OF THE INVENTION

Cellular senescence is commonly defined as a condition of a cell in which the cell remains viable and metabolically active but has lost the ability to proliferate. Cellular senescence may be caused by a variety of stimuli or factors including telomere shortening due to DNA end replication, DNA damage, altered activities of tumor suppressor genes and oncogenes, oxidative stress, inflammation, chemotherapeutic agents, and exposure to UV irradiation and ionizing radiation (Kuilman et al., Genes & Development. (2010) 24:2463-2479).

Three types of cellular paths leading to a senescence phenotype have been described: replicative senescence, premature senescence and senescence after differentiation (SAD). Replicative senescence is the type of senescence that occurs following a large number of cell division. For example, when grown in culture, primary cells undergo cellular senescence after approximately 50 cell divisions. This barrier to further proliferation is thought to be due to shortening of the cell's telomeres with each successive cell division, causing cells to reach a point (the so-called “Hayflick limit”) at which a DNA damage response is triggered, leading ultimately to induction of proliferation arrest and senescence.

Cellular senescence can also be induced in the absence of telomere loss or dysfunction. This type of cellular senescence is called premature cellular senescence and may result from a variety of stimuli including, for example, DNA damage arising from chemotherapy, radiotherapy, exposure to DNA damaging compounds or stimuli such as sunlight and UV light, oxidative stress, inflammation, strong mitogenic signaling and ribosomal stress. DNA damage may take the form of chromosomal dysfunction such as aneuploidy arising from unequal chromosome segregation during mitosis, DNA strand breaks, or chemical modification of DNA (e.g. alkylation). Premature cellular senescence may also be induced by a DNA damage response (DDR) which may or may not reflect actual DNA damage.

Recently, it has become apparent that the senescence process entails more than a simple cessation of cell growth as terminally differentiated, post-mitotic cells have been shown to acquire a senescence-like phenotype (including the SASP) in several diseases. This third type of senescence has been termed senescence after differentiation (SAD) and can be induced by various stressors including genotoxic, proteotoxic, oxidative and ribosomal stressors (see for example, Naylor R M et al., 2013 Clin. Pharmacol Ther. 93(1): 105-116).

Not all senescent cells express all possible senescence makers. Nonetheless, salient features of senescent cells include (i) growth arrest, (ii) enlarged and flatten cell morphology, (iii) DNA damage foci in the nucleus, (iv) secretion of growth factors proteases, cytokines and other factors defined as the senescence-associated secretory phenotypes (SASP), (v) senescence-associated-galactosidase (SA-β-gal) activity (which partly reflects the increase in lysosomal mass), (vi) expression of the tumor suppressor p16INK4a (which may activate pRB and cause the formation of senescence-associated heterochromatin foci (SAHF)), and (vii) increase in number and size of PML nuclear bodies. Furthermore, although diverse factors are known to induce cellular senescence, two tumor suppressor pathways, p53/p21 and p16INK4/pRB, have been shown to play a critical role in the regulation of cellular senescence.

Recent work has extended the involvement of cellular senescence to complex physiological processes such as embryogenesis and tissue repair (19-22). Conversely, in chronic diseases and aging, accretion of senescent cells aggravates tissue dysfunction (23-25). Depending on the condition, cellular senescence has been shown to be either beneficial or detrimental (see Rodier and Campisi, JCB, 2011, 192(4): 547-556 and Naylor et al., Clin Pharmacol Ther. 2013 93(1): 105-116 for review on cellular senescence).

Cellular senescence has been causally implicated in the pathogenesis of diverse age-related diseases and conditions including thinning of the epidermis, skin wrinkling, hair loss and greying hair, reduction in muscle thickness and muscle strength (sarcopenia), increased incidence of inflammation, metabolic disturbances, loss of endurance, atherosclerosis, chronic obstructive pulmonary disease (COPD), Idiopathic pulmonary fibrosis (IPF), neurodegenerative disease, osteoarthritis, osteoporosis, Parkinson's disease, and cataracts. In addition, cellular senescence is believed to contribute to damage to healthy tissues experienced during and following chemotherapy and/or radiotherapy, and the poor health effects post chemotherapy and/or radiotherapy.

Cellular senescence can also be beneficial. For example, its role as an anticancer mechanism in response to DNA damage has been established for decades. Furthermore, senescent cells have been shown to be important for efficient tissue repair and wound healing. Indeed, many factors of the SASP (e.g., growth factors and proteases that participate in wound healing, attractants for immune cells that kill pathogens and proteins that mobilize stem or progenitor cells) are important for tissue repair. The SASP may thus also serve to communicate cellular damage/dysfunction to the surrounding tissue and stimulate repair, if needed. Recent studies support this concept. For example, studies have shown that senescent cells are quickly established near wounds to help mount an inflammatory response (through the SASP) that initiates the process of healing during the proliferation phase. This rapid boost in senescence attracts and activates immune cells to fight infection and clear dead cells and debris. During the remodeling phase, senescent cells play a role in dissolving the fibrous proteins laid down during the proliferative phase and limit the formation of scars. Beneficial effects of cellular senescence have also been reported in liver fibrosis, myocardial infarction and cardiac fibrosis, atherosclerosis and pulmonary hypertension.

Accordingly, preventing cells from undergoing cellular senescence, or preventing DNA damage, DNA damage response pathways or chromatin changes that would activate senescence (e.g., that could lead to SASP), reversing or limiting cellular senescence and/or reducing paracrine senescence in cells which have undergone cellular senescence, would be advantageous to prevent or treat diseases and conditions in which senescence is detrimental. Conversely, promoting cellular senescence in diseases and conditions which are positively affected by cellular senescence may improve recovery or reduce the severity of such disease or conditions.

Obesity and its ensuing sequelae of metabolic syndrome, type 2 diabetes mellitus (TIIDM) and cardiovascular complications constitute a global pandemic. Worldwide obesity has more than doubled since 1980, and in 2014 more than 1.9 billion adults were overweight—of these 600 million were obese (World Health Organization (WHO), 2015).

Overweight and obesity are defined as abnormal or excessive fat accumulation that may impair health. Body mass index (BMI) is a simple index of weight-for-height that is commonly used to classify overweight and obesity in adults. It is defined as a person's weight in kilograms divided by the square of his height in meters (kg/m²). The WHO definition is: (i) a BMI greater than or equal to 25 kg/m² is overweight; and (ii) a BMI greater than or equal to 30 kg/m² is obesity. BMI provides a useful population-level measure of overweight and obesity as it is the same for both sexes and for all ages of adults. However, it is considered a rough guide because it may not correspond to the same degree of fatness in different individuals.

Fat accumulation is observed in a range of conditions such as obesity, non-alcoholic fatty liver disease (NAFLD), metabolic syndrome and lipodystrophy syndrome. Elevated BMI (in excessive weight or obesity) is a major risk factor for diseases and conditions such as: cardiovascular diseases ((CVD), mainly heart diseases and stroke); and insulin resistance (which increases the risk of developing TIIDM). Excessive fat accumulation also increases the risk of suffering from other diseases or conditions including musculoskeletal disorders (especially osteoarthritis); and some cancers (endometrial, breast, and colon). The risk for these diseases generally increases, with an increase in BMI.

Metabolic syndrome, also known as syndrome X, affects persons with obesity as well as those with an increased amount of abdominal fat, and is characterized by insulin resistance, dyslipidemia (hypertriglyceridemia, low serum HDL cholesterol levels, and increased LDL cholesterol levels) and hypertension. These conditions are interrelated and share underlying mediators, mechanisms and pathways. Changes in fat distribution, increased waist to hip ratio (WHR) and central fat accumulation are related to increased metabolic risk indices.

Most of the conditions associated with metabolic syndrome have no symptoms, although a large waist circumference is a visible sign. Several organizations have criteria for diagnosing metabolic syndrome. The NCEP ATP III definition is one of the most widely used criteria of metabolic syndrome. It incorporates the key features of hyperglycemia/insulin resistance, visceral obesity, atherogenic dyslipidemia and hypertension/endothelial dysfunction. According to guidelines used by the National Institutes of Health, a subject has metabolic syndrome if three or more of the following traits are present or if the subject is taking medication to control them: (i) Visceral obesity (i.e., large waist circumference—for example, a waistline that measures at least 35 inches (89 centimeters) for women and 40 inches (102 centimeters) for men); (ii) High triglyceride level—150 milligrams per deciliter (mg/dL), or 1.7 millimoles per liter (mmol/L), or higher of this type of fat found in blood; (iii) Reduced high-density lipoprotein (HDL) cholesterol-less than 40 mg/dl (1.04 mmol/L) in men or less than 50 mg/dl (1.3 mmol/L) in women of this “good” cholesterol; (iv) Increased blood pressure—130/85 millimeters of mercury (mm Hg) or higher; and (v) Elevated fasting blood sugar—100 mg/dl (5.6 mmol/L) or higher.

The currently accepted mechanism of obesity induced-metabolic syndrome is that adipose lipid accumulation triggers cytokine release, inducing M1 activation and systemic inflammation (Olefsky and Glass, 2010). While chronic inflammation and macrophage activation is postulated to cause insulin resistance (Osborn and Olefsky, 2012), it remains controversial whether adipose tissue inflammation is an adaptive response permitting effective storage of excess nutrients (Wernstedt Asterholm et al., 2014) and whether proper angiogenesis is a prerequisite for adipose tissue expansion (Cullberg et al., 2013). Both adipocyte oxygen consumption (Lee et al., 2014) and adipose tissue vascular remodeling (Sung et al., 2013) controls the inflammatory state of adipose tissue, which subsequently lead to insulin insensitivity and hyperglycemia.

Despite increasing social awareness regarding obesity-related problems, the proportion of overweight and obese subjects continues to rise. Thus, in view of their high prevalence and associated morbidity and mortality, there remains a need to develop new approaches for the prevention and/or treatment of diseases and conditions associated with fat accumulation.

The present description refers to a number of documents, the content of which is herein incorporated by reference in their entirety.

### SUMMARY OF THE INVENTION

The breakdown of vascular beds in ischemic retinopathies, whether it is glycemia-driven in diabetic retinopathy (DR) or oxygen-driven in retinopathy of prematurity (ROP), yields hypoxic/ischemic central nervous system (CNS) tissue subjected to a collection of biochemical and inflammatory stressors that compromise cellular function (1-3). These avascular zones are the source of pro-angiogenic factors that mediate pathological angiogenesis (4) as evidenced by clinical success of laser photocoagulation therapy that ablate these areas (5). While much effort has been invested in understanding the ensuing wave of pathological pre-retinal angiogenesis, relatively little is known of the cellular processes at play during the precursory state of neural tissue hypoxia. A more thorough understanding of the cellular responses operating during the initial stages of retinal ischemia may provide therapeutic avenues that benefit the portion of the 93 million individuals faced with DR and the 15 million preterm infants born each year that present with neovascular retinal disease (6-8).

Central neurons such as retinal ganglion cells (RGCs), which are directly apposed to degenerating vasculature in ischemic retinopathies, require stable metabolic supply for proper function. Interestingly, during progression of DR there is a disconnect between the extent of overt retinal vascular lesions (9) and the relatively subtle and protracted morphological and functional aberrations observed in RGCs (10-12). Furthermore, while there is evidence supporting RGC apoptosis in DR (13-15) the magnitude and dynamics of neuronal death remain a topic of debate (16-18). The relative resilience of retinal ganglion neurons in DR suggests they either receive metabolic supply from an alternative vascular plexus or initiate a protective mechanism that renders them less susceptible to ischemia-induced cell death.

Mechanisms triggered to preserve nervous tissue integrity during ischemic injury confer a critical survival advantage and allow for timely repair and restoration of function (44, 61, 62). Mechanisms leading to cellular senescence likely evolved parallel to those of apoptosis to limit oncogenesis (21), yet for post-mitotic CNS neurons such as those found in the retina, cellular senescence may prevent stressor-induced neurodegeneration. While studied for close to 60 years (63) in the context of aging and disease, Applicant's studies reported herein present a novel role for cellular senescence in weathering ischemia in the CNS. These studies further revealed a previously undescribed role for SEMA3A in modulating senescence, including paracrine senescence in pathological processes and uncover the therapeutic benefits of modulating SEMA3A activity in diseases and conditions associated with senescence.

More specifically, in an aspect, Applicants have identified an unsuspected mechanism triggered by neurons in devascularized retinal zones where they enter a state of premature cellular senescence and adopt a senescence-associated secretory phenotype (SASP) by activating the endoribonuclease activity of the ER-stress effector Inositol-Requiring Enzyme-1α (IRE-1α). Factors produced through the SASP including the secreted embryonic patterning cue Semaphorin3A (SEMA3A), propagate senescence across the ischemic retina to neurons, microglia and the overlying vasculature (paracrine senescence), contributing to destructive pre-retinal angiogenesis.

### 1. Compositions Comprising SASP Modulators and Senescence Attenuators and Uses Thereof for Treating and Preventing Ocular Diseases

In a first aspect, data described herein show that pathways of senescence are initially engaged in the retina as a mechanism of homeostasis in order to weather hypoxic stress. However, when persistent, senescence pathways become pathological and compromise tissue integrity. A consequence of cellular senescence is the SASP that through secretion of inflammatory factors hinders adequate revascularization. Notably, as shown herein, analysis of patients suffering from proliferative diabetic retinopathy, showed SASP-associated cytokines in their vitreous. Furthermore, pharmacological inhibition of the SASP with the well-known biguanide metformin or pharmacological or genetic interference against IRE1α limits senescence, enhances reparative vascular regeneration, prevents destructive neovascularization (FIG. 6H-J), and stalls retinal pathology in vivo. These data provide evidence for a previously undescribed paradigm implicating cellular and paracrine senescence in pathological angiogenesis and uncover the therapeutic benefits of ocular delivery of modulators of the SASP to treat ocular vascular diseases (vasculopathies) such as retinopathies as well as Age-related macular degeneration (AMD) and macular edema.

Thus, in accordance with the present invention, there is provided a method of treating or preventing a vascular eye disease or disorder (an ocular vasculopathy, in particular a senescence-associated eye disease or disorder, e.g., a retinopathy) comprising reducing (attenuating/inhibiting) cellular senescence in the eye of the subject. Reduction of cellular senescence can be made by contacting cells of the subject with one or more compounds which reduce cellular senescence (a senescence inhibitor). In embodiments, the senescence inhibitor reduces or inhibits the SASP in ocular cells.

The present invention further provides a method of inhibiting retinal angiogenesis (pathological neovascularization) comprising administering a senescence inhibitor (e.g., a SASP inhibitor) to a subject. In embodiments, the retinal angiogenesis is secondary to ischemia.

The present invention also provides a method of promoting ocular vascular repair and/or reducing ocular ischemia comprising administering a senescence inhibitor (e.g., a SASP inhibitor) to a subject.

The present invention also provides a method of preventing or reducing ocular cellular senescence comprising administering a senescence inhibitor a subject. In embodiments, the senescence inhibitor is a SASP inhibitor.

The present invention also provides a method of preventing or reducing ocular cellular senescence comprising contacting an ocular cell with a senescence inhibitor. In embodiments, the senescence inhibitor is a SASP inhibitor.

In embodiments, the above-noted senescence is paracrine senescence. In embodiments, the senescence is senescence after differentiation. In embodiments, the senescence is premature senescence. In embodiments, the premature senescence in characterized by an increase in the expression and/or RNAse activity of IRE1α. In embodiments, the senescence is retinal senescence. In embodiments, the senescence is characterized by (i) increased expression and/or activity of P16INK4a, Tp53, IRE1a, Cdkn1a Cdkn2a and/or senescence associated beta-gal activity; (ii) expression of γH2Ax and/or PML; and/or (iii) the expression of the senescence-associated secretory phenotype (SASP). In embodiments, the SASP comprises the secretion of IL-1β, IL-6, Pai1, TGFβ1, IRE1a and/or VEGF-.a. In embodiments, the above-mentioned SASP is secondary to cellular ischemia.

In embodiments, the cell is a human cell. In embodiments, the cell is a retinal cell. In embodiments, the cell is an endothelial cell. In embodiments, the cell is a microglial cell. In embodiments, the cell is a neuron. In embodiments, the cell is a retinal ganglion cell. In embodiments, the cell is a retinal ganglion neuron. In embodiments, the cell is a vascular cell. In embodiments, the cell is a vascular endothelial cell. In embodiments, the cell is not a vascular cell (i.e., it is located in an avascular area/region). In embodiments, the cell is a fibroblast. In embodiments, the cell is a macrophage.

In embodiments, the administration is topical or local ocular administration. In embodiments, the local ocular administration is subconjunctival (sub-tenons), intravitreal, retrobulbar, posterior juxtascleral or intracameral administration. In embodiments, the local ocular administration is intravitreal administration. In particular embodiments, the local ocular administration is an intravitreal injection.

The present invention also relates to a composition comprising a senescence inhibitor or a SASP inhibitor for use in the methods of the present invention. In embodiments, the composition is an ophthalmic composition. In embodiments, the composition comprises a suitable pharmaceutical carrier, diluent or excipient. In embodiments, the suitable pharmaceutical carrier, diluent or excipient is not normally found in mixtures with the inhibitors disclosed herein (i.e., is a non-naturally occurring carrier or the composition is not naturally found in nature, i.e., is synthetic or manmade). In embodiments, the composition is for treating or preventing a vascular eye disease or disorder. In embodiments, the composition is for inhibiting retinal angiogenesis. In embodiments, the composition is for promoting ocular vascular repair and/or reducing ocular ischemia. In embodiments, the composition is for preventing or reducing ocular cellular senescence. In embodiments, the composition is for use in the preparation of a medicament for (i) treating or preventing a vascular eye disease or disorder; (ii) inhibiting retinal angiogenesis (e.g., pathological retinal neovascularization); (iii) promoting ocular vascular repair and/or reducing ocular ischemia; and/or (iv) preventing or reducing ocular cellular senescence.

In embodiments, the above-mentioned SASP inhibitor is not an inhibitor of IRE1α. In embodiments, the SASP inhibitor is a biguanide compound. In embodiments, the biguanide compound is metformin, phenformin, buformin, proguanil, chlorproguanil, Synthalin A or Synthalin B. In embodiments, the biguanide compound is metformin. In embodiments, the SASP inhibitor is an inhibitor of IRE1α.

In embodiments, the vascular eye disease or disorder is diabetic retinopathy, retinopathy of prematurity, ischemic retinopathy, hypertensive retinopathy, drug-induced retinal vasculopathy, diabetic macular edema, age-related macular degeneration, juvenile macular degeneration, retinal neovascularisation, central retinal vein occlusion, branched retinal vein occlusion, choroidal neovascularization, polypoidal choroidal vasculopathy, physical injury to the eye, glaucoma, rhegmatogenous retinal detachment (RRD), retinal vasculitis, retinal macroaneurysm, retinal microaneurysm, Fuch's dystrophy, ischemic optic neuropathy, macular telangiectasia, optic neuritis, usher syndrome, retinitis pigmentosa, uveitis, ischemic optic neuropathy (ION) or Stangardt disease. In an embodiment, the vascular eye disease or disorder is diabetic retinopathy, retinopathy of prematurity, diabetic macular edema, age-related macular edema, retinal neovascularization, central retinal vein occlusion, branched retinal vein occlusion or choroidal neovascularization. In an embodiment, the vascular eye disease is diabetic retinopathy, retinopathy of prematurity, Dry (atrophic) Age-related Macular Degeneration, wet (exudative) Age-related Macular Degeneration, Branch Retinal Vein Occlusion, or Macular Talacgiectasia.

In embodiments, the subject treated with a SASP inhibitor or composition of the present invention has been diagnosed with one of the above-noted vascular eye disease or disorder. In embodiments, the subject has been diagnosed with diabetic retinopathy, retinopathy of prematurity, diabetic macular edema, age-related macular degeneration, pathological retinal neovascularisation, central retinal vein occlusion, branched retinal vein occlusion, choroidal neovascularization, polypoidal choroidal vasculopathy or Macular Talacgiectasia.

### 2. Compositions and Methods for Modulating Cellular Senescence Comprising IRE1α or SEMA3A Modulators

### (i) IRE1α Modulators

The present invention also provides a method of inhibiting or preventing (i) cellular senescence of a cell or (ii) the senescence-associated secretory phenotype (and/or the induction thereof) of a cell comprising reducing IRE1α expression, activation or activity. The present invention also concerns a method of inhibiting or preventing (i) cellular senescence of a cell or (ii) induction of the senescence-associated secretory phenotype in a cell of a subject comprising administering to the subject an IRE1a inhibitor. In embodiments, the methods of the present invention reduce IRE1α activation; SA-B-gal activity; and/or the expression of Pai1, IL-6, II-1b, TGF-b, tp53, XBP1(s) and/or Vegfa in the cells.

The present invention further concerns a composition comprising an IRE1α inhibitor for (i) inhibiting or preventing cellular senescence of a cell or (ii) induction of the senescence-associated secretory phenotype in a cell comprising an IRE1α inhibitor. In embodiments, the composition is for (i) inhibiting or preventing cellular senescence of a cell or (ii) induction of the senescence-associated secretory phenotype in a cell. In embodiments, the composition is for use in the preparation of a medicament for (i) inhibiting or preventing cellular senescence of a cell or (ii) induction of the senescence-associated secretory phenotype in a cell.

In embodiments, the IRE1α inhibitor is: an antisens or shRNA against IRE1α, 4u8c, bortezomib, N-[(2-Hydroxy-1-naphthalenyl)methylene]-2-thiophenesulfonamide (STF-083010), or MKC-3946. In embodiments, the inhibitor reduces IRE1α activation; SA-β-gal activity; and/or the expression of Pai1, IL-6, II-1b, TGF-b, tp53, XBP1(s) and/or Vegfa in the cells.

The present invention also provides a method of stimulating or inducing (i) senescence of a cell or (ii) the senescence-associated secretory phenotype (SASP) of a cell (and/or the induction thereof) comprising increasing IRE1α level or activity. The present invention also provides a method of improving wound repair comprising increasing IRE1α level or activity, wherein the method increases or induces (i) cellular senescence of a cell or (ii) the senescence-associated secretory phenotype (SASP) in a cell. The present invention further provides a method of stimulating or inducing (i) cellular senescence of a cell or (ii) the senescence-associated secretory phenotype in a cell of a subject comprising increasing IRE1α level or activity. In embodiments, the above-noted methods comprise contacting the cell with a compound which increases IRE1α level or activity. In embodiments, the above-mentioned methods increase IRE1α activation; SA-β-gal activity; and/or the expression of Pai1, IL-6, II-1b, TGF-b, tp53, XBP1(s) and/or Vegfa in the cells.

In embodiments, the IRE1a activity comprises IRE1a ribonuclease activity and kinase activity.

The present invention further provides a composition comprising a compound which increases IRE1α level or activity. In embodiments, the composition is for inducing (i) senescence of a cell or (ii) the senescence-associated secretory phenotype in a cell. In embodiments, the composition is for treating or preventing liver fibrosis, renal fibrosis, pulmonary hypertension, myocardial infarction or cardiac fibrosis. In embodiments, the composition is for improving wound healing. In embodiments, the composition is for use in the preparation of a medicament for inducing (i) senescence of a cell or (ii) the senescence-associated secretory phenotype in a cell. In embodiments, the composition is for use in the preparation of a medicament for treating or preventing liver fibrosis, renal fibrosis, pulmonary hypertension, myocardial infarction or cardiac fibrosis. In embodiments, the composition is for use in the preparation of a medicament for improving wound healing.

In embodiments, the compound which increases IRE1α level or activity is Apy29 or Sunitinib.

In embodiments, the above-noted cell is a terminally differentiated cell. In embodiments, the cell is a neuron, a microglial cell or an endothelial cell. In embodiments, the cell is a retinal cell. In embodiments, the cell is a myeloid cell. In embodiments, the cell is a retinal ganglion cell. In embodiments, the cell is a retinal ganglion neuron. In embodiments, the cell is a vascular cell. In embodiments, the cell is a vascular endothelial cell. In embodiments, the cell is not a vascular cell (i.e., it is located in an avascular area/region). In embodiments, the cell is a fibroblast. In embodiments, the cell is a macrophage. In embodiments, the cell is a monocyte. In embodiments, the cell is an hepatic cell. In embodiments, the cell is an hepatic stellate cell. In embodiments, the cell is a human cell. In embodiments, the cell is a human microvascular endothelial cell. In particular embodiments, the cell is not an ocular cell.

In embodiments, the above-mentioned senescence is paracrine senescence. In embodiments, the senescence is senescence after differentiation. In embodiments, the senescence is premature senescence. In embodiments, the premature senescence in characterized by an increase in the expression and/or RNAse activity of IRE1a. In embodiments, the senescence is retinal senescence. In embodiments, the senescence is characterized by (i) increased expression and/or activity of P16INK4a, Tp53, IRE1a, Cdkn1a Cdkn2a and/or senescence associated beta-gal activity; (ii) expression of γH2Ax and/or PML; and/or (iii) the expression of the senescence-associated secretory phenotype (SASP). In embodiments, the SASP comprises the secretion of IL-1β, IL-6, Pai1, TGFβ1, IRE1a and/or VEGFa. In embodiments, the above-mentioned SASP is secondary to cellular ischemia. In embodiments, the cell is from a subject suffering or at risk of suffering from sarcopenia, neurodegeneration, thinning of the epidermis, skin wrinkling, hair loss, chronic obstructive pulmonary disease (COPD), Idiopathic pulmonary fibrosis (IPF), atherosclerosis, osteoarthritis, osteoporosis or Parkinson's disease, intestinal bowel disease, glaucoma, intervertebral disc degeneration, brain aneurysm, aortic aneurysm, pancreatic fibrosis or cystic fibrosis. In embodiments, the cell is from a subject which has undergone cancer treatment or is undergoing cancer treatment. In embodiments, the cell is not a retinal cell. In embodiments, the cellular senescence is not associated with a retinal vascular disease (i.e., it does not arise in the context of a retinal disease). In embodiments, the cellular senescence is not associated with a vascular eye disease (i.e., it does not arise in the context of an eye disease). In embodiments, the cellular senescence is not associated diabetic retinopathy (i.e., it does not arise in the context of diabetic retinopathy). In embodiments, the cellular senescence is not associated with macular degeneration. In embodiments, the cell is from a subject having or at risk of having, liver fibrosis, renal fibrosis, pulmonary hypertension, myocardial infarction or cardiac fibrosis. In embodiments, the subject is wounded (e.g., has a cutaneous/tissue wound (e.g., cut).

### (ii) SEMA3A Modulators

In a further aspect, data presented herein provide evidence for a previously undescribed role for SEMA3A in modulating senescence, including paracrine senescence in pathological processes and uncover the therapeutic benefits of modulating SEMA3A activity in diseases and conditions associated with senescence. Indeed, the data demonstrates that SEMA3A activates the ER-stress effector Inositol-Requiring Enzyme-1α (IRE-1α) and senescence effectors p53 and p16.

Accordingly, in an aspect, the present invention provides a method of modulating (i) senescence of a cell or (ii) the senescence-associated secretory phenotype (SASP) of a cell (and/or the induction thereof) comprising modulating SEMA3A level or activity. In embodiments, modulating SEMA3A level or activity comprises contacting the cell with a SEMA3A antagonist or a SEMA3A agonist.

In a related aspect, the present invention provides a method of inhibiting or preventing (i) senescence of a cell or (ii) the senescence-associated secretory phenotype (SASP) of a cell (and/or the induction thereof) comprising reducing SEMA3A level or activity. In embodiments, reducing SEMA3A level or activity comprises contacting the cell with a SEMA3A antagonist.

The present invention further provides a method of inhibiting or preventing (i) senescence of a cell or (ii) the senescence-associated secretory phenotype of a cell of a subject (and/or the induction thereof) comprising reducing SEMA3A level or activity. In embodiments, reducing SEMA3A level or activity comprises administering to the subject (or contacting the cells of the subject with) a SEMA3A antagonist.

In a further aspect, the present invention concerns a SEMA3A antagonist. In embodiments, the SEMA3A antagonist is (a) a SEMA3A antibody; (b) a SEMA3A antisense or shRNA; and/or (c) a soluble NRP1 polypeptide or functional fragment thereof (NRP1 trap).

The present invention also concerns a composition comprising the above-mentioned SEMA3A antagonist. Such antagonist or composition comprising same may be used in the above-described methods (e.g for use in inhibiting or preventing (i) senescence of a cell or (ii) induction of the senescence-associated secretory phenotype (SASP) of a cell).

In a related aspect, the present invention concerns the use of the SEMA3A antagonist or composition of the present invention in the preparation of a medicament for inhibiting or preventing (i) senescence of a cell or (ii) the senescence-associated secretory phenotype (SASP) in a cell (and/or the induction thereof). In embodiments, the methods and compositions described herein are for treating or preventing a senescence associated disease or condition which is sarcopenia, neurodegeneration (e.g., Alzheimer's disease), thinning of the epidermis, skin wrinkling, hair loss, chronic obstructive pulmonary disease (COPD), Idiopathic pulmonary fibrosis (IPF), atherosclerosis, osteoarthritis, osteoporosis, Parkinson's disease, intestinal bowel disease, glaucoma, intervertebral disc degeneration, brain aneurysm, aortic aneurysm, pancreatic fibrosis or cystic fibrosis, metabolic syndrome and/or obesity. In embodiments, the cell is not a retinal cell. In embodiments, the cell is not a retinal ganglion cell. In embodiments, the cell is not from an eye of a subject (ocular cell). In embodiments, the cellular senescence is not associated with a retinal vascular disease.

In embodiments, the above methods reduce IRE1a activation and the expression of Pai1, IL-6, II-1b, TGF-b, tp53, XBP1(s) and Vegfa in the cell.

In embodiments, the cell is a terminally differentiated cell. In embodiments, the cell is a neuron, a microglial cell or an endothelial cell. In embodiments, cell is a retinal cell. In embodiments, the cell is a myeloid cell. In embodiments, the cell is a fat tissue cell. In embodiments, the cell is a retinal ganglion cell. In embodiments, the cell is a retinal ganglion neuron. In embodiments, the cell is a vascular cell. In embodiments, the cell is a vascular endothelial cell. In embodiments, the cell is not a vascular cell (i.e., it is located in an avascular area/region). In embodiments, the cell is a fibroblast. In embodiments, the cell is a macrophage. In embodiments, the cell is a monocyte. In embodiments, the cell is an hepatic cell. In embodiments, the cell is an hepatic stellate cell. In embodiments, the cell is a human cell. In embodiments, the cell is a human microvascular endothelial cell. In particular embodiments, the cell is not an ocular cell. In embodiments, the above-mentioned senescence is paracrine senescence. In embodiments the senescence is secondary to cellular ischemia. In embodiments the SASP is secondary to cellular ischemia. In embodiments the senescence is secondary to glucose intolerance. In embodiments the SASP is secondary to glucose intolerance.

In embodiments, the cell is from a subject suffering or at risk of suffering from a senescence associated disease or condition which is sarcopenia, neurodegeneration (e.g., Alzheimer's disease), thinning of the epidermis, skin wrinkling, hair loss, chronic obstructive pulmonary disease (COPD), Idiopathic pulmonary fibrosis (IPF), atherosclerosis, osteoarthritis, osteoporosis, Parkinson's disease, intestinal bowel disease, glaucoma, intervertebral disc degeneration, brain aneurysm, aortic aneurysm, pancreatic fibrosis or cystic fibrosis, metabolic syndrome and/or obesity. In embodiments, the cell is not a retinal cell. In embodiments, the cell is not a retinal ganglion cell. In embodiments, the cell is not from an eye of a subject (ocular cell). In embodiments, the cellular senescence is not associated with a retinal vascular disease. In embodiments, the cellular senescence is not associated with a disease of the eye (ocular cellular senescence). In embodiments, the cellular senescence is not associated with Alzheimer's disease. In embodiments, the cellular senescence is not associated with diabetes. In embodiments, the cellular senescence is not associated with cancer. In embodiments, the cellular senescence is not associated with septic shock.

In another aspect, the present invention concerns a method of stimulating or inducing (i) senescence of a cell or (ii) the senescence-associated secretory phenotype of a cell comprising contacting said cell with a SEMA3A polypeptide or functional variant or fragment thereof.

Also provided is a method of stimulating or inducing (i) cellular senescence of a cell or (ii) the senescence-associated secretory phenotype in a cell of a subject comprising administering to said subject an effective amount of a SEMA3A polypeptide or functional variant or fragment thereof.

In a further aspect, the present invention concerns a method for improving wound healing in a tissue comprising cells, the method comprising contacting the cells with a SEMA3A polypeptide or functional variant or fragment thereof.

In a related aspect, the present invention provides a SEMA3A polypeptide or functional variant or fragment or variant thereof, nucleic acid encoding same, vector for delivering and/or expressing the SEMA3A polypeptide or functional variant or fragment and host cell comprising such polypeptide or functional variant or fragment, nucleic acid and/or vector.

The present invention also concerns compositions comprising the above-mentioned SEMA3A polypeptide or functional variant or fragment thereof, nucleic acid, vector and/or host cell. Such compositions, SEMA3A polypeptide or functional variant or fragment, nucleic acid, vector and host cell may be used in the above-described methods (e.g., (a) for inducing (i) senescence of a cell or (ii) the senescence associated secretory phenotype in a cell, (b) in the preparation of a medicament for inducing (i) senescence of a cell or (ii) the senescence associated secretory phenotype in a cell, or (c) for improving wound healing).

In embodiments, the above-mentioned cell in methods of stimulating or inducing (i) cellular senescence, (ii) the SASP, or (iii) wound healing is a terminally differentiated cell. In embodiments, the cell is a neuron, a microglial cell or an endothelial cell. In embodiments, the above-mentioned senescence is paracrine senescence. In embodiments, the SASP is secondary to cellular ischemia. In embodiments, the cell is from a subject having or at risk of having, liver fibrosis, pulmonary hypertension, myocardial infarction, cancer, renal fibrosis or cardiac fibrosis.

In embodiments, the above-mentioned methods increase IRE1a activation and the expression of Pai1, IL-6, II-1b, TGF-b, tp53, XBP1(s) and Vegfa in cells.

The present invention also provides nucleic acids encoding polypeptides (e.g., NRP1 traps, SEMA3A, IRE1α etc.) antisense, shRNAs etc. disclosed herein as well as vectors and host cells for delivering and/or expressing the nucleic acids, polypeptides, antisense, shRNAs disclosed herein.

Other objects, advantages and features of the present invention will become more apparent upon reading of the following non-restrictive description of specific embodiments thereof, given by way of example only with reference to the accompanying drawings.

### 3. Modulation of Lipid Parameters

In another aspect, the present invention concerns a method of altering a lipid parameter in a subject, said method comprising administering to the subject: (a) a soluble NRP1 polypeptide or fragment thereof; (b) an NRP1 antibody; or (c) a composition comprising (a) and/or (b) together with a pharmaceutically acceptable carrier,

wherein said altering of a lipid parameter is (a) a decrease in total cholesterol level; (b) a decrease in non-HDL cholesterol level; (c) a decrease in triglycerides level; (d) a decrease in the ratio of total cholesterol:HDL cholesterol; (e) a decrease in circulating free fatty acid; (f) an increase in HDL cholesterol or (f) any combination of (a) to (e).

In another aspect, the present invention concerns a method for preventing or treating a disease or condition associated with fat accumulation in a subject, said method comprising administering to the subject: (a) a soluble NRP1 polypeptide or fragment thereof; (b) an NRP1 antibody; or (c) a composition comprising (a) and/or (b) together with a pharmaceutically acceptable carrier.

In an embodiment, said disease or condition associated with fat accumulation is high body mass index (BMI); obesity; metabolic syndrome; NAFLD; a cardiovascular disease (CVD); hypertension and/or Type II Diabetes mellitus (TIIDM).

In an embodiment, said cardiovascular disease is congestive heart failure, hypercholesterolemia and/or atherosclerosis.

In another aspect, the present invention concerns a method for altering a body composition parameter in a subject comprising administering to the subject (a) a soluble NRP1 polypeptide or fragment thereof; (b) an NRP1 antibody; or (c) a composition comprising (a) and/or (b) together with a pharmaceutically acceptable carrier,

wherein said body composition parameter is visceral fat area (VFA), body mass index (BMI), waist to hip ratio (WHR); waist-to-height ratio (WHeR), waist circumference (WC); arm circumference (AC), conicity index, per cent body fat (PBF), triceps skin fold, subscapular skin fold, white adipose tissue (WAT) level; and or brown adipose (BAT) tissue level.

In embodiments, said soluble NRP1 polypeptide or fragment thereof comprises or consists of an NRP1 polypeptide trap described in Table 2 or set forth in FIG. 7 or 9A.

In an embodiment, said soluble NRP1 polypeptide or fragment thereof is administered systemically.

In another aspect, the present invention concerns a composition comprising (a) a soluble NRP1 polypeptide or fragment thereof; (b) an NRP1 antibody; or (c) a composition comprising (a) and/or (b), together with a pharmaceutically acceptable carrier, for altering a lipid parameter in a subject,

wherein said alteration of a lipid parameter is (a) a decrease in total cholesterol level; (b) a decrease in non-HDL cholesterol level; (c) a decrease in triglycerides level; (d) a decrease in the ratio of total cholesterol:HDL cholesterol; (e) a decrease in circulating free fatty acid; (f) an increase in HDL cholesterol or (f) any combination of (a) to (e).

In another aspect, the present invention concerns a composition comprising (a) a soluble NRP1 polypeptide or fragment thereof; (b) an NRP1 antibody; or (c) a composition comprising (a) and/or (b), together with a pharmaceutically acceptable carrier, for preventing or treating a disease or condition associated with fat accumulation in a subject.

In an embodiment, said disease or condition associated with fat accumulation is high body mass index (BMI); obesity; metabolic syndrome; NAFLD; a cardiovascular disease (CVD); hypertension and/or Type II Diabetes mellitus (TIIDM).

In an embodiment, said cardiovascular disease is congestive heart failure, hypercholesterolemia and/or atherosclerosis.

In another aspect, the present invention concerns a composition comprising (a) a soluble NRP1 polypeptide or fragment thereof; (b) an NRP1 antibody; or (c) a composition comprising (a) and/or (b), together with a pharmaceutically acceptable carrier for altering a body composition parameter in a subject,

wherein said body composition parameter is visceral fat area (VFA), body mass index (BMI), waist to hip ratio (WHR); waist-to-height ratio (WHeR), waist circumference (WC); arm circumference (AC), conicity index, per cent body fat (PBF), triceps skin fold, subscapular skin fold, white adipose tissue (WAT) level; and or brown adipose (BAT) tissue level.

In an embodiment, said soluble NRP1 polypeptide or fragment thereof comprises or consists of an NRP1 polypeptide trap described in Table 2 or set forth in FIG. 7 or 9A.

In an embodiment, said soluble NRP1 polypeptide or fragment thereof is for systemic administration.

In another aspect, the present invention concerns a use of (a) a soluble NRP1 polypeptide or fragment thereof; (b) an NRP1 antibody; or (c) a composition comprising (a) and/or (b) together with a pharmaceutically acceptable carrier; for altering a lipid parameter in a subject,

wherein said alteration of a lipid parameter is (a) a decrease in total cholesterol level; (b) a decrease in non-HDL cholesterol level; (c) a decrease in triglycerides level; (d) a decrease in the ratio of total cholesterol: HDL cholesterol; (e) a decrease in circulating free fatty acid; (f) an increase in HDL cholesterol or (f) any combination of (a) to (e).

In another aspect, the present invention concerns a use of a soluble NRP1 polypeptide or fragment thereof; (b) an NRP1 antibody; or (c) a composition comprising (a) and/or (b), together with a pharmaceutically acceptable carrier, for preventing or treating a disease or condition associated with fat accumulation in a subject.

In an embodiment, said disease or condition associated with fat accumulation is high body mass index (BMI); obesity; metabolic syndrome; NAFLD; a cardiovascular disease (CVD); hypertension and/or Type II Diabetes mellitus (TIIDM).

In an embodiment, said cardiovascular disease is congestive heart failure, hypercholesterolemia and/or atherosclerosis.

In another aspect, the present invention concerns a use of (a) a soluble NRP1 polypeptide or fragment thereof; (b) an NRP1 antibody; or (c) a composition comprising (a) and/or (b), together with a pharmaceutically acceptable carrier for altering a body composition parameter in a subject,

wherein said body composition parameter is visceral fat area (VFA), body mass index (BMI), waist to hip ratio (WHR); waist-to-height ratio (WHeR), waist circumference (WC); arm circumference (AC), conicity index, per cent body fat (PBF), triceps skin fold, subscapular skin fold, white adipose tissue (WAT) level; and or brown adipose (BAT) tissue level.

In an embodiment, said soluble NRP1 polypeptide or fragment thereof comprises or consists of an NRP1 polypeptide trap described in Table 2 or set forth in FIG. 7 or 9A.

In an embodiment, said soluble NRP1 polypeptide or fragment thereof is for systemic administration.

In another aspect, the present invention concerns a use of (a) a soluble NRP1 polypeptide or fragment thereof; (b) an NRP1 antibody; or (c) a composition comprising (a) and/or (b) together with a pharmaceutically acceptable carrier; for the preparation of a medicament for altering a lipid parameter in a subject,

wherein said altering of a lipid parameter is (a) a decrease in total cholesterol level; (b) a decrease in non-HDL cholesterol level; (c) a decrease in triglycerides level; (d) a decrease in the ratio of total cholesterol:HDL cholesterol; (e) a decrease in circulating free fatty acid; (f) an increase in HDL cholesterol or (f) any combination of (a) to (e).

In another aspect, the present invention concerns a use of a soluble NRP1 polypeptide or fragment thereof; (b) an NRP1 antibody; or (c) a composition comprising (a) and/or (b), together with a pharmaceutically acceptable carrier, for the preparation of a medicament for preventing or treating a disease or condition associated with fat accumulation in a subject.

In an embodiment, said disease or condition associated with fat accumulation is high body mass index (BMI); obesity; metabolic syndrome; NAFLD; a cardiovascular disease (CVD); hypertension and/or Type II Diabetes mellitus (TIIDM).

In an embodiment, said cardiovascular disease is congestive heart failure, hypercholesterolemia and/or atherosclerosis.

In another aspect, the present invention concerns a use of (a) a soluble NRP1 polypeptide or fragment thereof; (b) an NRP1 antibody; or (c) a composition comprising (a) and/or (b), together with a pharmaceutically acceptable carrier for the preparation of a medicament for altering a body composition parameter in a subject,

wherein said body composition parameter is visceral fat area (VFA), body mass index (BMI), waist to hip ratio (WHR); waist-to-height ratio (WHeR), waist circumference (WC); arm circumference (AC), conicity index, per cent body fat (PBF), triceps skin fold, subscapular skin fold, white adipose tissue (WAT) level; and or brown adipose (BAT) tissue level.

In an embodiment, said soluble NRP1 polypeptide or fragment thereof comprises or consists of an NRP1 polypeptide trap described in Table 2 or set forth in FIG. 7 or 9A.

In an embodiment, said medicament is for systemic administration.

### BRIEF DESCRIPTION OF THE DRAWINGS

In the appended drawings:

FIGS. 1A-J show that retinal ischemia triggers cellular senescence and a senescence-associated secretory phenotype. FIG. 1A: Schematic depiction of the mouse model of oxygen-induced retinopathy (OIR). FIG. 1B: Representative gene set enrichment analysis (GSEA) corresponding to signatures of inflammation and apoptosis from large-scale genome-wide RNA-seq of P14 normoxic and OIR retinas. Gene expression profiles positively and negatively correlated with OIR phenotype are represented. FIG. 1C: Heat map and GSEA cluster of Fridman senescence-associated genes (28) in P14 OIR (left colums) vs normoxic retinas (right colums). (NES=normalized enrichment score, FDRq=false discovery rate), color scale depicting expression of log2 (fold change) from low (−2, left) to high expression (2, right). FIG. 1D: Immunoblots of retinal cell lysates from P14 OIR and normoxic mice show induction of markers of senescence. FIG. 1E: RT-qPCR shows induction of expression of senescence-associated genes, Cdkn1a (p21) and Cdkn2a (p16) in P14 OIR vs normoxic retinas. FIG. 1F: Representative isolectin B4 (IB4) and senescence-associated B-galactosidase staining (SA-β-gal) of P14 normoxia and OIR flatmount retinas. FIG. 1G: Quantification of percentage of senescence-associated β-galactosidase staining (SA-β-gal) in P14 OIR vs normoxia flatmount retinas. Higher magnification views of boxed central (c)/peripheral (p) and vascular/avascular zones of retinas are shown (***P<0.0001 central compared with peripheral OIR retinas (n=10); †††P<0.0001 avascular compared to central P14 Normoxia retinas (n=7). FIG. 1H: Representative IB4 and SA-β-gal staining of sagittal section of P14 normoxia and OIR retinas. Higher magnification images of boxed central avascular retinal zones are shown in right panel. Retinal ganglion cells (GCL), inner nuclear layer (INL) and outer nuclear layer (ONL) are shown for orientation. FIG. 1I: Quantification of percent area stained with SA-β-gal in retinal sagittal sections at P14 (*P<0.05 compared to normoxic retinas (n=10), †P<0.05 compared to other P14 OIR layers of the retinas (n=7); ns=not significant). FIG. 1J: TUNEL staining of P14 OIR and normoxic retinas. Scale bars are 500 μm (FIG. 1H). For higher magnification images and (FIG. 1H, J) scale bars are 200 μm. Data are presented as mean±SEM.

FIGS. 2A-F show that cellular senescence propagates during progression of retinopathy. FIG. 2A: Isolectin B4 (IB4) staining of retinas during progression of OIR (P14, P17 and P21). FIG. 2B: Schematic illustration of propagation of cellular senescence throughout OIR depicting the SA-β-gal stained flatmount OIR retinas in lower panels (scale bars 50 μm). Higher magnification images reveal distinct senescent cell population (scale bars 25 μm). FIG. 2C: Representative confocal micrographs of P14 OIR retinas show robust staining of markers of senescence (γH2AX (top row) and Pai1 (middle row) in RGCs (Brn3a staining (2nd column). Insets are high magnification images of outlined areas. FIG. 2D: Representative confocal micrographs of P17 OIR flatmount retinas reveal that senescence markers (PML (bottom row) and γH2AX (top row) co-label with microglia (IBA1) and vessels IB4. FIG. 2E: Heat map and GSEA cluster of paracrine senescence-associated genes in P14 OIR vs normoxic retinas for two different samples. FIG. 2F: RT-qPCRs show induction of expression of Pai1, Il1β, Tgf-β1, Il6, Vegf-a, Ire1α and Tp53 in P14 OIR vs normoxic retinas. β-actin was used as a reference gene. Scale bars are 100 μm for FIG. 2C and 2D. Data are presented as mean±SEM.

FIGS. 3A-Q show that SEMA3A mediates senescence and paracrine senescence. FIG. 3A: Western blot analysis of SEMA3A protein expression level during OIR compared to normoxic controls at P10, P14, P17 and P21. β-actin is used as a loading control. FIG. 3B: Immunoblot analysis of SEMA3A protein levels during Ras-induced senescence in Mouse Embryonic Fibroblasts (MEFs). Cell lysates from MEFs retrovirally transduced with H-RasV12 oncogene or control empty vector harvested 14 days post-selection. FIG. 3C: SEMA3A transcript levels in human normal diploid fibroblasts (IMR90) retrovirally transduced with empty vector, MEK1 alone or MEK and human papillomavirus oncoproteins E6 and E7. Data are from GEO profile GSE2487 (77) (**P<0.05 CT compared to MEK and ††P<0.05 MEK compared to MEK/E6/E7). FIG. 3D: SEMA3A transcript levels in proliferating or senescent cultured primary human activated hepatic stellate cells (HSC) (**P=0.027). Data are from GEO profile GSE11954 (78). FIG. 3E: Retina from P5 pups that received intravitreal injection of recombinant SEMA3A (100 ng/ml) at P2, and harvested at P5, were subjected to immunoblot analysis against senescence markers and SA-β-gal staining of sagittal cryosections (FIG. 3F). FIG. 3G: Cell cycle distribution profiles of SEMA3A-treated (100 and 500 ng/ml) HRMECs (human retinal microvascular endothelial cells) obtained by FACS analysis 7 days after treatment (P<0.0001; Two-way ANOVA) FIG. 3H: Trans endothelial resistance measured in real time by electric cell impedance sensing (ECIS) demonstrated that SEMA3A reduced endothelial cell proliferation (3-7 days) (0.048>P>0.009). FIG. 3I: HRMEC, retinal neurons (661 W) and J774 (macrophages-like cells) stimulated with recombinant SEMA3A (100 ng/ml) or vehicle (CT) for 7 days, stained for SA-β-gal and quantification in FIG. 3J. (n=3 separate experiments), **P<0.005 and ***P<0.0001 from two-tailed Student's t test J, FIG. 3K: Conditioned medium (CM) was collected from senescent retinal neurons (661 W) and macrophage-like, J774 cells grown for 7 days after H₂O₂ stimulation (150 μM, 2 h). Senescent cells were stained with SA-β-gal (right) and the level of secreted SEMA3A (S3A) protein in CM was evaluated by immunoblot (left). FIG. 3L: SA-β-gal staining of neuronal 661 W cells infected with the indicated vectors (Lv.sh_GFP or Lv.sh_SEMA3A) and treated with H₂O₂ or vehicle (CT). Senescence was evaluated after 7 days of treatment. FIG. 3M: Quantification of percentage of SA-β-gal positive cells treated as in FIG. 3J. (n=3 independent experiments). *P<0.005, H₂O₂-treated sh_GFP cells compared to untreated sh_GFP cells; and †P<0.005, H₂O₂-treated sh_S3A cells compared to H₂O₂-treated sh_GFP cells, from two-tailed Student's t-test. Data are presented as mean±SEM. FIG. 3N: Induction of paracrine senescence (CM from senescent or not neuron precursor cells ((661 W) see FIG (I)) in HRMEC was evaluated after 7 days by SA-β-gal staining. FIG. 30: Quantification of percentage of SA-β-gal positive cells treated as in (I) with CM from the senescent 661 W cells. FIG. 3P: IB4 and SA-β-gal staining of retinas from P14 OIR mice injected intravitreally at P12 with Lv.sh_S3A or Lv.sh_GFP and quantified in FIG. 3Q (***P<0.0001).

FIGS. 4-C show Heatmap and GSEA for signatures of the unfolded protein response in P14 OIR vs normoxic retinas. FIG. 4A: Gene expression profiles positively and negatively correlated with OIR phenotype are represented. FIG. 4B: Representative SA-β-gal and IB4 stains of LysM-Cre/ROSA26EYFP^{fl/fl} retinas at P14 and P17 OIR. FIG. 4C: Representative γH2AX staining and EYFP in central regions from LysM-Cre/ROSA26EYFP^{fl/fl} at P14 and P17 OIR. White arrowheads point to co-labeling of γH2AX and EYFP⁺ microglia;

FIGS. 5A-H show that SEMA3A is secreted by senescent cells and triggers paracrine senescence in human retinal microvasculature endothelial cells. Relative mRNA levels of SEMA3A in P14 (FIG. 5A) and P17, P21 (FIG. 5B) retinas (normoxia vs OIR) measured by RT-qPCR. FIG. 5C: Western blot analysis of SEMA3A protein expression showing the efficiency of the Sh-RNA downregulation in neuronal cells (661 W). FIG. 5D: Schematic illustration explaining conditioned media (CM) experiments to tease out the contribution of paracrine senescence. CM from the retinal neuron precursor cell line expressing sh_GFP or sh_SEMA3A made senescent with H₂O₂, and CM was harvested and applied to HRMEC. Induction of paracrine senescence in HRMEC was evaluated after 7 days by SA-β-gal staining. FIG. 5E: Levels of secreted SEMA3A in CM from retinal neuron precursor cell infected with Lv.sh_GFP or Lv.sh_S3A were evaluated by immunoblot. FIG. 5F: Immunoblot analysis of p53 expression in HRMEC exposed to CM from senescent (or not) retinal neuron precursor cells (H₂O₂ or vehicle treated). FIG. 5G: Immunoblot analysis of HRMEC exposed to CM from retinal neuron precursor cells transfected with sh_GFP or sh_SEMA3A (sh_S3A) and treated with H₂O₂ or vehicle. FIG. 5H: Immunoblot analysis of ER-stress effector, p-IRE1α^{S724}, in HRMECs lysates treated with recombinant SEMA3A (100 ng/ml) or CM from senescent J774 cells (Sen-J774) and harvested after 7 days;

FIGS. 6A-F show that SEMA3A induces senescence in J774 macrophage/monocytes. FIG. 6A: P14 Retinal lysates from mice having received an intravitreal injection of Lv.sh_GFP or Lv.sh-IRE1α were immunoblotted against anti-IRE1α and Cre. FIG. 6B: SA-β-gal staining of J774 macrophages treated with SEMA3A (100 ng/ml, 7 days) or H₂O₂ as a positive control. Higher magnification views of the boxed regions are shown. FIG. 6C: Immunoblot analysis of J774 macrophages exposed to SEMA3A (100 ng/ml, 7 days) or H₂O₂, as positive control, and HRMEC treated with conditioned media (CM) from respective J774 conditions as indicated by dashed arrows. FIG. 6D: Immunoblot against IRE1α demonstrates efficiency of lentivirus-mediated depletion of IRE1α in HRMECs. FIG. 6E: SA-β-gal staining of HRMEC infected with indicated lentiviral vectors (Lv.sh_GFP and Lv.sh_IRE1α) and then treated with recombinant SEMA3A (100 ng/ml, 7 days) or vehicle (CT). FIG. 6F: Relative mRNA expression levels of Cdkn1a, Cdkn2a in J774 treated with SEMA3A (100 ng/ml, 7 days) or vehicle (CT);

FIGS. 7A-F show that the RNAse activity of IRE1α contributes to senescence. FIG. 7A: Immunoblots for markers of senescence in J774 cell lysates stimulated with SEMA3A (S3A) (100 ng/mL) or vehicle for 3 or 7 days shows induction of p-IRE1α^{S724}, p53, Pai1, (n=3). FIG. 7B: RT-qPCR in J774 macrophages revealed mRNA increased expression of Pai1, Il6, Il1β, Tgf-β1 and Tp53 after exposure to SEMA3A (100 ng/ml) for 7 days. FIG. 7C: Representative confocal immunofluorescence staining of γH2AX and DAPI of J774 macrophages stimulated with SEMA3A (100 ng/ml) or vehicle for 7 days (Scale bars are 100 μm). Higher magnification views of the outlined areas are shown (Scale bars are 50 μm). Immunoblots FIG. 7D: for the XBP1(s) (spliced isoform) and PCR (FIG. 7E) for XBP1(s) and unspliced XBP1 (u) in J774 cell lysates treated with vehicle, S3A (100 ng/ml) and /or 4μ8c (1 ng/ml) at day 7 post-treatment. FIG. 7F: RT-qPCR for levels of Pai1, Il6, Il1β, Tgf-β1, Tp53, Ire1α and Tnfα in J774 stimulated with SEMA3A alone or with 4μ8c. β-actin was used as a reference gene. ***P<0.0001, *P<0.005. Scale bars: 100 μm for FIGS. 7C & 7G. High mag C is 50 μm. Data are presented as mean±SEM;

FIGS. 8A-K show that Metformin abrogates the SASP and pathological retinal angiogenesis. FIG. 8A: Angiography, spectral domain optical coherence tomography (SD-OCT) and 3D retinal maps obtained from patients selected for the study. Control patients (CT) (n=10) with nonvascular ocular pathologies were compared with patients with proliferative diabetic retinopathy (PDR) (n=10) patients. Table 1 (Example 6) shows patients characteristics. FIG. 8B: Multiplex assessment of patient vitreous humor for cytokines involved in paracrine senescence shows induction in VEGF-A, Pai1, IL-6, and IL-8. Results are expressed as fold change normalized to CT patients. Points represent individual values; **P<0.001, ***P<0.0001. FIG. 8C: Immunoblot analysis for p-NFκB^{S536} and p-IRE1α^{S724} in retinal lysates from P14 and P17 of OIR mice intravitreally injected with metformin or vehicle at P12. FIG. 8D: RT-qPCR for levels of Cdkn1a, Cdkn2a and 116 measured in retinas of P14 OIR mice intravitreally injected with metformin or vehicle at P12 (β-actin was used as a reference gene). Representative P14 (FIG. 8E) and P17 (FIG. 8F) OIR flatmount retinas labeled with IB4 and SA-β-gal in mice intravitreally injected with metformin or vehicle at P12. FIG. 8G: Quantification of percentage SA-β-gal stained area in P14 and P17 OIR mice treated as in E and F. (**P=0.0042 at P14, (n=9); **P=0.0013 at P17 (n=11); metformin compared with vehicle-injected retinas). FIG. 8H: Representative IB4 stained flatmount retinas of P14 and P17 OIR mice intravitreally injected with metformin or vehicle at P12. Quantification of avascular areas at P14 (FIG. 8I) and P17 (FIG. 8J) of OIR. Pre-retinal neovascularization was assessed at P17 OIR FIG. 8K: Results are expressed as percentage of avascular or neovascular area versus the whole retinal area (***P<0.0001 and ***P<0.001; metformin compared to vehicle-injected retinas (n=13)). Horizontal bars represent mean value of percentage, and dots represent individual values. Scale bars are 500 μm. Data are presented as mean±SEM;

FIGS. 9A-G show that Aflibercept abrogates pathological angiogenesis without promoting vascular repair or cellular senescence. Representative P14 (FIG. 9A) and P17 (FIG. 9C) OIR flatmount retinas labeled with IB4 and SA-β-gal of mice intravitreally injected with Aflibercept or vehicle at P12. Quantification of percentage SA-β-gal stained area in P14 (FIG. 9B) and P17 (FIG. 9D) OIR mice treated as in A or C. (P=0.3087 at P14, (n=13-14); P=0.1580 at P17 (n=13); Aflibercept compared with vehicle-injected retinas). Quantification of avascular areas at P14 (P=0.4897, n=11-13) (FIG. 9E) and P17 (P=0.9502, n=6-7) (FIG. 9F) of OIR. Pre-retinal neovascularization was assessed at P17 OIR (FIG. 9G). Results are expressed as percentage of avascular or neovascular area versus the whole retinal area (*P=0.0207, n=5-6); Aflibercept compared to vehicle-injected retinas n=5-6). Horizontal bars represent mean value of percentage, and dots represent individual values. Scale bars are 500 μm. Data are presented as mean±SEM;

FIGS. 10A-F show that induction of retinal cellular senescence during OIR and in an STZ model of type I diabetes. FIG. 10A: Isolectin B4 (IB4) and TUNEL staining of the whole eye cryosections at P14 OIR and normoxia presented in FIG. 1I. FIG. 10B: Representative confocal immunofluorescence of γH2AX (green; left column), p-IRE1α^{S724} (green; middle column) and Pai1 (green; right column), isolectin B4 (IB4) (red), and DAPI (blue) on cryosectionned P14 normoxia and OIR retinas. FIG. 10C: Representative confocal immunofluorescence of PML (green; left column), p16 (green; right column), IB4 and DAPI of cryosectionned P14 OIR eyes. FIG. 10D: Representative confocal immunofluorescence against PML, isolectin B4 (IB4) and DAPI of flatmount retina at P21 OIR. FIG. 10E: Representative SA-β-gal staining of cryosections from citrate and STZ retinas. The retinal ganglion cells (GCL), inner nuclear layer (INL) and outer nuclear layer (ONL) are shown for orientation. FIG. 10F: Representative confocal immunofluorescence of α-SMA or NG2, isolectin B4 (IB4), on flatmount retinas from adult mice citrate (control) or STZ (diabetic);

FIGS. 11A-B show vascular coverage during OIR. Representative confocal immunofluorescence of retinal flatmounts stained for α-SMA (FIG. 11A) or NG2 (FIG. 11B), isolectin B4 (IB4), at P17 and P21 during normoxia and OIR. Scale bars represent 200 μm;

FIGS. 12A-C show that metformin inhibits senescence during OIR. FIG. 12A: Representative SA-β-gal staining of cryosections from P14 and P17 OIR retinas injected with metformin (10 μg/μl, at P12) or vehicle (PBS). FIG. 12B: Representative confocal immunofluorescence of TUNEL (left) and DAPI (middle) on cryosectionned P14 OIR retinas treated or not with metformin. The retinal ganglion cells (GCL), inner nuclear layer (INL) and outer nuclear layer (ONL) are shown for orientation. FIG. 12C: Western blot analysis of cleaved caspase-3 protein expression level during OIR (P14, P17 and P21). β-actin is used as a loading control. Scale bars are 200 μm;

FIGS. 13A-B show genes unaffected by Metformin and Aflibercept in OIR. FIG. 13A: RT-qPCR for levels of Vegf-a, Vegf-c, Vegfr-1 and Vegfr-2 measured in retinas of P14 OIR mice intravitreally injected with metformin or vehicle at P12 (β-actin was used as a reference gene); FIG. 13B: RT-qPCR for levels of Cdkn2a, Tp53, Il1β, Tgf-β1 and Sema3a measured in retinas of P14OIR mice intravitreally injected with Aflibercept or vehicle at P12 (β-actin was used as a reference gene);

FIG. 14 shows how the quantification of retinal SA-β-gal is performed. Schematic description of quantification of SA-β-gal staining on flatmount retinas (or sagittal eye sections) using Image J software analysis;

FIGS. 15A-E shows the structures of exemplary biguanide compounds and inhibitors of IRE1α. FIG. 15A: biguanide (CAS #56-03-1); FIG. 15B: Metformin (N,N-Dimethylimidodicarbonimidic diamide; CAS #657-24-9); FIG. 15C: Buformin (1-butylbiguanide, CAS #692-13-7); and (FIG. 15D) Phenformin (2-(N-phenethylcarbamimidoyl)guanidine, CAS #114-86-3); FIG. 15E: “Compound 3” IRE1α inhibitor which inhibits the RNAse activity of IRE1α (Wang et al., 2012, Nat. Chem. Bio. 8(12): 982-989);

FIG. 16 shows the amino acid sequence of human SEMA3A precursor protein. This sequence (SEQ ID NO: 50) is further processed into mature form by removal of the signal peptide (amino acid 1-21);

FIGS. 17A-B show an alignment between rat (Access. Nos. EDL96784, NP_659566, SEQ ID NO: 48), human (Accession No. NM003873, SEQ ID NO: 96) and mouse (Accession No. NP_032763, SEQ ID NO: 48) NRP1 protein sequences together with an NRP1 consensus sequence (SEQ ID NO: 47). The NRP1 signal domain (amino acids 1-20/1-21/1-27), subdomain a1 (from about aa22 to about aa148), subdomain a2 (from about aa149 to about aa275), subdomain b1 (from about aa276 to about aa428) and subdomain b2 (from about aa429 to about aa589), domain c (from about aa590 to about aa859), transmembrane domain (from about aa860 to about aa883) and cytoplasmic domain (from about aa884 to about aa923) are identified;

FIGS. 18A-G show an amino acid sequence alignment between exemplary traps of the present invention (see Tables 2 and 9 for the SEQ ID Nos corresponding to each trap shown);

FIGS. 19A-C show human soluble Neuropilin-1 (NRP1) protein sequences. FIG. 18A: NRP1 isoform b/s12 (644 amino acids; Ref seq: NP_001019799.1; NM_001024628.2; Uniprot: O-14786-2, SEQ ID NO: 44); FIG. 18B: NRP1 isoform c/Siv (609 amino acids; Ref seq: NP_001019800.1; NM001024629.2; Uniprot: O14786, SEQ ID NO: 45); FIG. 18C: NRP1 isoform SIII (704 amino acids; Ensembl: ENSP00000363956, SEQ ID NO: 46); and

FIGS. 20A-B show attenuation of cellular senescence by a single injection of an NRP1 trap. FIG. 20A: Representative P17 OIR flatmount retinas labeled with SA-β-gal in mice intravitreally injected with traps M and G at P12. FIG. 20B: Quantification of SA-β-gal staining reveals a significant attenuation of cellular senescence when mice receive a single injection of TrapM or TrapG.

FIGS. 21A-G show that NRP1 expressing macrophages accumulate in adipose tissue during diet-induced obesity. FIG. 21A: NRP1 expression level of eosinophils (adipose tissue, peripheral blood), neutrophils (blood, synovial fluid, bone marrow), monocytes (classical: MHCII+, MHCII−, MHCII− LN, bone marrow; non-classical: MHCII intermediate, MHCII high, MHCII−, bone marrow), and macrophages (adipose tissue, bone marrow, red pulp, lung resident, lung CD11b+, central nervous system, steady state peritoneal (high), steady state peritoneal (low), small intestine serosal, small intestine lamina propria) (n=1-4 per group). FIG. 21B: Adipose tissue macrophage (ATM) population FACS in 10 week high fat diet (HFD), and aged-matched control on regular diet (RD) C57BL/6 in white adipose tissue (WAT); FIG. 21C: NRP1 expression levels (n=9 per group) in macrophage (ATM). (FIGS. 21D-G) mRNA expression of NRP1 ligands: (FIG. 21D) Sema3a; (FIG. 21E) Vegfa; (FIG. 21F) Vegfb; and (FIG. 21G) Tgfb1 in RD and 10 week HFD C57BL/6 retroperitoneal white adipose tissue (RPWAT) (n=5 per group). Data are represented as mean±S.E.M. Student's unpaired t-test (B-G) *p<0.05, **p<0.01, ***p<0.001;

FIGS. 22A-K shows that NRP1 promotes FA uptake and phagocytosis. FIG. 22A: Acute BODIPY™ uptake within control and LysM-Cre-NRP1^{fl/fl} macrophages (n=7-8 per group). BODIPY™ uptake within (FIG. 22B) retroperitoneal white adipose tissue (RPWAT), (FIG. 22C) Liver, (FIG. 22D) Plasma and (FIG. 22E) Heart of HFD fed control and LysM-Cre-NRP1^{fl/fl} mice (n=6 per group). (FIG. 22F) ORO (Oil red O) stain of control and (FIG. 22F) LysM-Cre-NRP1^{fl/fl} macrophages incubated in adipocyte conditioned medium, FIG. 22H: Quantification of ORO stain of control and LysM-Cre-NRP1^{fl/fl} macrophages incubated in adipocyte conditioned medium (DMEM and insulin), (FIG. 22I) DMEM and insulin, (FIG. 22J) DMEM, (FIG. 22K) Macrophage medium (F12) (n=18-35 per group). Data are represented as mean±S.E.M. Student's unpaired t-test, *p<0.05, **p<0.01, ***p<0.001;

FIG. 23 shows that macrophages lacking NRP1 have a decreased phagocytic capacity. Phagocytosis was measured with the pHrodo green zymosan bioparticles conjugate in LysM-Cre-NRP1^{fl/fl} and control macrophages. pHrodo fluorescence was detected in control and LysM-Cre-NRP1^{fl/fl} macrophages (n=8 per group). Data represented as mean±S.E.M. Student's unpaired t-test, **p<0.01;

FIG. 24 shows that NRP1 polypeptide trap prevents weight gain in mice fed a high fat diet (see Example 4). The effect of an NRP1 trap on weight gain was assessed. An adeno virus expressing a soluble NRP1 trap comprising domains a1, a2 and b1 of NRP1 (Trap M, FIG. 9A); Adeno GFP; or saline (control) was administered to male mice and at the same time mice were switched from a regular diet to a high fat diet (HFD, T0). Weight gain was monitored over a period of 10 weeks. Data are presented as mean±SEM. Student's unpaired t-test, *p<0.05, **p<0.01, Saline vs Adeno Trap M, Two-way Anova, Bonferroni posttest, wherein N=5;

FIGS. 25A-B show Glucose tolerance test in mice expressing Trap M. FIG. 25A: Glycemia (mM) at different time-points of mice fed a HFD after an intraperitoneal injection of 2 g of glucose/kg mice. C57BI6/J mice at 6-8 weeks of age were intravenously injected with saline or Adeno-Trap M (0.25×10¹⁰ PFU/injection). Mice were fed a high fat diet right after injection. Glycemia was assessed at different time-points after the intraperitoneal injection of 2 g of glucose/kg mice. FIG. 25B: Area under the curves shown in (A) **P<0.01 (Adeno GFP vs Adeno Trap M) in Two-way Anova Bonferroni posttest, wherein N=5;

FIG. 26 shows analysis of pharmacodynamic properties of Trap M following systemic injection. C57BI6 mice (aged 6-8 weeks) were intravenously tail vein injected with purified Traps M (0.5 mg/kg dose), and serum samples were collected 6.24 and 48 hrs post injection. Traps M captured from approximatively 75 ul serum using IMAC sepharose was detected by immunoblotting with an anti-human NRP1 (cubAl domain);

FIG. 27 shows Cos cell expression of traps G, A, D and M following transduction with adenovirus stocks. Cos cells were transduced with the indicated adenovirus stocks. Traps G and M were purified from transduced cells supernatants using IMAC sepharose while Traps A and D were enriched using protein A/G sepharose. Traps were detected by immunoblotting with an anti-human NRP1 (cubAl domain). Legends: NI) supernatant from non-infected Cos cells, GFP supernatant from Adeno-Green fluorescent protein (GFP) infected Cos cells S1) Lipofectamine™ 2000 transfection adenovirus stock, S2) Effectene transfection adenovirus stock, S3) PEI transfection adenovirus stock, A2) Adenovirus stock amplification round 2, A3) Adenovirus stock amplification round 3. Where specifies purification were done with IMAC sepharose (IMAC) or Protein A/G sepharose (A/G);

FIG. 28 shows analysis of the pharmacological distribution of Trap M following systemic infection with the Adenovirus-Trap M construct. C57BI6 mice (aged 6-8 weeks) were intravenously tail vein injected with adenovirus-Trap M or control adenovirus-GFP stocks. Mice were sacrificed 2 weeks post infection and serum, kidneys and liver tissues were collected and stored at −80° C. until analysis. Trap M was captured from serum (approximatively 75 ul) or tissues lysates from kidney or liver (approximatively 40 mg) lysed in PBS/2% triton x-100 using IMAC sepharose and was detected by immunoblotting with an anti-human NRP1 (cubAl domain). Legend; 1) Non-infected mice, 2) Adeno-GFP infected mice and 3) Adeno-Trap M infected mice; and

FIG. 29 shows the expression of trap M two weeks post adenovirus infection. Trap M was captured from blood (approximatively 25 ul) lysed in PBS 2% triton X-100 using IMAC sepharose and was detected by immunoblotting with an anti-human NRP1 (cubAl domain). Legend; NI: Non-infected mice.

### DESCRIPTION OF ILLUSTRATIVE EMBODIMENTS

### 1. Role of Cellular Senescence and SASP in Ocular Vascular Diseases

The data presented herein establish a novel role for cellular senescence in weathering ischemia and modulating angiogenesis in ocular vascular diseases. Indeed a transient accumulation of senescent cells was established in different subcellular populations of the retina in different models of retinopathies. More particularly, it was found that by adopting a SASP, retinal neurons stimulate production of a series of paracrine factors and inflammatory cues that spread senescence to retinal microglia as well as endothelial cells and further exacerbate pathological pre-retinal angiogenesis. Applicants have shown that modulation of cellular senescence through the inhibition of the SASP (e.g., administration of biguanide compounds (e.g., metformin) or pharmacological or genetic inhibition of IRE1α) inhibits ischemia-induced senescence, increases vascular regeneration and suppresses pathological neovascularization in models of vascular ocular diseases. Therefore the SASP was shown to participate in mediating pathological vessel growth, with ischemic cells entering a state of premature senescence and secreting inflammatory cytokines that drive paracrine senescence, exacerbates destructive angiogenesis and hinders reparative vascular regeneration.

Data presented herein support that in the context of ocular vascular diseases such as retinopathies, cellular senescence exerts dichotomous roles within the same disease in that it first likely protects neurons from cell death yet concurrently prevents them from triggering programs of reparative angiogenesis. In addition, the paracrine senescence observed and associated production of vasomodulatory factors in retinopathies, contributes to repelling neovessels to the physiologically avascular vitreous and may promote premature aging-related complication in retinal vasculature. This is particularly relevant in light of the increased incidence of neovascular ocular disease associated with age such as age-related macular degeneration and diabetic retinopathy. Hence preventing cellular senescence during a phase of pathological neovascularization with administration of modulators of senescence could therefore represent a simple therapeutic solution for ocular vascular diseases and disorders such as retinal vasculopathies.

### 2. Methods of Treating or Preventing Vascular Eye Diseases Involving Cellular Senescence

Thus, according to an aspect of the present invention, compositions and methods are provided for treating and/or preventing at least one symptom or indication of a vascular eye disease or disorder in a subject. The methods according to this aspect of the invention comprise administering an inhibitor of the SASP (e.g., a biguanide compound such as metformin) to the subject. In certain aspects the inhibitor of the SASP is administered locally, in the eye of the subject (e.g., topically or intravitreally as opposed to, for example, systemically). Ocular administration is particularly preferred in the case of biguanide compounds such as metformin because systemic administration will generally not allow the compound to reach its target site because of the presence of the blood retinal barrier. In embodiments, the vascular eye disease or disorder is secondary to cellular ischemia.

Vascular eye diseases or conditions that may benefit from inhibition of the SASP in accordance with the present invention include any disease, disorder or condition characterized by abnormal angiogenesis (e.g., pathological neovascularization and/or reduced vascular regeneration). These diseases may be caused by a reduction (transient or sustained/chronic) of metabolic supply (e.g., oxygen, blood, nutrients) to cells which contribute to the normal eye function (e.g., ocular vascular cells, retinal cells, neurons, microglia) leading the presence of senescent cells (or cells harboring a senescence phenotype). Such condition may be present following an ischemic event but is not so limited. As used herein, the term “vascular eye disease or disorder” or “vascular eye disease or condition” thus refers to a disease, disorder or condition that affects the normal physiology of blood vessels in the eye. Non-limiting examples of such ocular eye diseases or conditions comprise: diabetic retinopathy, retinopathy of prematurity, ischemic retinopathy, diabetic macular edema, age-related macular degeneration, retinal neovascularisation, central retinal vein occlusion, branched retinal vein occlusion, choroidal neovascularization, polypoidal choroidal vasculopathy, physical injury to the eye, glaucoma, rhegmatogenous retinal detachment (RRD), retinal vasculitis, retinal macroaneurysm, retinal microaneurysm, Fuch's dystrophy, ischemic optic neuropathy, juvenile macular degeneration, macular telangiectasia, optic neuritis, usher syndrome, retinitis pigmentosa, uveitis, stangardt disease, Leber's congenital amaurosis (LCA). In embodiments, the vascular eye disease or disorder is an ischemic retinopathy. In embodiments, the ischemic retinopathy is associated with diabetic retinopathy, retinopathy or prematurity, ocular vein occlusion, central retinal vein occlusion or branched retinal vein occlusion.

Compounds or agents that inhibit the SASP in accordance with the present invention include biguanide compounds (e.g., metformin), mTor inhibitors (e.g., rapalogue, Torin 1) and/or inhibitors of IRE1α expression (e.g., antisense, shRNAs, etc.), IRE1α activation (S724 phosphorylation) and/or IRE1α RNAse activity (e.g., pharmacological inhibitors/antagonists). Generally, “IRE1α inhibitors” which inhibit the SASP in accordance with the present invention are those which ultimately reduce or abrogate IRE1α RNAse activity.

In particular aspects, compounds and agents that inhibit the SASP and prevent and/or attenuate cellular senescence in the context of vascular eye diseases and disorders (e.g., involving proliferative retinopathies) increase physiological angiogenesis (i.e., beneficial angiogenesis) and reduce pathological angiogenesis (pathological neovascularization) and thus promote tissue repair.

IRE1α is an enzyme that in humans is encoded by the ERN1 gene (Entrez: 2081, Ensembl ENSG00000178607, Uniprot: O75460, Refseq mRNA: NM_152461, NM_001433, Refseq (protein): NP_001424.3). This protein possesses intrinsic kinase activity and an endoribonuclease activity and it is important in altering gene expression as a response to endoplasmic reticulum-based stress signals (mainly the unfolded protein response (UPR)). Two alternatively spliced transcript variants encoding different isoforms have been found for this gene. IRE1α possesses two functional enzymatic domains, an endonuclease and a trans-autophosphorylation kinase domain. Upon activation, IRE1α oligomerizes and carries out an unconventional RNA splicing activity, removing an intron from the X-box binding protein 1 (XBP1) mRNA, and allowing it to become translated into a functional transcription factor, XBP1s. XBP1s upregulates ER chaperones and endoplasmic reticulum associated degradation (ERAD) genes that facilitate recovery from ER stress. Compounds which inhibit IRE1α (i.e., inhibitors) are also known in the art.

As used herein the term “IRE1α inhibitor” or “IRE1α antagonist” refers to an agent able to reduce or block IRE1α-mediated cell signaling associated with cellular senescence and the induction of the SASP (i.e., IRE1α ribonuclease activity and XBP1 processing). Non-limiting examples include an agent which reduces or blocks the expression (transcription or translation) of IRE1α, an agent able to reduce or block IRE1α activation (e.g., S724 phosphorylation and/or IRE1α dimerization). Without being so limited, the agent can be natural or synthetic and can be small molecule or a protein/polypeptide/nucleic acid such as but not limited to an antisense or a shRNA specific to an IRE1α nucleic acid sequence encoding an IRE1α protein or any pharmacological inhibitor described herein. IRE1α inhibitors or IRE1α antagonists of the present invention binds to IRE1α nucleic acid or IRE1α protein to reduce IRE1α expression, activation or activity and ultimately lead to a reduction of IRE1α RNAse activity within the cell.

Inhibitors targeting the catalytic core of the RNase domain and/or the ATP-binding pocket of the kinase domain have been described. Non-limiting examples of inhibitors targeting the RNAse binding pocket include salicylaldehydes (e.g., 3-methoxy-6-bromosalicylaldehyde-Volkmann et al., 2011, JBC 286(14): 12743-12755, PMCID: PMC3069474), 4μ8C, MKC-3946, STF-083010, and toyocamycin. Compounds that inhibit IRE1α's RNase activity through the kinase domain have also been identified and named “kinase inhibiting RNase attenuators” (KIRAs) and include KIRA3, and KIRA6 (Cas #1589527-65-0), which inhibit both the kinase and RNAse activities of IRE1α. Sunitinib and APY29 are examples of compounds which inhibit the ATP-binding pocket but allosterically activate the IRE1α RNase domain (Wang et al., 2012, Nat. Chem. Bio. 8(12): 982-989). Further kinase and/or RNAse inhibitors and activators of IRE1α are described in Wang Supra. In particular embodiment, IRE1α inhibitors which are used in accordance with the present invention inhibit the RNAse activity of IRE1α but not its kinase activity.

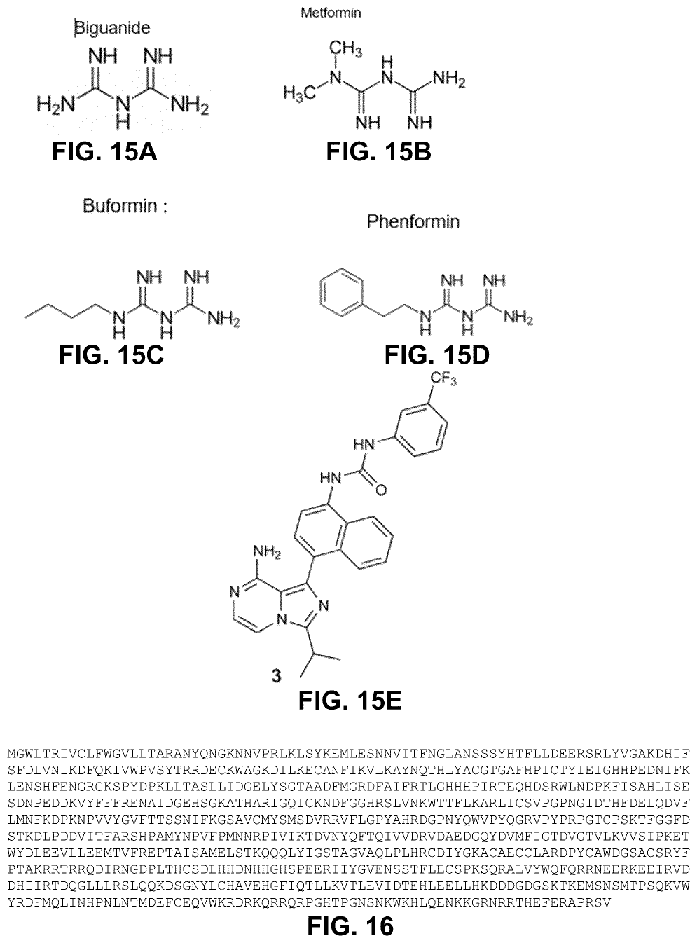

Biguanides are a class of organic compound with the formula HN(C(NH)NH₂)₂. These compounds were originally discovered in French Lilac (Galega officinalis) extracts and showed to lower blood glucose levels. They were thus originally used for the treatment of type 2 diabetes. A variety of derivatives of biguanide are used as pharmaceutical drugs for the treatment of diabetes but also for other diseases and conditions including polycystic ovary syndrome and cancer. Non-limiting examples include, metformin (N,N-Dimethylimidodicarbonimidic diamide (IUPAC name); CAS 657-24-9; DrugBank DB00331; ChemSpider 3949; Glucophage XR™; Carbophage SR™; Riomet™; Fortamet™; Glumetza™; Obimet™; gluformin™, Dianben™, Diabex™, Diaformin™, Siofor™, and Metfogamma™), buformin (1-butylbiguanide, CAS #692-13-7), Phenformin (2-(N-phenethylcarbamimidoyl)guanidine, CAS #114-86-3), Proguanil, (1-[amino-(4-chloroanilino)methylidene]-2-propan-2-ylguanidine, also known as chlorguanide), Chlorproguanil, Synthalin A, (1,1′-decane-1,10-diyldiguanidine, Cas #111-23-9) and Synthalin B, (1,1′-Dodecamethylenediguanidinium dichloride, Cas #61167-43-9). FIG. 15 shows the structure of biguanide and some functional derivatives that may be used in accordance with the present invention.

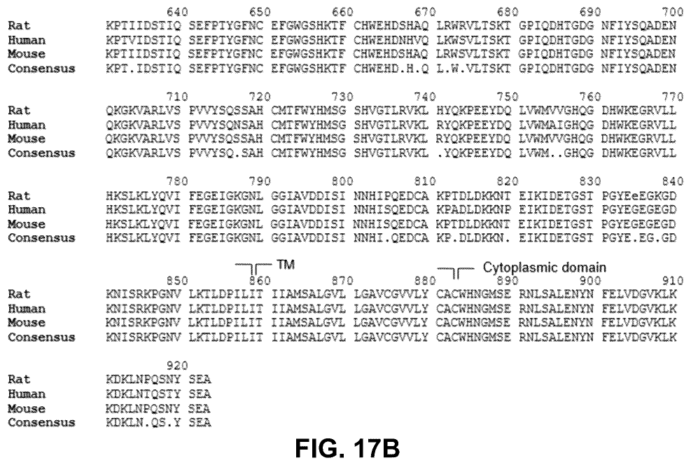

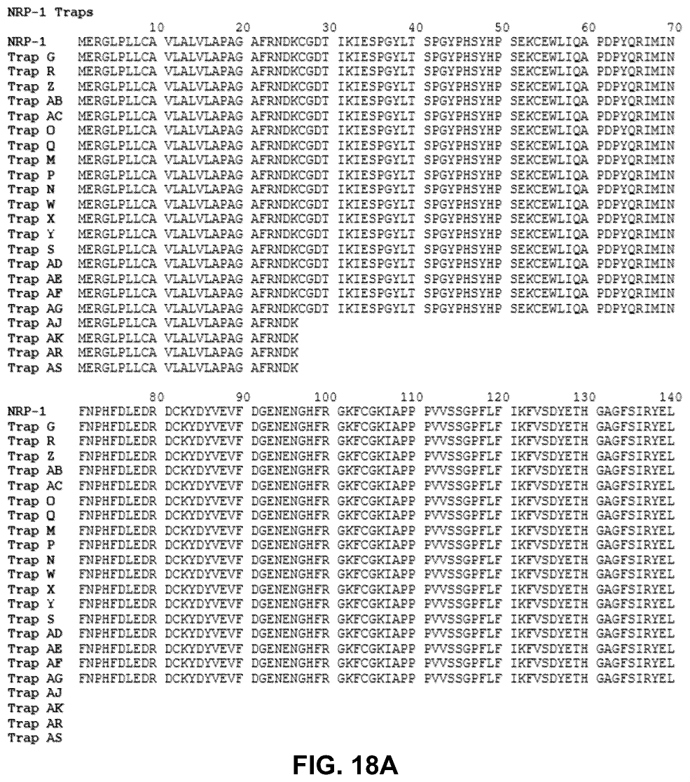

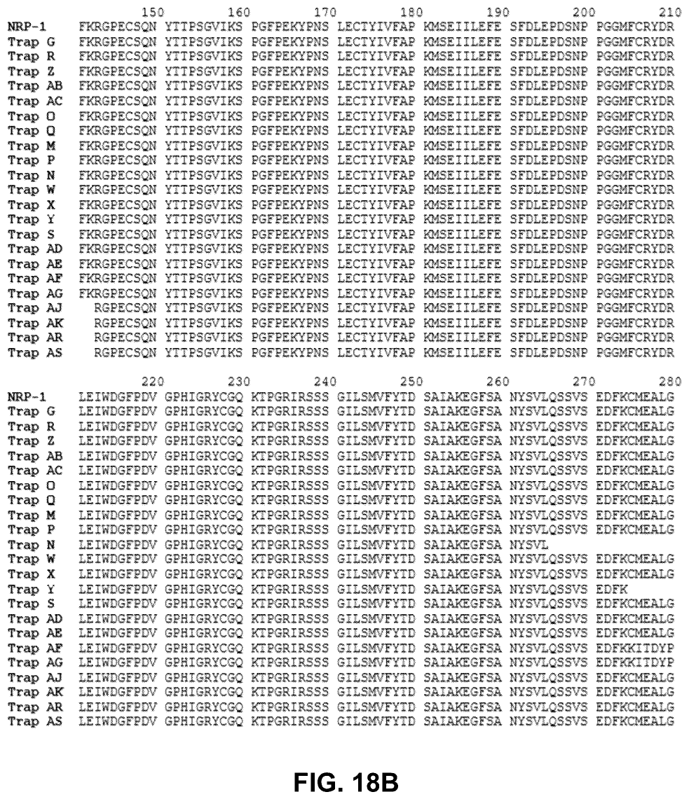

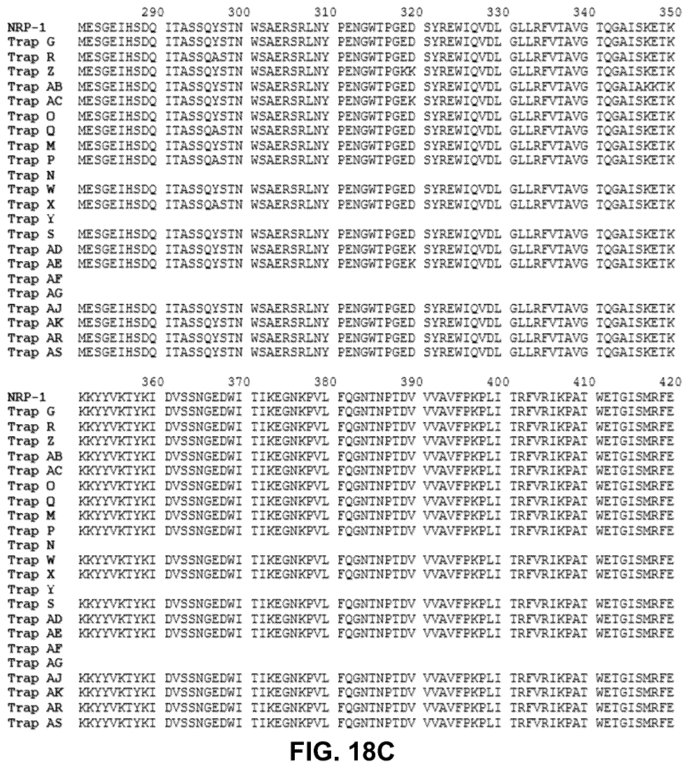

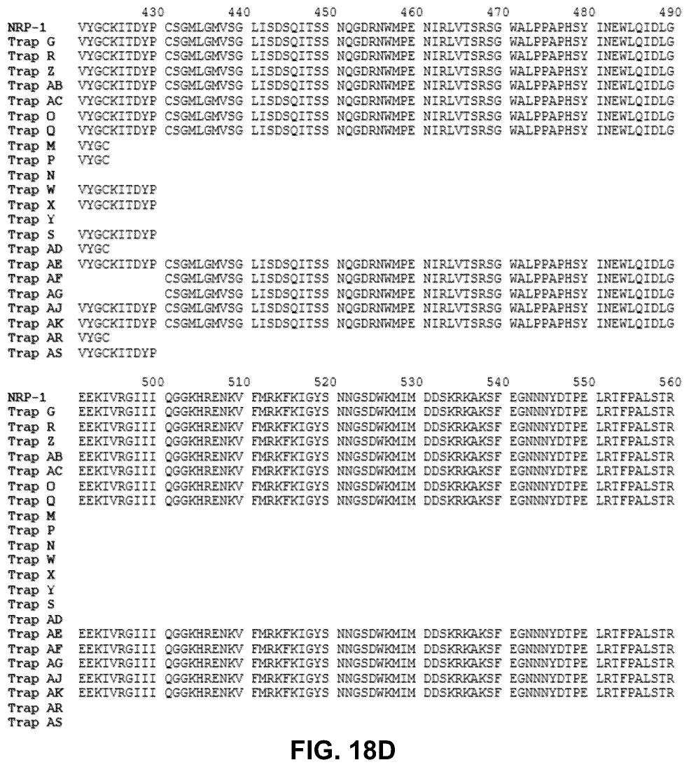

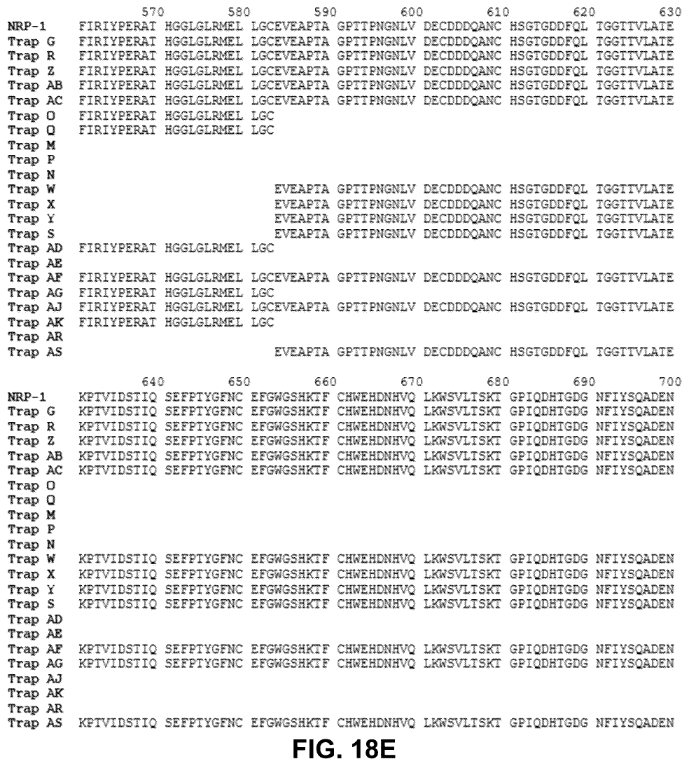

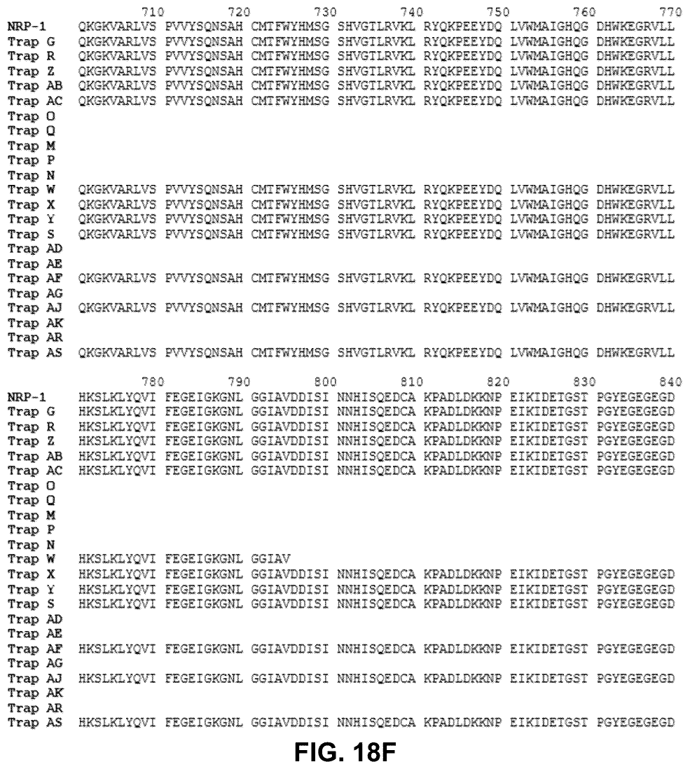

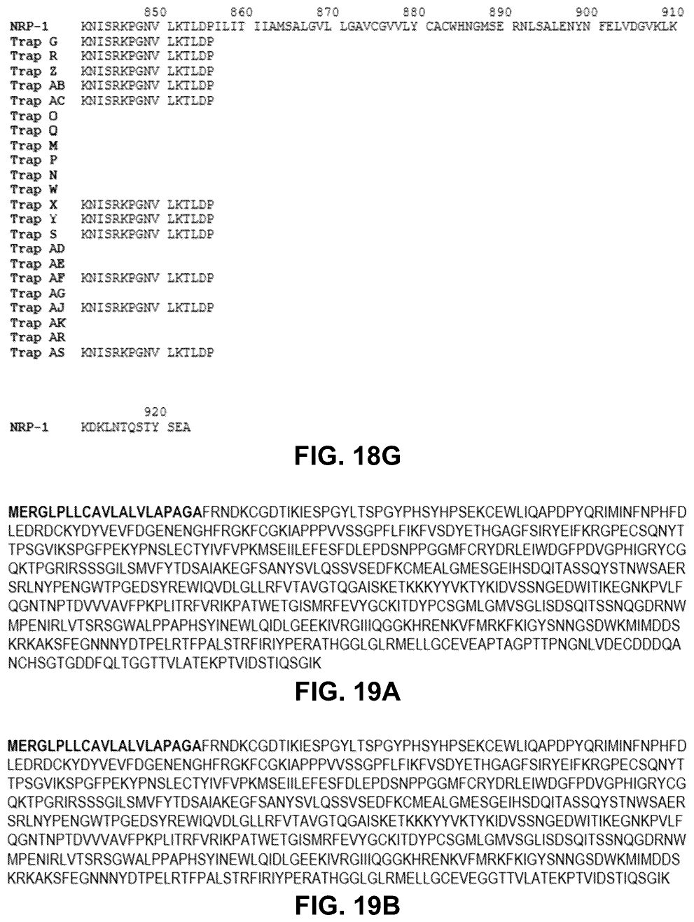

SASP inhibitors of the present invention may be administered in combination with other drugs used to treat vascular eye diseases and disorders including Angiopoietin-2 inhibitors (e.g., described in WO2016/085750), VEGF antagonists (e.g., anti VEGF antibodies (e.g., ranibizumab/LUCENTIS™)), small molecule VEGF inhibitors (e.g., sunetinib), VEGF-inhibiting fusion proteins (e.g., Aflibercept/EYELEA™) and/or SEMA3A antagonists (e.g., SEMA3a antibodies or NRP1 traps described below (see Table 2 and FIG. 18) or, for example, in WO 2016/033699).

### 3. Reduction or Prevention of Cellular Senescence and the SASP by Inhibiting IRE1α

Data presented herein further establish a role for IRE1α in modulating cellular senescence and the SASP. Cellular senescence, (including autocrine and/or paracrine) paracrine senescence can be inhibited or prevented by reducing IRE1α activity (i.e., IRE1α activation and cellular signalling).

IRE1α activity can be inhibited by a number of approaches. Inhibition of IRE1α cellular activity may be done directly by reducing IRE1α (i) nucleic acid or protein expression, (ii) activation (Serine 724 phosphorylation); and/or (iii) RNAse activity (and optionally, its kinase activity) in a cell. As noted above, IRE1α inhibitors are known in the art and include agents which inhibit IRE1α expression (e.g., IRE1α antisense of sh_RNAs), IRE1α activation (e.g., KIRA3, KIRA6) and/or IRE1α ribonuclease (and optionally kinase) activity (e.g., salicylaldehydes, 4μ8C, MKC-3946, STF-083010, KIRA3, KIRA6 and toyocamycin).

The present invention thus provides a method of inhibiting or preventing cellular senescence of a cell or induction of the senescence-associated secretory phenotype (SASP) in a cell comprising reducing IRE1α level or activity.

The present invention also concerns a method of inhibiting or preventing cellular senescence of a cell or induction of the senescence-associated secretory phenotype (SASP) in a cell comprising contacting the cell with an inhibitor of IRE1α.

Also provided is a method of inhibiting or preventing cellular senescence of a cell or induction of the senescence-associated secretory phenotype in a cell of a subject comprising administering to the subject an inhibitor of IRE1α.

The above methods may be useful in treating or preventing diseases or conditions in which cellular senescence is detrimental such as various age-related conditions (e.g., sarcopenia, neurodegeneration, thinning of the epidermis, skin wrinkling, hair loss and greying hair, cataract, obesity, metabolic syndrome, and other diseases of old age), chronic obstructive pulmonary disease (COPD), Idiopathic pulmonary fibrosis (IPF), atherosclerosis, osteoarthritis, osteoporosis, glaucoma, Parkinson's disease, intestinal bowel disease, intervertebral disc degeneration, brain aneurysm, aortic aneurysm, pancreatic fibrosis, vascular ocular diseases (e.g., retinal vascular diseases (proliferative retinopathies, diabetic retinopathy, ischemic retinopathies, macular degeneration, glaucoma) and cystic fibrosis. Inhibition or prevention of cellular senescence may also be useful during and/or after cancer treatment to alleviate side effects of chemotherapy/radiotherapy which include for example, metabolic dysfunction, accelerated aging, increased risk of cancer later in life. In embodiments, the senescence-associated diseases or conditions which are encompassed by the present invention exclude one or more vascular ocular diseases (e.g., retinal vascular diseases (proliferative retinopathies, diabetic retinopathy, ischemic retinopathies, macular degeneration, glaucoma)).

Various approaches are available for decreasing IRE1α expression and thus IRE1α-mediated cellular senescence. Non-limiting example includes the use of small hairpin shRNA (RNAi), antisense, ribozymes, TAL effectors targeting the IRE1α promoter or the like. Expression of shRNAs or similar inhibitory RNAs in cells can be obtained by delivery of plasmids or through viral (e.g., lentiviral vector, adenoviral vector, etc.) or bacterial vectors.

Therefore, in alternative embodiments, the invention provides antisense, shRNA molecules and ribozymes for exogenous administration to effect the degradation and/or inhibition of the translation of mRNA of interest. The present invention also provides vectors and host cells for delivering and/or expressing the antisense, shRNA molecules, ribozymes, etc. disclosed herein. The antisense, shRNA molecules and ribozymes preferably target mammalian (preferably human) IRE1α. Examples of therapeutic antisense oligonucleotide applications include: U.S. Pat. No. 5,135,917, issued Aug. 4, 1992; U.S. Pat. No. 5,098,890, issued Mar. 24, 1992; U.S. Pat. No. 5,087,617, issued Feb. 11, 1992; U.S. Pat. No. 5,166, 195 issued Nov. 24, 1992; U.S. Pat. No. 5,004,810, issued Apr. 2, 1991; U.S. Pat. No. 5, 194,428, issued Mar. 16, 1993; U.S. Pat. No. 4,806,463, issued Feb. 21, 1989; U.S. Pat. No. 5,286,717 issued Feb. 15, 1994; U.S. Pat. No. 5,276,019 and U.S. Pat. No. 5,264,423; BioWorld Today, Apr. 29, 1994, p. 3.

Preferably, in antisense molecules, there is a sufficient degree of complementarity to the mRNA of interest to avoid non-specific binding of the antisense molecule to non-target sequences under conditions in which specific binding is desired, such as under physiological conditions in the case of in vivo assays or therapeutic treatment or, in the case of in vitro assays, under conditions in which the assays are conducted. The target mRNA for antisense binding may include not only the information to encode a protein, but also associated ribonucleotides, which for example form the 5′-untranslated region, the 3′-untranslated region, the 5′ cap region and intron/exon junction ribonucleotides. A method of screening for antisense and ribozyme nucleic acids that may be used to provide such molecules as IRE1α inhibitors of the invention is disclosed in U.S. Pat. No. 5,932,435.

In some embodiments, the antisense oligonucleotides in accordance with this invention may comprise from about 5 to about 100 nucleotide units. As will be appreciated, a nucleotide unit is a base-sugar combination (or a combination of analogous structures) suitably bound to an adjacent nucleotide unit through phosphodiester or other bonds forming a backbone structure.

In a further embodiment, expression of a nucleic acid encoding a polypeptide of interest (IRE1α), or a fragment thereof, may be inhibited or prevented using RNA interference (RNAi) technology, a type of post-transcriptional gene silencing. RNAi may be used to create a pseudo “knockout”, i.e. a system in which the expression of the product encoded by a gene or coding region of interest is reduced, resulting in an overall reduction of the activity of the encoded product in a system. As such, RNAi may be performed to target a nucleic acid of interest or fragment or variant thereof, to in turn reduce its expression and the level of activity of the product which it encodes. Such a system may be used for functional studies of the product, as well as to treat disorders related to the activity of such a product. RNAi is described in for example published U.S. patent applications Ser. No. 20/020,173478 (Gewirtz; published Nov. 21, 2002) and 20020132788 (Lewis et al.; published Nov. 7, 2002). Reagents and kits for performing RNAi are available commercially from for example Ambion Inc. (Austin, TX, USA) and New England Biolabs Inc. (Beverly, MA, USA).

The initial agent for RNAi in some systems is a dsRNA molecule corresponding to a target nucleic acid. The dsRNA (e.g., shRNA) is then thought to be cleaved into short interfering RNAs (siRNAs) which are 21-23 nucleotides in length (19-21 bp duplexes, each with 2 nucleotide 3′ overhangs). The enzyme thought to effect this first cleavage step has been referred to as “Dicer” and is categorized as a member of the RNase III family of dsRNA-specific ribonucleases. Alternatively, RNAi may be effected via directly introducing into the cell, or generating within the cell by introducing into the cell a suitable precursor (e.g. vector (viral vector such as an adenoviral vector) encoding precursor(s), etc.) of such an siRNA or siRNA-like molecule. An siRNA may then associate with other intracellular components to form an RNA-induced silencing complex (RISC). The RISC thus formed may subsequently target a transcript of interest via base-pairing interactions between its siRNA component and the target transcript by virtue of homology, resulting in the cleavage of the target transcript approximately 12 nucleotides from the 3′ end of the siRNA. Thus the target mRNA is cleaved and the level of protein product it encodes is reduced.

RNAi may be effected by the introduction of suitable in vitro synthesized siRNA (shRNAs) or siRNA-like molecules into cells. RNAi may for example be performed using chemically-synthesized RNA. Alternatively, suitable expression vectors may be used to transcribe such RNA either in vitro or in vivo. In vitro transcription of sense and antisense strands (encoded by sequences present on the same vector or on separate vectors) may be effected using for example T7 RNA polymerase, in which case the vector may comprise a suitable coding sequence operably-linked to a T7 promoter. The in vitro-transcribed RNA may in embodiments be processed (e.g. using E. coli RNase III) in vitro to a size conducive to RNAi. The sense and antisense transcripts are combined to form an RNA duplex which is introduced into a target cell of interest. Other vectors may be used, which express small hairpin RNAs (shRNAs) which can be processed into siRNA-like molecules. Various vector-based methods and various methods for introducing such vectors into cells, either in vitro or in vivo (e.g. gene therapy) are known in the art.

Accordingly, in an embodiment expression of a nucleic acid encoding a polypeptide of interest (IRE1α), or a fragment thereof, may be inhibited by introducing into or generating within a cell an siRNA or siRNA-like molecule corresponding to a nucleic acid encoding a polypeptide of interest (e.g. IRE1α), or a fragment thereof, or to an nucleic acid homologous thereto. “siRNA-like molecule” refers to a nucleic acid molecule similar to an siRNA (e.g. in size and structure) and capable of eliciting siRNA activity, i.e. to effect the RNAi-mediated inhibition of expression. In various embodiments such a method may entail the direct administration of the siRNA or siRNA-like molecule into a cell, or use of the vector-based methods described above. In an embodiment, the siRNA or siRNA-like molecule is less than about 30 nucleotides in length. In a further embodiment, the siRNA or siRNA-like molecule is about 21-23 nucleotides in length. In an embodiment, siRNA or siRNA-like molecule comprises a 19-21 bp duplex portion, each strand having a 2 nucleotide 3′ overhang. In embodiments, the siRNA or siRNA-like molecule is substantially identical to a nucleic acid encoding a polypeptide of interest, or a fragment or variant (or a fragment of a variant) thereof. Such a variant is capable of encoding a protein having activity similar to the polypeptide of interest.

### 4. Methods of Promoting Cellular Senescence by Increasing IRE1α Ribonuclease Activity

Under certain conditions, stimulation of cellular senescence may be beneficial. Cellular senescence, including autocrine and paracrine senescence can be promoted or induced by stimulating or increasing IRE1α activity (i.e., IRE1α RNAse activity and cellular signaling). IRE1α activity can be increased by a number of approaches including by increasing the expression of IRE1α in a cell or by contacting a cell with a compound which activates IRE1α RNAse activity (e.g., APY29, Sunitinib or compound 3 described in Joshi et al., 2015, 6(15): 1309-1335).

Methods of promoting cellular senescence may be useful in diseases and conditions where senescence has beneficial effects such as tissue repair, wound healing, liver fibrosis, renal fibrosis, myocardial infarction cardiac fibrosis, atherosclerosis, pulmonary hypertension and cancer.

### 5. Compositions and Methods for Modulating Cellular Senescence Comprising SEMA3A Modulators

The Class 3 Semaphorins (Sema3s) are a sub-family of proteins whose known biological roles are varied and growing. The mechanism of action of the Sema3s requires binding to transmembrane receptors that comprise heteromeric complexes of Neuropilins, Plexins and cell adhesion molecules (CAMs). The SEMA3A gene (GeneCard ID: GC07M083955; Entrez Gene ID: 10371; Ensembl: ENSG00000075213) encodes a 771 amino acid protein (NP_006071.1; UniprotKB: Q14563, SEQ ID NO: 50) comprising a signal peptide, an Ig-like C2-type (immunoglobulin-like) domain, a PSI domain and a Sema domain (which is required for signaling). This secreted protein was first described as an axonal guidance cue but it has now been implicated in various physiological and pathological process including organ development, bone metabolism, angiogenesis, vascular permeability, growth cone collapse, myogenic regeneration and formation of neuromuscular junction, regulation of the immune system, inflammation, schizophrenia and retinal diseases such as diabetic retinopathy.

Sema3a generally signals through receptor complexes comprising Neuropilin-1 (NRP1) and a coreceptor (e.g., Class A plexins (e.g., PLXna1-Plxna4, Plxnd1), L1cam, chL1, Robo1). NRP1 (Ensemble; ENSG00000099250; ENST00000265371; Uniprot: 014786; OMIM: 602069; HGNC:8004; GeneCard ID: GC10M033216, SEQ ID NOs: 44-47, 95 and 96) is a single-pass transmembrane receptor with a large intracellular domain. The basic structure of neuropilin-1 comprises 5 domains: Three extracellular domains (a1a2 (CUB), b1b2 (FV/FVIII) and c (MAM)), a transmembrane domain and a cytoplasmic domain. The a1a2 domain is homologous to complement components C1r and C1s (CUB) which generally contain 4 cysteine residues forming disulfide bridges. This domain binds SEMA3A. Domains b1b2 (FV/FVIII) binds to VEGF. Amino acid Y297 in subdomain b1 is important for binding to VEGF as substitution of Y297 to an alanine significantly reduces VEGF binding to NRP1. Subdomain b1 also contributes to SEMA3A ligand binding. Indeed, Applicants have surprisingly found that substitution of Y297 (Y297A) also significantly reduce SEMA3A binding to NRP1. Crystallographic evidence revealed that VEGF165 and Sema3A do not directly compete for NRP1 but rather can simultaneously bind to NRP1 at distinct, non-overlapping sites.

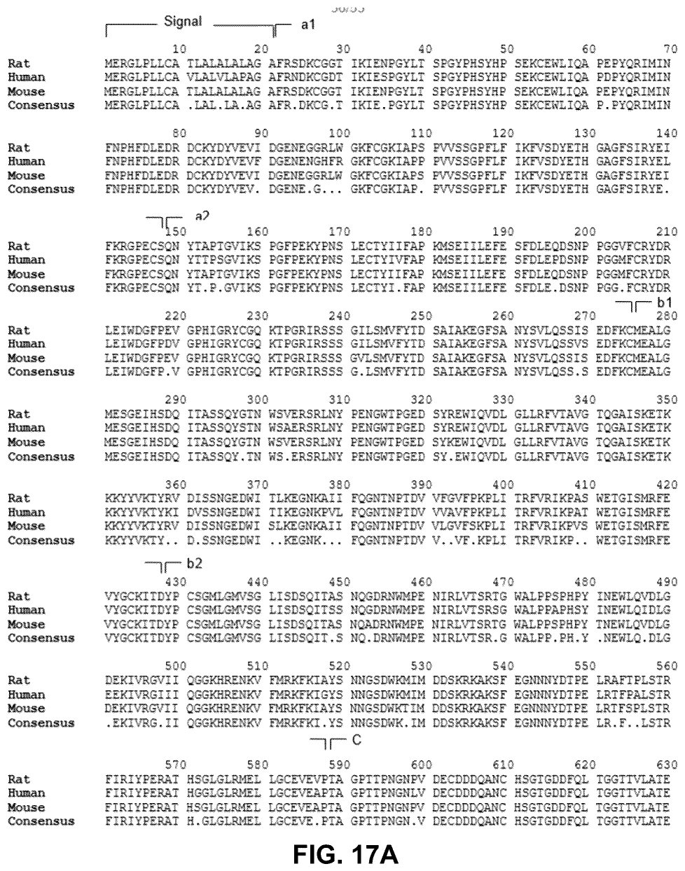

In addition to the transmembrane form (isoform 1, 923 aa, FIG. 17, SEQ ID NOs: 47, 95 and 96, NM003873; Uniprot: 014786-1), naturally occurring soluble NRP1 proteins (sNRP1) containing only part of the extracellular domain may be secreted by the cells. Different soluble forms have been described ranging from 551 to 704 amino acids (isoforms b/s12 NRP1 (644 aa; RefSeq: NP_001019799.1; NM_001024628.2, SEQ ID NO: 44), s11 NRP1 (704 aa; ENSP00000363956, SEQ ID NO: 46), sIII NRP1 (551 aa) and c/sIV NRP1 (609 aa; RefSeq: NP_001019800.1; NM001024629.2, SEQ ID NO: 45) (Cackowski et al., 2004, Genomics, 84(1): 82-94; Rossignol M et al., Genomics 2000; 70(2):211-222; and Gagnon ML et al., 2000, Proc. Natl. Acad. Sci. USA; 97(6):2573-2578)). The full-length form of the protein contains all 17 exons, whereas soluble isoforms are created by alternative splicing of the NRP1 gene or reading through introns. The b/s12, and c/sIV NRP1 isoforms contains the a1a2 and b1b2 domains and most of the b/c linker but no c-domain. Isoform sill contains the a1a2 domain, the b1 subdomain but only part of the b2 subdomain. The s11 NRP1 isoform contains the a1a2 and b1b2 domains, followed by the portion of the b/c linker encoded by exon 11 and 83 novel amino acids.

In a second aspect of the present invention, following studies in models of ischemic retinopathies, SEMA3A was surprisingly identified as a modulator of cellular senescence. Indeed an unsuspected mechanism triggered by neurons in devascularized retinal zones was identified where they enter a state of premature cellular senescence and adopt a senescence-associated secretory phenotype (SASP). Data described herein show that secretion of SEMA3A by senescent cells drives paracrine senescence through IRE1a and propagate senescence across the ischemic tissue to various cell types including neurons, microglia and the overlying vasculature (paracrine senescence). Furthermore, sustained exposure to SEMA3A was shown to activate IRE1a, induce senescence and drive the expression of a panel of genes known to be critical for promoting and reinforcing the senescent state such as Pai1, Il6, Il1β, TGF-β and Tp53. SEMA3A was also shown to promote senescence-associated DNA-damage foci expressing γH2AX that are hallmarks of cellular senescence. Notably, and as demonstrated herein, genetic interference against SEMA3A limits senescence and stimulates tissue repair.

The inventors have found that modulating SEMA3A levels or activity enables to control cellular senescence, and the secretion of proteins (typically pro-inflammatory cytokines of the SASP) that are released during cellular senescence. The inventors have found that by inhibiting SEMA3A expression or activity, cellular senescence can be prevented, limited or decreased and induction of SASP can be prevented or reduced. Similarly, increasing SEMA3A activity (e.g., by increasing its expression or by contacting cells with a SEMA3A polypeptide) promotes senescence and induces the SASP.

These data provide evidence for a previously unsuspected role for SEMA3A in modulating autocrine and paracrine senescence through the SASP in pathological processes and uncover the therapeutic benefits of modulating SEMA3A activity in diseases and conditions associated with senescence.

### (i) Methods of Inhibiting or Preventing Cellular Senescence by Inhibiting SEMA3A Signalling

Cellular senescence, including autocrine and paracrine senescence can be inhibited or prevented by reducing SEMA3A activity (i.e., SEMA3A cellular signalling). SEMA3A activity can be inhibited by a number of approaches. Inhibition of SEMA3A cellular activity may be done directly by (i) reducing SEMA3A nucleic acid or protein expression, (ii) by inhibiting its secretion by the cell; or (iii) by sequestering secreted SEMA3A in order to inhibit it's binding to its receptor on the cell surface; thereby preventing intracellular signalling, activation of IRE1α and initiation and/or propagation of cellular senescence. Non-limiting examples of agents and approaches for inhibiting SEMA3A activity include (i) antibodies against SEMA3A; (ii) antibodies against one of its receptor (i.e., competing with SEMA3A binding to its receptor); (iii) antisense and RNAi methods for reducing SEMA3A expression; and/or (iv) use of a soluble receptor or fragment thereof, acting as a functional SEMA3A trap.

The present invention thus provides a method of inhibiting or preventing cellular senescence of a cell or induction of the senescence-associated secretory phenotype (SASP) in a cell comprising reducing SEMA3A level or activity.

The present invention also concerns a method of inhibiting or preventing cellular senescence of a cell or induction of the senescence-associated secretory phenotype (SASP) in a cell comprising contacting said cell with a SEMA3A antagonist.

Also provided is a method of inhibiting or preventing cellular senescence or induction of the senescence-associated secretory phenotype in cells of a subject comprising administering to said subject an effective amount of a SEMA3A antagonist.

As used herein the term “SEMA3A inhibitor” or “SEMA3A antagonist” refers to an agent able to reduce or block SEMA3A-mediated cell signaling associated with SEMA3A induction of the SASP and SEMA3A induced cellular senescence. The “SEMA3A inhibitor” or “SEMA3A antagonist” of the present invention binds to or interacts with the SEMA3A polypeptide or SEMA3A nucleic acid (SEMA3A gene or mRNA) in order to reduce SEMA3A polypeptide expression or interaction with its cognate receptor) such that SEMA3A-mediated cell signaling is reduced or abrogated. Non-limiting examples include an agent which reduces or blocks the expression (transcription or translation) of SEMA3A, an agent able to reduce or block SEMA3A secretion or an agent able to reduce or block SEMA3A binding to its receptor NRP1. Without being so limited, the agent can be natural or synthetic and can be a protein/polypeptide, such as but not limited to, an antibody that specifically binds to SEMA3A or NRP1 receptor; a soluble NRP1 polypeptide or fragment thereof (e.g., an NRP1 trap which binds to SEMA3A), a peptide, a small molecule, a polynucleotide such as but not limited to an antisense or a shRNA specific to SEMA3A nucleic acid sequence encoding a SEMA3A protein or functional variant or fragment thereof.

The above methods may be useful in treating or preventing diseases or conditions in which cellular senescence is detrimental such as various age-related conditions (e.g., sarcopenia, neurodegeneration, thinning of the epidermis, skin wrinkling, hair loss and greying hair, cataract and other diseases of old age), chronic obstructive pulmonary disease (COPD), idiopathic pulmonary fibrosis (IPF), atherosclerosis, osteoarthritis, osteoporosis and Parkinson's disease, glaucoma, intestinal bowel disease, intervertebral disc degeneration, brain aneurysm, aortic aneurysm, pancreatic fibrosis and cystic fibrosis. Inhibition or prevention of cellular senescence may also be useful during and/or after cancer treatment to alleviate side effects of chemotherapy/radiotherapy which include for example, metabolic dysfunction, accelerated aging, increased risk of cancer later in life. In embodiments, the senescence-associated diseases or conditions which are encompassed by the present invention exclude ocular diseases (e.g., retinal vascular diseases (ischemic retinopathies, macular edema)), inflammation, cerebral ischemia, stroke or cancer.

a. Antibodies.

In a particular aspect of the present invention, SEMA3A activity (e.g., SEMA3A-induced IRE1α activation) can be inhibited by using SEMA3A antibodies. These antibodies bind to SEMA3A in such a way that it inhibits its binding to its cognate receptor, NRP1, thereby preventing SEMA3A-mediated cellular signaling (79, 80).

Alternatively, antibodies directly targeting the NRP1 receptor, which block the binding of SEMA3A to NRP1 may also be used. In a particular aspect of the present invention, antibodies targeting NRP1 block SEMA3A binding to the receptor but do not substantially interfere with VEGF binding to NRP1. In an embodiment, the NRP1 antibody binds to the a1a2 (A) domain of the NRP1 polypeptide.

As used herein, the term “SEMA3A antibody” refers to an antibody that specifically binds to (interacts with) a SEMA3A protein and displays no substantial binding to other naturally occurring proteins other than the ones sharing the same antigenic determinants as the SEMA3A protein. Similarly, the term “NRP1 antibody” refers to an antibody that specifically binds to (interacts with) a NRP1 protein and displays no substantial binding to other naturally occurring proteins other than the ones sharing the same antigenic determinants as the NRP1 protein. SEMA3A/NRP1 antibodies include polyclonal, monoclonal, humanized as well as chimeric antibodies. The term antibody or immunoglobulin is used in the broadest sense, and covers monoclonal antibodies (including full length monoclonal antibodies), polyclonal antibodies, multispecific antibodies and antibody fragments so long as they exhibit the desired biological activity. Antibody fragments comprise a portion of a full length antibody, generally an antigen binding or variable region thereof. Examples of antibody fragments include Fab, Fab′, F(ab′)2, and Fv fragments, diabodies, linear antibodies, single-chain antibody molecules, single domain antibodies (e.g., from camelids), shark NAR single domain antibodies, and multispecific antibodies formed from antibody fragments. Antibody fragments can also refer to binding moieties comprising CDRs or antigen binding domains including, but not limited to, VH regions (VH, VH-VH), anticalins, PepBodies™, antibody-T-cell epitope fusions (Troybodies) or Peptibodies.

Anti-human SEMA3A/NRP1 antibodies have been previously prepared (80) and are also commercially available from various sources including Santa Cruz. In general, techniques for preparing antibodies (including monoclonal antibodies and hybridomas) and for detecting antigens using antibodies are well known in the art and various protocols are well known and readily available.

b. Soluble Receptor or Fragment Thereof

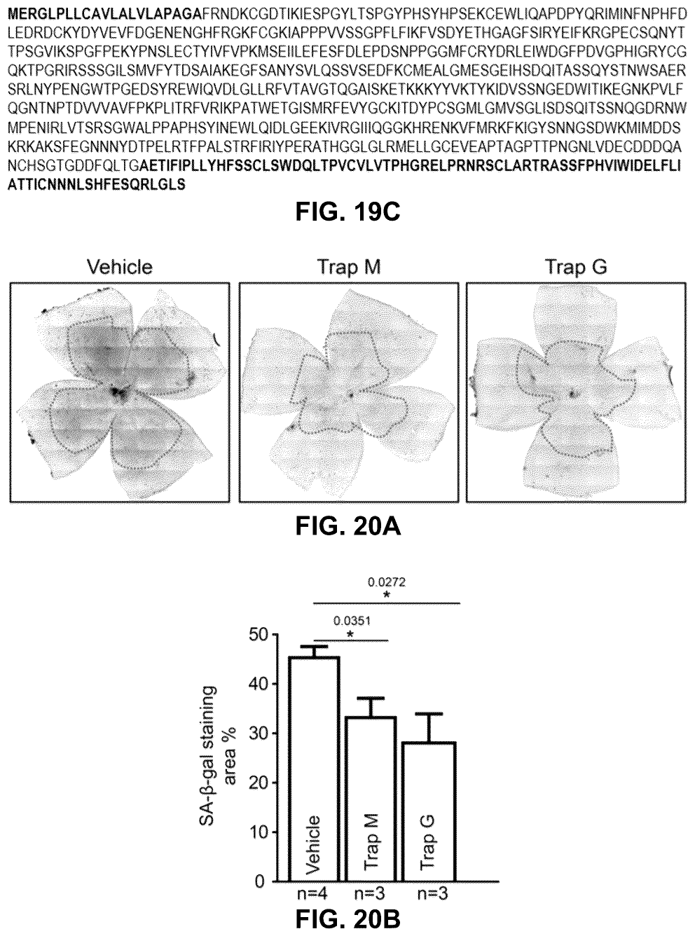

Modulation of Sema3-mediated cellular senescence can be achieved by using naturally occurring soluble NRP1 polypeptides or synthetic NRP1 polypeptides (e.g., produced in vitro in cell lines (recombinantly) or chemically synthesized). As used herein, the terms, “NRP1 trap”, or “NRP1 polypeptide trap” encompass a naturally occurring soluble NRP1 polypeptide (e.g., such as NRP1 secreted isoforms shown in FIGS. 17, 19, in Table 2 and in SEQ ID NOs: 44-47, 95 and 96), non-naturally occurring soluble NRP1 polypeptide and synthetic soluble NRP1 polypeptide including any functional soluble fragment of NRP1 or any functional variant of NRP1 which competes with endogenous (cellular, membrane-bound) NRP1 for SEMA3A ligand binding. In an embodiment, the NRP1 traps of the present invention do not exist in nature (i.e., are not naturally occurring) but are “derived” from naturally occurring NRP1 polypeptides (i.e. they are synthetic; e.g., NRP1 traps comprising the extracellular domain of NRP1 isoform 1 (e.g., aa 1-857 of transmembrane (cellular) NRP1) or a fragment or variant thereof). Generally, NRP1 traps of the present invention initially comprise a signal peptide at their N-terminal end (e.g., about amino acids 1-21 of the NRP1 amino acid sequence shown in FIG. 17) which is cleaved upon maturation and secretion by the cells. Accordingly, NRP1 polypeptide traps of the present invention lack amino acids 1-21 when administered as purified polypeptides or when prepared as pharmaceutical compositions comprising a purified or substantially pure form. Similarly, nucleic acids encoding NRP1 traps of the present invention comprise a polynucleotide sequence in 5′ which encodes for a signal peptide (e.g., first 63 nucleotides encoding for the first 21 amino acids at the N-terminal end) which will allow the NRP1 trap to be synthesized and secreted by the cells. Depending on the conditions and cell type, the length of the signal peptide removed for secretion may vary. Removal of aa 1-20, 1-21 and 1-27 (see FIGS. 17-18 and for example, SEQ ID NOs: 47, 95 and 96) have been described. In a particular embodiment, the signal peptide corresponds to the first 21 amino acids of the NRP1 polypeptide set forth in FIG. 17 or SEQ ID NO: 47, 95 or 96.

TABLE 1
NRP1 protein domains

| | Amino acid (with reference to |
|---|---|
| Domain | SEQ ID NOs 47, 95 and 96) |
| Signal Peptide (SP) | 1-20, 1-21 or 1-27 depending on condition |
| | and/or cell type |
| a1 (CUB 1) | From end of signal peptide to about aa 141 |
| a2 (CUB 2) | From about aa 147 to about aa 265 |
| b1 (F5/8 Cl) | From about aa 275 to about aa 424 |
| b2 (F5/8 C2) | From about aa 431 to about aa 583 |
| c (MAM) | From about aa 591 to about aa 859 |
| Transmembrane | From about aa 860 to about 882 |
| Cytoplasmic | From about aa 883 to aa 923 |

Non-limiting examples of NRP1 traps that may be used in accordance with the present invention include naturally occurring soluble NRP1 set forth in SEQ ID NOs; 44-47, 95 and 96, NRP1 traps described in Table 2 below, in FIG. 18, and in WO 2016/03699, which is incorporated herein by reference. Of course, the mature, active form of soluble NRP1 traps which are administered to a subject or contacted with a cell lack the portion of the aa acid sequence corresponding to the signal peptide as well as the transmembrane and cytoplasmic domain normally present in cellular, membrane-bound NRP1.

TABLE 2
Exemplary NRP1 secreted isoforms and exemplary NRP1 traps of the present invention tested for Sema3A binding:

| | | Amino | | | Deleted aa** | | SEQ |
|---|---|---|---|---|---|---|---|
| | | acids | Amino acids | | with reference to | Binding | ID |
| | | including | in mature trap | | full length NRP1 | to | Nos: |
| Trap | Description | SP | without SP* | Mutation(s) | (FIG. 17) | SEMA3A | nts/aa |
| Iso-b | a1a2-b1b2-c (in part) | 1-644 | About 22-644; | | Δ645-923 | Yes | 44 |
| | Secreted NRP1 isoform-b/S₁₂; | E642G; | E642G; F6431; | | | | |
| | 644 amino acids | F643I; | P644K | | | | |
| | Uniprot O14786-2; | P644K; | | | | | |
| | Refseq: NP_001019799.1; | | | | | | |
| | NM_001024628.2 | | | | | | |
| Iso-c | a1a2-b1b2-c (in part) | 1-586 and | About 22-586 | | Δ587-621 | Yes | 45 |
| | Secreted NRP1 isoform-c/Sᵢᵥ; | 622-644; | and 622-644; | | | | |
| | 609 amino acids | E642G; | E642G; | | | | |
| | Uniprot: O14786 | F643I; | F643I; P644K | | | | |
| | RefSeq: NP_001019800.1; | P644K; | | | | | |
| | NM001024629.2 | | | | | | |
| Iso-d | a1a2-b1b2-c (in part) | 1-622 and | About 22-704 | | Δ623-923 | Yes | 46 |
| | Secreted NRP1 isoform-b/S₁₁; | a novel 83 | | | | | |
| | 704 amino acids | amino acid | | | | | |
| | Uniprot: Q5T7F0 | tail (704 aa) | | | | | |
| G | a1a2-b1b2-c | 1-856; | About 22-856; | | Δ857-923 | Yes | 67/68 |
| R | a1a2-b1b2-c | 1-856; | About 22-856 | Y297A | Δ857-923 | Yes | 69/70 |
| | | | | (VEGF, | | | |
| | | | | SEMA3A | | | |
| | | | | low) | | | |
| Z | a1a2-b1b2-c | 1-856; | About 22-856 | E319K, | Δ857-923 | Yes | 71/72 |
| | | | | D320K | | | |
| | | | | (VEGF, | | | |
| | | | | SEMA3A | | | |
| | | | | low) | | | |
| AB | a1a2-b1b2-c | 1-856; | About 22-856 | E348K, | Δ857-923 | No | 73/74 |
| | | | | S346A | | | |
| | | | | (Sema3A | | | |
| | | | | low) | | | |
| AC | a1a2-b1b2-c | 1-856; | About 22-856 | D320K | Δ857-923 | Yes | 75/76 |
| | | | | (VEGF low) | | | |
| O | a1a2-b1b2 | 1-583; | About 22-583; | | Δ584-923 | Yes | 77/78 |
| Q | a1a2-b1b2 | 1-583 | About 22-583 | Y297A | Δ584-923 | No | 79/80 |
| | | | | (VEGF, | | | |
| | | | | SEMA3A | | | |
| | | | | low) | | | |
| M | a1a2-b1 | 1-424; | About 22-424; | | Δ425-923 | Yes | 81/82 |
| P | a1a2-b1 | 1-424 | About 22-424 | Y297A | Δ425-923 | No | 83/84 |
| | | | | (VEGF, | | | |
| | | | | SEMA3A | | | |
| | | | | low) | | | |
| N | a1a2 | 1-265 | About 22-265 | | Δ266-923 | No | 85/86 |
| W | a1a2-b1-c (in part) | 1-430 and | About 22-430 | | Δ431-583; Δ796-923 | Yes | 87/88 |
| | | 584-795 | and 584-795 | | | | |
| X | a1a2-b1-c, (in part) | 1-430 and | About 22-430 | Y297A | Δ431-583; Δ857-923 | No | 89/90 |
| | | 584-856 | and 584-856 | (VEGF, | | | |
| | | | | SEMA3A | | | |
| | | | | low) | | | |
| Y | a1a2-c | 1-274 and | About 22-274 | | Δ275-583; Δ857-923 | No | 91/92 |
| | | 584-856 | and 584-856 | | | | |
| S | a1a2-b1-c | 1-430 and | About 22-430 | | Δ431-583; Δ857-923 | Yes | 93/94 |
| | | 584-856 | and 584-856 | | | | |
| AD | a1a2b1 | 1-424, | About 22-424 | D320K | Δ425-560 | No | 51/52 |
| | | 561-583 | and 561-583 | (VEGF low) | Δ584-923 | | |
| AE | a1a2b1b2 | 1-560 | About 22-560 | D320K | Δ561-923 | No | 53/54 |
| | | | | (VEGF low) | | | |
| AF | a1a2b2c | 1-280 and | About 22-429 | | Δ281-430; | No | 55/56 |
| | | 431-856 | and 431-856 | | Δ857-923 | | |
| AG | a1a2b2 | 1-280, | About 22-280, | | Δ281-430; | Yes | 57/58 |
| | | 431-583 | 431-583 | | Δ584-630; and | | |
| | | and 631-700 | and 631-700 | | Δ701-923 | | |
| AJ | a2b1b2c | 1-26, | About 22-26, | | Δ27-142; | Yes | 59/60 |
| | | 143-630 | 143-630 | | Δ631-700; | | |
| | | and 701-856 | and 701-856 | | Δ857-923 | | |
| AK | a2b1b2 | 1-26 and | About 22-26 | | Δ27-142 | Yes | 61/62 |
| | | 143-583 | and 143-583 | | Δ584-923 | | |
| AR | a2b1 | 1-26 and | About 22-26 | | Δ27-142 | Yes | 63/64 |
| | | 143-424 | and 143-424 | | Δ425-923 | | |
| AS | a2b1c | 1-26, | About 22-26, | | Δ27-142 | Yes | 65/66 |
| | | 143-430, | 143-430, | | Δ431-583 | | |
| | | and 584-856 | and 584-856 | | Δ857-923 | | |

*may vary depending on cell type/condition because of SP maturation
**numbering with reference to full length NRP1 (including SP)

In an embodiment, the NRP1 trap of the present invention comprises: (i) amino acids 1-856 (preferably in its mature form, from the aa following the signal peptide (e.g., aa 21, 22 or 28) to aa 856) of the human NRP1 polypeptide; (ii) amino acids 1 to 583 (preferably in its mature form, from the aa following the signal peptide (e.g., aa 21, 22 or 28) to aa 583) of the human NRP1 polypeptide; (iii) amino acids 1 to 424 (preferably in its mature form, from the aa following the signal peptide (e.g., aa 21, 22 or 28) to aa 424) of the human NRP1 polypeptide; (iv) amino acids 1 to 265 (preferably in its mature form, from the aa following the signal peptide (e.g., aa 21, 22 or 28) to aa 265) of the human NRP1 polypeptide; (v) amino acids 1 to 430 and 584 to 856 (preferably in its mature form, from the aa following the signal peptide (e.g., aa 21, 22 or 28) to aa 430 and aa 584 to aa 856) of the human NRP1 polypeptide; (vi) amino acids 1 to 274 and 584 to 856 (preferably in its mature form, from the aa following the signal peptide (e.g., aa 21, 22 or 28) to aa 274 and aa 584 to aa 856) of the human NRP1 polypeptide; (vii) amino acids 1 to 430 and 584 to 856 (preferably in its mature form, from the aa following the signal peptide (e.g., aa 21, 22 or 28) to aa 430 and aa 584 to aa 856) of the human NRP1 polypeptide. In embodiments, the NRP1 polypeptide comprises or consists of the amino acid sequence set forth in FIG. 17, SEQ ID NO: 44, 45, 46, 47, 95 or 96 or an allelic variant or functional variant thereof.

Given that NRP1 distinctly regulates the effects of its ligands on signal transduction and cellular responses, it may be advantageous to specifically inhibit of the activity of SEMA3A not that of the others. In a particular embodiment, the NRP1 traps of the present invention may comprise one or more mutation which reduces the ability of NRP1 to bind to for example, VEGF. Such mutation may be used to more specifically modulate the activity of NRP1 associated with the binding of SEMA3A, with fewer effects on endogenous NRP1 activities associated with other ligands.

Thus, in an embodiment, the NRP1 trap of the present invention is a polypeptide which binds to SEMA3A but not to VEGF. For example the NRP1 trap may comprise the a1 and/or a2 subdomain(s) which bind(s) to SEMA3A but not the b1 and/or b2 subdomain(s) required for VEGF binding (e.g., 1.1, Trap M, Trap N, Trap Y—see Table 2). In an embodiment, the NRP1-derived trap comprises domains a1 and a2 corresponding to amino acids 22 to 275 of the human NRP1 amino acid sequence set forth in FIG. 17. The NRP1 trap may also comprise a mutation (e.g., a deletion or substitution) which abrogates or reduces significantly the binding of VEGF to NRP1 but not that of SEMA3A to NRP1 or may preferentially bind to SEMA3A compared to VEGF (e.g., Trap Z,—see also Tables 2 and 6). Non-limiting examples of such mutation comprise a substitution at the glutamic acid at position 319 and at the aspartic acid at position 320 in NRP1 (e.g., E319K and D320K such as in Trap AC and Z).

In an embodiment, the soluble NRP1 polypeptide or functional variant or fragment thereof (i.e., NRP1 trap) comprises or consists of traps as set forth in FIGS. 17, 18, SEQ ID NO: 44, 45, or 46 or Table 2 or any functional variant thereof which binds to SEMA3A. In embodiments, the soluble NRP1 polypeptide trap comprises or consists of the extracellular domain of a polypeptide set forth in SEQ ID NO: 47, 95 or 96. In embodiments, the NRP1 trap which is used in accordance with the present invention has a binding affinity for SEMA3A which is at least 3 times that for VEGF165. In embodiments, the NRP1 trap which is used in accordance with the present invention has a binding affinity for SEMA3A which is at least 4 times that for VEGF165. In embodiments, the NRP1 trap which is used in accordance with the present invention has a binding affinity for SEMA3A which is at least 5 times that for VEGF165. In embodiments, the NRP1 trap which is used in accordance with the present invention has a binding affinity for SEMA3A which is at least 10 times that for VEGF165. In embodiments, the NRP1 trap which is used in accordance with the present invention has a binding affinity for SEMA3A which is at least 15 times that for VEGF165. In embodiments, the NRP1 trap which is used in accordance with the present invention has a binding affinity for SEMA3A which is at least 18 times that for VEGF165. In embodiments, the NRP1 trap which is used in accordance with the present invention has a binding affinity for SEMA3A which is at least 10 times that for VEGF165. In embodiments, the NRP1 trap which is used in accordance with the present invention has a binding affinity for SEMA3A which is at least 20 times that for VEGF165 (see for example Table 6).

Because the NRP1 traps of the present invention are secreted, they generally lack the transmembrane domain (e.g., corresponding to amino acids residues 860 to 883 of the NRP1 polypeptide sequences shown in FIG. 17) and the cytosolic domain (e.g., corresponding to amino acids residues 884-923 of the NRP1 polypeptide isoform 1 sequences shown in FIG. 17) found in, for example, human NRP1 isoform 1 (SEQ ID NOs: 95, 96, and FIG. 17). In embodiments, the NRP1 traps of the present invention lacks completely or partially domain c of NRP1. NRP1 isoform 1 comprises a larger c domain (see FIG. 17), while that of isoforms b and c is shorter.

As noted above, the present invention also encompasses the use of functional variants and functional fragments of the NRP1 polypeptide traps described herein in the methods described herein. Functional variants are derived from “wild-type” (native) human NRP1 polypeptides sequences (including any allelic variations naturally found in the population, i.e., allelic variants). Accordingly, as used herein, a “functional variant” or “functional fragment” refers to any NRP1 derivative having substantially the same biological activities with respect to cellular senescence as the NRP1 traps of the present invention (i.e., are capable of reducing or preventing induction of the SASP and cellular senescence). Hence, functional derivatives include but are not limited to, proteins which differ from the NRP1 polypeptide traps disclosed herein by any modifications, and/or amino acid substitutions, deletions, additions (e.g., intra-sequence insertions) or carboxyl-terminal fusions which do not significantly decrease the intended biological effects of the NRP1 traps of the present invention (e.g., inhibition or prevention of SEMA3A-mediated cellular senescence or inhibition or prevention of SEMA3A-dependent propagation of cellular senescence through the SASP and ultimately inhibition of IRE1a activation and RNAse activity, etc.). Modifications can occur anywhere including in the polypeptide backbone, (i.e., the amino acid sequence), the amino acid side chains and the amino or carboxy termini as long as the modifications do not substantially negatively affect the intended function of the NRP1 trap of the present invention (i.e., the variant is a functional variant which is capable of binding and sequestering SEMA3A polypeptide (e.g., naturally occurring human soluble NRP1 isoforms or an NRP1 trap corresponding to a polypeptide fragment of the extracellular domain of “wild-type” human NRP1 such as those exemplified in Table 2 and FIG. 18).

Table 3 provides examples of amino acids that may be modified (changed or altered) in NRP1 traps of the present invention. Preferably, the modification(s) in the functional variant (i) is a conservative substitution made in accordance with Table 3 below, (ii) corresponds to a functional allelic or polymorphic variation found in the population; or (iii) corresponds to an amino acid variation found in an ortholog of the human NRP1 polypeptide. Several orthologs of the NRP1 protein are known in the art. For example, by comparing the human NRP1 polypeptide sequence with the NRP1 polypeptide sequences from other known orthologs (e.g., mouse and rat—see FIG. 17), the person skilled in the art can easily identify the conserved residues and those which vary between species and hence can identify the amino acids that may be modified without substantial effect on the desired biological activity (e.g., inhibition or prevention of cellular senescence or inhibition or prevention of propagation of cellular senescence through the SASP). Non-limiting Examples of such amino acids are provided in Table 4.

TABLE 3
Exemplary conservative substitutions

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr; Ile |
| Phe | Met; Leu; Tyr |
| Ser | Thr |
| Thr | Ser |
| Trp | Tyr |
| Tyr | Trp; Phe |
| Val | Ile; Leu |

TABLE 4
Non-limiting examples of amino acids that may be altered in the
soluble NRP1 polypeptides/NRP1 traps of the present invention.

| WT Amino | | |
|---|---|---|
| acid* | Domain | Exemplary substitution(s) |
| V11 | a1 | Threonine |
| V15 | a1 | alanine |
| P18 | a1 | Leucine |
| N24 | a1 | Serine |
| E26 | a1 | Lysine |
| D29 | a1 | Glycine |
| S35 | a1 | Asparagine |
| D62 | a1 | Glutamic acid |
| M68 | a1 | Isoleucine |
| F90 | a1 | Isoleucine |
| N96 | a1 | Glycine |
| H98 | a1 | Arginine |
| F99 | a1 | Leucine |
| R100 | a1 | Tryptophan |
| P110 | a1 | Serine |
| T153 | a2 | Alanine |
| S155 | a2 | Threonine |
| S170 | a2 | Cysteine |
| V177 | a2 | Isoleucine |
| P196 | a2 | Glutamine |
| M204 | a2 | Valine |
| D219 | a2 | Glutamic acid |
| I242 | a2 | Valine |
| 269 | a2 | Isoleucine |
| 298 | b1 | Glycine |
| A303 | b1 | valine |
| N323 | b1 | Lysine |
| K359 | b1 | Arginine |
| I360 | b1 | Valine |
| V362 | b1 | Isoleucine |
| T371 | b1 | Serine |
| I372 | b1 | Leucine |
| P378 | b1 | Alanine |
| V379 | b1 | Isoleucine |
| L380 | b1 | Isoleucine |
| V392 | b1 | Phenylalanine, leucine |
| A393 | b1 | Glycine |
| P396 | b1 | Proline, serine |
| A409 | b1 | Valine |
| T410 | b1 | Serine |
| S449 | b2 | Alanine |
| G453 | b2 | Alanine |
| S469 | b2 | Threonine |
| A476 | b2 | Serine |
| S479 | b2 | Proline |
| I481 | b2 | Threonine |
| I487 | b2 | Valine |
| E491 | b2 | Aspartic acid |
| I498 | b2 | Valine |
| G518 | b2 | Alanine |
| M528 | b2 | Threonine |
| A553 | b2 | Alanine |
| P555 | b2 | Serine, threonine |
| A556 | b2 | Proline |
| G572 | b2 | Serine |
| A587 | c | Valine |
| L599 | c | Proline |
| D601 | c | Histidine |
| V634 | c | Isoleucine |
| N667 | c | Serine |
| 669 | c | Alanine |
| K672 | c | Arginine |
| S674 | c | Arginine |
| N717 | c | Serine |
| R741 | c | Histidine |
| A755 | c | Valine |
| I756 | c | Valine |
| S805 | c | Proline |
| A813 | c | Threonine |
| P820 | c | Threonine |
| G835 | c | deletion |
| E838 | c | Lysine |
| E854 | c | Aspartic acid |
| T916 | cytoplasmic | Proline |
| T919 | cytoplasmic | Asparagine |
| A179 | a2 | Valine |

*with ref. to FIG. 17 and SEQ ID NO: 96

Other functional variants of NRP1 traps of the present invention may be made by introducing one or more mutations corresponding to natural (allelic) variants detected in the population. These natural variants can be readily identified using well-known publicly available databases such as through the NCBI, GeneCard; HOMIM and Ensembl websites.

In embodiments, the functional variant of the NRP1 trap of the present invention comprises or consists of amino acids 1-857 of SEQ ID NO: 47 or a functional fragment thereof. In embodiments, the functional variant comprises one or more conservative amino acid substitutions located at one or more amino acid positions set forth in Table 4. In embodiments, the amino acid substitution is as set forth in Table 4. In embodiments,

The soluble NRP1 polypeptide trap or functional fragment or variant (allelic variant) thereof of the present invention may comprise one or more additional polypeptide domain(s) to increase synthesis, purification, stability and/or bioavailability. For example, NRP1 traps of the present invention may include a FC domain (or part thereof such as the human FC domain) or a purification tag (e.g., a 6×-histidine tag). Such additional polypeptide domain(s) may be linked directly or indirectly (through a linker) to the soluble NRP1 polypeptide or functional fragment or variant thereof. In an embodiment the one or more additional domain is at the C-terminal end of the NRP1 polypeptide trap. In an embodiment the one or more additional domain is at the N-terminal end of the NRP1 polypeptide trap.

The soluble NRP1 polypeptide or functional variant or fragment thereof of the present invention may optionally include one or more polypeptide linkers. Such linkers may be used to link one or more additional polypeptide domain(s) to the soluble polypeptide of the present invention (e.g., a polypeptide domain which increases the stability of the polypeptide in vivo and/or a domain which facilitates purification of the polypeptide). Linker sequence may optionally include peptidase or protease cleavage sites which may be used to remove one or more polypeptide fragments or domains (e.g., removal of purification tag prior to in vivo administration of the soluble NRP1 polypeptides or functional variant or fragment thereof). One non-limiting example of a linker or domain which enables cleavage of the polypeptide and removal of, for example, polypeptide domain(s) (e.g., 6×his tag purification domain) includes a polypeptide comprising a TEV protease cleavage site (e.g., EXXYXQIG or S, where \ denotes the cleavage site, SEQ ID NOs: 97 and 98). Many other TEV cleavage sites are known and many other protease/peptidase cleavage sites are known to the skilled person and may be introduced in the polypeptides of the present invention to optionally remove one or more polypeptide domains or fragments.

Polypeptide linkers may also be used to replace totally or partially domains which are normally found in the wild-type NRP1 polypeptide but which are absent in the soluble NRP1 polypeptide or functional variant or fragment thereof of the present invention. For example, in the NRP1 traps of the present invention, synthetic linkers may replace totally or partially subdomains a1, a2, b1, b2 and c. The length of the linker may correspond to the entire length of the domain removed or to a portion of it. Such linkers may increase protein folding, stability or binding to NRP1 ligands. Non-limiting examples of NRP1 traps comprising linkers are described in WO2016/033699, which is incorporated herein by reference. One non-limiting example of a useful polypeptide linker is a polyarginine polypeptide. Other linkers are known in the art and may be used in accordance with the present invention.

Thus, the present invention further provides soluble NRP1 polypeptides or functional variants or fragments thereof, nucleic acids encoding the soluble NRP1 polypeptides or functional variants or fragments thereof, vectors comprising the nucleic acids and host cells comprising the nucleic acids or vectors.

c. Inhibition of SEMA3A Expression

Various approaches are available for decreasing SEMA3A expression and thus SEMA3A-mediated cellular senescence. Non-limiting example includes the use of small hairpin shRNA (RNAi), antisense, ribozymes, TAL effectors targeting the SEMA3A promoter, CRISPR/Cas9/Cpf1 systems or the like.

Expression of shRNAs or similar inhibitory RNAs in cells can be obtained by delivery of plasmids or through viral (e.g., lentiviral vector) or bacterial vectors. Non-limiting examples of shRNAs that may be used to inhibit SEMA3A expression are provided in Table 9 (see Example 11).

Therefore, in alternative embodiments, the invention provides antisense, shRNA molecules (iRNA) and ribozymes for exogenous administration to effect the degradation and/or inhibition of the translation of mRNA of interest. Preferably, the antisense, shRNA molecules and ribozymes target mammalian (preferably human) SEMA3A. An exemplary method of screening for antisense and ribozyme nucleic acids that may be used to provide such molecules as SEMA3A inhibitors of the invention is disclosed in U.S. Pat. No. 5,932,435.

In a further embodiment, expression of a nucleic acid encoding a polypeptide of interest (SEMA3A or NRP1), or a fragment thereof, may be inhibited or prevented using RNA interference (RNAi) technology, a type of post-transcriptional gene silencing. Examples of therapeutic antisense oligonucleotide applications and additional information about antisense molecules, shRNAs and RNAi technologies are provided above in relation to the inhibition of IRE1α and apply to the same extent to the inhibition of SEMA3A expression.

Accordingly, in an embodiment expression of a nucleic acid encoding a polypeptide of interest (SEMA3A or NRP1), or a fragment thereof, may be inhibited by introducing into or generating within a cell an siRNA or siRNA-like molecule corresponding to a nucleic acid encoding a polypeptide of interest (e.g. SEMA3A), or a fragment thereof, or to an nucleic acid homologous thereto.

### (ii) Methods of Promoting Cellular Senescence by Increasing SEMA3A Activity

Cellular senescence, including autocrine and paracrine senescence can be promoted or induced by stimulating or increasing SEMA3A activity (i.e., SEMA3A cellular signaling). SEMA3A activity can be increased by a number of approaches including by increasing the expression of SEMA3A in a cell or by contacting a cell with a SEMA3A polypeptide or functional fragment or variant thereof.

Methods of promoting cellular senescence may be useful in diseases and conditions where senescence has beneficial effects such as tissue repair, cancer, renal fibrosis, wound healing, liver fibrosis, myocardial infarction cardiac fibrosis, atherosclerosis and pulmonary hypertension.

### 6. Modulation of Lipid Parameters

Applicants have found that the NRP1 gene is involved in the control of lipid metabolism (fat uptake/storage/accumulation) and that administration of a soluble NRP1 polypeptide or fragment thereof (e.g., NRP1 trap) significantly reduces diet-induced weight gain and improves lipid parameters, with benefits (or with concomitant positive effects) on blood glucose levels and insulin sensitivity.

Accordingly, in a further aspect, the present invention provides a method of altering a lipid parameter in a subject comprising modulating the expression and/or activity of the NRP1 gene and/or its associated NRP1 protein (e.g., transmembrane isoform 1). In a particular aspect, the method comprises administering to the subject a compound or composition which reduces or inhibits the expression and/or activity of the NRP1 protein. In embodiments, the method comprises administering to the subject (a) a soluble NRP1 polypeptide or fragment thereof (e.g., an NRP1 trap); (b) an NRP1 antibody; or (c) a composition comprising (a) and/or (b) together with a pharmaceutically acceptable carrier.

As used herein, the expression “disease or condition associated with fat accumulation” comprises any disease or condition which is caused by fat accumulation or considered comorbidity to fat accumulation (e.g., diet-induced overweight or obesity). A comorbidity is a medical condition whose prevalence highly increases (i.e., the risk of suffering from such additional disease or condition increases) in the presence of the original condition (e.g., fat accumulation; overweight or obesity). The term can indicate either a condition existing simultaneously with the original metabolic condition (e.g., fat accumulation) or a risk of developing such comorbid condition. The disease or condition associated with fat accumulation is said to be caused by, or otherwise related to fat accumulation in the subject. Diseases and conditions associated with fat accumulation include: high BMI; obesity; metabolic syndrome; NAFLD; cardiovascular diseases (CVD; heart diseases (e.g., congestive heart failure); coronary artery disease (hypercholesterolemia and atherosclerosis) pulmonary embolism, dyslipidemia and stroke); hypertension and Type II Diabetes mellitus (TIIDM). In embodiments, the fat accumulation corresponds to a BMI greater than or equal to 25 kg/m². In another embodiment, the fat accumulation corresponds to a BMI greater than or equal to 30 kg/m².

Body composition parameters associated with fat accumulation are well known in the art. Such body composition parameters include visceral fat area (VFA), body mass index (BMI), waist to hip ratio (WHR); waist-to-height ratio, waist circumference (WC); arm circumference (AC), conicity index, per cent body fat (PBF), triceps skin fold, subscapular skin fold, white adipose tissue (WAT) level; and brown adipose (BAT) tissue level.

Modulation of NRP1-mediated lipid metabolism can be achieved using naturally occurring soluble NRP1 polypeptides or synthetic (e.g., recombinantly produced or chemically synthesized) NRP1 polypeptides described herein.

### 7. Compositions/Formulations

The active ingredient(s) (e.g., one or more SASP inhibitor including one or more IRE1α inhibitors, an inhibitor of SEMA3A (e.g., an NRP1 trap), etc.) can be provided in a pharmaceutical composition. Pharmaceutical compositions for use in accordance with the present invention may be formulated in a conventional manner using one or more physiologically acceptable carriers comprising excipients and auxiliaries which facilitate processing of the active compounds into preparations which can be used pharmaceutically. The pharmaceutical compositions can include other medicinal or pharmaceutical agents, carriers, adjuvants, such as preserving, stabilizing, wetting or emulsifying agents, solution promoters, salts for regulating the osmotic pressure, and/or buffers. Proper formulation is dependent upon the route of administration chosen. For injection, the agents of the invention may be formulated in aqueous solutions, preferably in physiologically compatible buffers such as Hanks's solution, Ringer's solution, or physiological saline buffer. Methods well known in the art for making formulations can be found in, for example, Remington: The Science and Practice of Pharmacy, (20th ed.) ed. A. R. Gennaro A R., 2000, Lippencott Williams & Wilkins.

In embodiments, the compositions of the present invention are formulated for delivery to the eye e.g., eye drops or ocular injections. For ocular administration, the compounds can be formulated readily by combining the active compounds with pharmaceutically acceptable carriers suitable for ocular administration, as well known in the art. In embodiments, the carrier is a carrier which is not naturally found in mixtures with the compounds/agents/inhibitors of the present invention (i.e., a non-naturally occurring carrier).

For example, the pharmaceutical compositions can be formulated for topical administration, intravitreal administration, intracameral administration, subconjunctival administration, subtenon administration, retrobulbar administration, posterior juxtascleral administration, or a combination thereof. In some embodiments, the pharmaceutical compositions are formulated for topical administration. In some embodiments, the pharmaceutical compositions are formulated for intravitreal administration.

Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Alternatively, other delivery systems for pharmaceutical compounds may be employed. Liposomes and emulsions are well known examples of delivery vehicles. Particularly useful delivery system for periocular drug delivery (e.g., in the prevention and/or treatment or ocular diseases such as retinal diseases) include the transscleral absorption pathway which is considered one of the safest means of achieving consistent therapeutic drug concentrations in the inner coat of the posterior segment.

Effective dosage. Pharmaceutical compositions suitable for use in the present invention include compositions wherein the active ingredient(s) is/are contained in an effective amount to achieve the intended purpose. More specifically, a therapeutically effective amount means an amount effective to prevent development of or to alleviate at least one of the existing symptoms of the subject being treated. Determination of the effective amounts is well within the capability of those skilled in the art.

In embodiments, the effective dose of the compound(s) used in accordance with the present invention inhibits cellular senescence or propagation of cellular senescence (through the SASP) sufficiently to reduce or prevent at least one symptom or physiological effect associated with cellular senescence in diseases and conditions described herein (e.g., ocular vascular diseases and other diseases and conditions described herein). Certain compounds which have such activity can be identified by in vitro assays that determine the dose-dependent inhibition of SASP and/or IRE1α.

Alternatively, in other embodiments, the effective dose of the compound(s) used in accordance with the present invention is sufficient to induce or increases the SASP and cause cellular senescence.

For any compound used in the method of the invention, the therapeutically effective dose can be estimated initially from cellular assays. For example, a dose can be formulated in cellular and animal models to achieve a circulating concentration range that includes the IC50 as determined in cellular assays (i e., the concentration of the test compound which achieves a half-maximal inhibition of the cellular signaling function of SASP and/or IRE1α, (usually in response to inflammatory mediators such as Il-1β or other activating stimulus such as hypoxia, ischemia, cellular stress, ER stress).

A therapeutically effective amount refers to that amount of the compound that results in amelioration of symptoms in a subject. Similarly, a prophylactically effective amount refers to the amount necessary to prevent or delay symptoms in a patient (e.g., vascular hyperpermeability, spotted and/or blurry vision, pericytes loss, macular edema, retinal swelling, blood retinal barrier leakage, pathological neovascularization, reduced vascular repair, etc.). Toxicity and therapeutic efficacy of such compounds can be determined by standard pharmaceutical procedures in cell cultures or experimental animals, e.g., determining the maximum tolerated dose (MTD) and the ED (effective dose for 50% maximal response). The dose ratio between toxic and therapeutic effects is the therapeutic index and it can be expressed as the ratio between MTD and ED50. Compounds which exhibit high therapeutic indices are preferred. The exact formulation, route of administration and dosage can be chosen by the individual physician in view of the patient's condition.

Dosage amount and interval may be adjusted individually to provide levels of the active compound which are sufficient to maintain the desired modulating effects, or minimal effective concentration (MEC). The MEC will vary for each compound but can be estimated from in vitro data; e. g. the concentration necessary to achieve substantial inhibition of SASP and/or IRE1α expression or activity (e.g., secretion of cytokines, proteases and growth factors associated with the SASP, ribonuclease activity activation and processing of XBP1s) Dosages necessary to achieve the MEC will depend on individual characteristics and route of administration.

### Definitions

In order to provide clear and consistent understanding of the terms in the instant application, the following additional definitions are provided.

The articles “a,” “an” and “the” are used herein to refer to one or to more than one (i.e., to at least one) of the grammatical object of the article.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps and are used interchangeably with, the phrases “including but not limited to” and “comprising but not limited to”.

For the recitation of numeric ranges herein, each intervening number there between with the same degree of precision is explicitly contemplated. For example, for the range of 18-20, the numbers 18, 19 and 20 are explicitly contemplated, and for the range 6.0-7.0, the number 6.0, 6.1, 6.2, 6.3, 6.4, 6.5, 6.6, 6.7, 6.8, 6.9, and 7.0 are explicitly contemplated. The terms “such as” are used herein to mean, and is used interchangeably with, the phrase “such as but not limited to”.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

Practice of the methods, as well as preparation and use of the products and compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Green and Sambrook MOLECULAR CLONING: A LABORATORY MANUAL, 4th edition, Cold Spring Harbor Laboratory Press, 2014; Ausubel et al., CURRENT PROTOCOLS IN MOLECULAR BIOLOGY, John Wiley & Sons, New York, 2003 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

The terms “treat/treating/treatment” and “prevent/preventing/prevention” as used herein, refer to eliciting the desired biological response, i.e., a therapeutic and prophylactic effect, respectively. In accordance with the subject invention, the therapeutic effect comprises an amelioration of symptoms, and/or a reduction in the severity of the disease or condition (e.g., vascular eye disease), following administration of a pharmaceutical composition or compound (e.g., SASP inhibitor) of the present invention.

As used herein the term “preventing” or “prevention” in reference to diseases or conditions associated with senescence is meant to refer to a reduction in the progression or a delayed onset of at least one symptom associated with the disease or condition or one feature of cellular senescence.

Described herein are methods for modulating cellular senescence. As used herein, “cellular senescence” refers to a condition of a cell in which the cell is viable and metabolically active but has either lost the ability to proliferate or remains part of the tissue architecture but is unable to function/communicate properly with the rest of the tissue (i.e., it becomes dormant). Cellular senescence may increase with age or exposure to factors that induce DNA damage, such as mutation or chromosomal damage, or that induces a DNA damage response or disruption of chromatin structure resulting in changes in gene expression, such as genes associated with SASP. Senescence is thought to be a result of DNA or chromosomal insults including telomere shortening, chromosomal aneuploidy, DNA strand breaks, DNA chemical modification (e.g. alkylation), or triggering of a DNA damage response (DDR). Cellular senescence may be caused by, for example, ischemia, oncogene activation (through DDR) DNA damaging compounds such as chemotherapeutic agents, or DNA damaging radiation such as ionizing and UV radiation. Senescence may be caused by various other treatment regimes, such as corticoid treatment, anti-retroviral treatment, treatment with PPARy agonists, treatment with xanthine oxidase inhibitors, treatment with bisphosphonates, treatment with antiprotozoal agents, and treatment with inflammatory agents. Senescence may also be caused by metabolic imbalance such as increased caloric intake, insulin resistance, type II diabetes, hyperinsulinemia, high fat diets, high protein diets, ER-stress response (UPR response, as demonstrated herein) and alterations in gut microbiota associated with these diseases. Senescent cells develop a distinctive secretome including metalloproteases, growth factors and inflammatory cytokines, a process named senescence-associated secretory phenotype (SASP) (37), which can propagate senescence to the surrounding tissue in a cell autonomous and non-cell-autonomous (paracrine) fashion (38-40). Thus, paracrine cellular senescence may be induced in cells as a consequence of the senescence-associated secretory phenotype (SASP). Paracrine senescence refers to a state of heightened secretion of proteins, such as pro-inflammatory cytokines (SASP), by senescent cells.

In various embodiments, the cellular senescence is caused by: (a) ischemia; (b) ageing of the cell; (c) DNA damage to the cell; (d) contact with a chemotherapeutic agent; (e) Irradiating the cell with DNA damaging radiation; (f) contacting the cell with an anti-retroviral agent; (g) contacting the cell with a proinflammatory agent; (h) contacting the cell with a DNA damaging agent; (i) contacting the cell with an agent that disrupts chromatin structure; (j) telomere erosion; (k) hypoxia; (l) oncogene activation; (m) telomere dysfunction and (o) any combination of at least two of (a) to (m).

Cells that have undergone cellular senescence may exhibit one or more of the following characteristics: growth arrest, formation of γ-H2AX (a phosphorylated form of the histone variant H2AX) nuclear foci; a rise in the level of pI6INK4A; a rise in the expression level of p21 (Cipl/Waf1); increased activity of senescence-associated β-galactosidase; production of senescence-associated heterochromatic foci (SAHF); loss of proliferation; trimethylation of histone 3 lysine 9 (H3K9me3); endoplasmic reticulum stress and induction of the unfolded protein response (UPR); increased level and/or activation of tp53; increased number and size of PML nuclear bodies; activation of IRE1α; increased glucose consumption; increased expression and/or secretion of pro-inflammatory cytokines, proteases and growth factors, of the “senescence-associated secretory phenotype” (SASP) (which may include, but is not limited to, Pai1, IL-6, IL-7, IL-lα, IL-lβ, IL-8, TGF-β1, MCP-2, MCP4, MIP-la, MIP-3a, eotaxin-3, GM-CSF, MIF, EGF, FGF, HGF, VEGF, KGF, PIGH, NGF, MMP1, MMP3, MMP12, MMP13, MMP14, IGFBP2, IGFBP3, IGFBP4, IGFBP6, IGFBP7, fibronectin, cathepsin B, TIMP-2); lack of expression of Ki67; enlarged and flatten cell morphology; persistent DNA damage response (DDR) signaling; and formation of DNA segments with chromatin alterations reinforcing senescence (DNA-SCARS), which are nuclear foci which may contain DDR proteins such as phospho-ATM and ATR substrates. Cells that have undergone cellular senescence typically have increased levels of p16INK4a expression relative to the level of P16INK4a expression in cells that have not undergone cellular senescence. Also, cells that have undergone cellular senescence typically have increased levels of SA-β-Gal activity relative to that of cells that have not undergone cellular senescence.

As used herein, a “senescent cell” or a “cell harboring a senescent phenotype” refers to a cell having at least one of the following features: (i) growth arrest, (ii) enlarged and flatten cell morphology, (iii) DNA damage foci in the nucleus, (iv) secretion of growth factors proteases, cytokines and other factors defined as the senescence-associated secretory phenotypes (SASP) (e.g., PAI1, TNFAAIP2, IGFBP3, VIM, CDKN1A, FN1, CDKN2B, RRAS, IRF7, HSPA2, TES, CTGF, CCND1, ESM1, THBS1, S100A11, RAB31, IGFBP5, IL6, IL1β, TGFβ1, VEGFA, TP53), (v) senescence-associated β-galactosidase (SA-β-gal) activity (which partly reflects the increase in lysosomal mass), (vi) expression of the tumor suppressor p16INK4a (which may activate pRB and cause the formation of senescence-associated heterochromatin foci (SAHF)); (vii) SEMA3A expression; (viii) IRE1a activation (S724 phosphorylation) and increase splicing of XBP1s and/or (ix) increase expression of γH2AX, PML and/or p53 activation. In embodiments, a “senescent cell” is a cell having at least the features: (i), (ii) and/or (ii), (v), (vi) and (ix). In embodiments, the senescence is secondary to cellular ischemia. In embodiments, the senescence is paracrine senescence. In embodiments, the senescence is senescence after differentiation. In embodiments, the senescence is premature senescence. In embodiments, the premature senescence in characterized by an increase in the expression and/or RNAse activity of IRE1α. In embodiments, the senescence is retinal senescence. In embodiments, the senescence is microglial senescence. In embodiments, the senescence is characterized by (i) increased expression and/or activity of P16INK4a, Tp53, IRE1a, Cdkn1a Cdkn2a and/or senescence associated beta-gal activity; (ii) expression of γH2Ax and/or PML; and/or (iii) the expression of the senescence-associated secretory phenotype (SASP). In embodiments, the SASP comprises the secretion of IL-1β, IL-6, Pai1, TGFβ1, IRE1a and/or VEGF-.a. In embodiments, the above-mentioned SASP is secondary to cellular ischemia.

In embodiments, the above-noted cell is a terminally differentiated cell. In embodiments, the cell is a neuron, a microglial cell, a myeloid cell, a monocyte, a macrophage, an endothelial cell, a hepatic cell, a fat cell, a fibroblast, and/or retinal cell. In embodiments, the cell has suffered from cellular ischemia. In embodiments, the cell is a retinal ganglion cell. In embodiments, the cell is a retinal ganglion neuron. In embodiments, the cell is a vascular cell. In embodiments, the cell is a vascular endothelial cell. In embodiments, the cell is an avascular cell (i.e., it is located in an avascular area/region). In embodiments, the cell is an hepatic stellate cell. In embodiments, the cell is a microvascular endothelial cell. In particular embodiments, the cell is not an ocular cell. In particular embodiments, the cell is not a retinal cell. In embodiments, the cell is a mammalian cell. In embodiments, the cell is a human cell.

As used herein, “cellular ischemia” refers to a restriction in oxygen and/or nutrients (e.g., glucose) supply needed for cellular metabolism (to keep tissue alive) as well as inadequate removal of metabolic wastes. It includes local anemia in a given part of a body sometimes resulting from congestion (such as vasoconstriction, thrombosis or embolism). Ischemia can be partial (poor perfusion) or total. Ischemia is generally caused by problems with blood vessels (e.g., embolism, thrombosis (e.g., of an atherosclerotic artery), trauma, aneurysm, cardiomyopathies, hypoglycemia, radiotherapy, hypotension, anemia etc.) with resultant damage to or dysfunction of tissue.

The term “effective amount,” as applied to the compound(s), biologics and pharmaceutical compositions described herein, means the quantity necessary to render the desired therapeutic result. For example, an effective amount is a level effective to treat, cure, or alleviate the symptoms of a disorder for which the therapeutic compound, biologic or composition is being administered. Amounts effective for the particular therapeutic goal sought will depend upon a variety of factors including the disorder being treated and its severity and/or stage of development/progression; the bioavailability, and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and like factors well known to one of skill in the relevant scientific art. Some variation in dosage can occur depending upon the condition of the subject being treated, and the physician or other individual administering treatment will, in any event, determine the appropriate dose for an individual patient.

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid/polynucleotide sequence is “homologous” to another sequence if the two sequences are substantially identical and the functional activity of the sequences is conserved (as used herein, the term ‘homologous’ does not infer evolutionary relatedness). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90%, 95%, 98% or 99% identical. As used herein, a given percentage of homology between sequences denotes the degree of sequence identity in optimally aligned sequences. An “unrelated” or “non-homologous” sequence shares less than 40% identity, though preferably less than about 25% identity, with any of the nucleic acids and polypeptides disclosed herein.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, at least 95%, at least 98%, or at least 99%. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, WI, U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridisation to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). Generally, stringent conditions are selected to be about 5° C. lower than the thermal melting point for the specific sequence at a defined ionic strength and pH. For example, In embodiments, the compound of the present invention is an antisense/RNAi or shRNA that hybridizes to an NRP1 or SEMA3A nucleic acid sequence (preferably a human sequence).

The present invention is illustrated in further details by the following non-limiting examples.

### EXAMPLE 1

### Retinal Ischemia Triggers Cellular Senescence

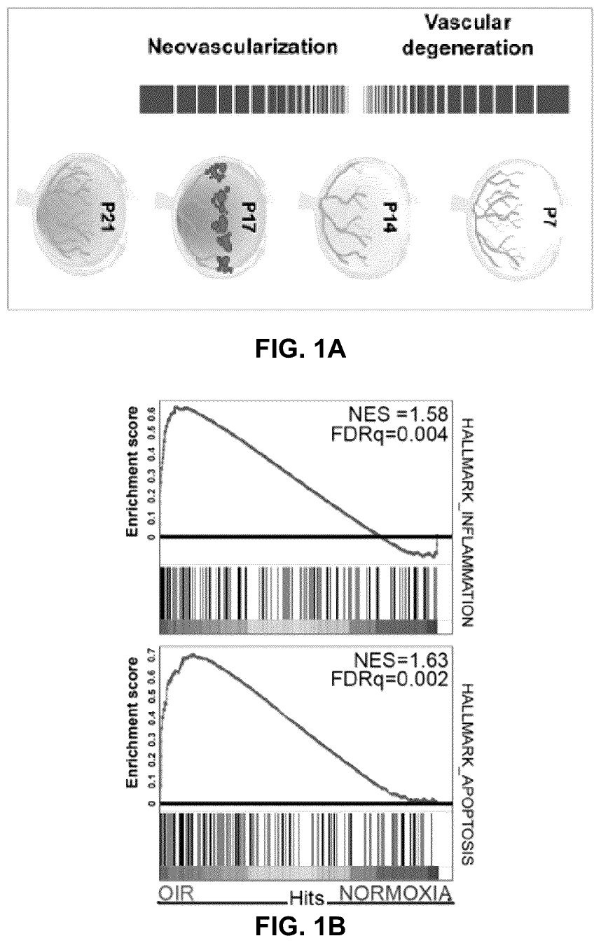

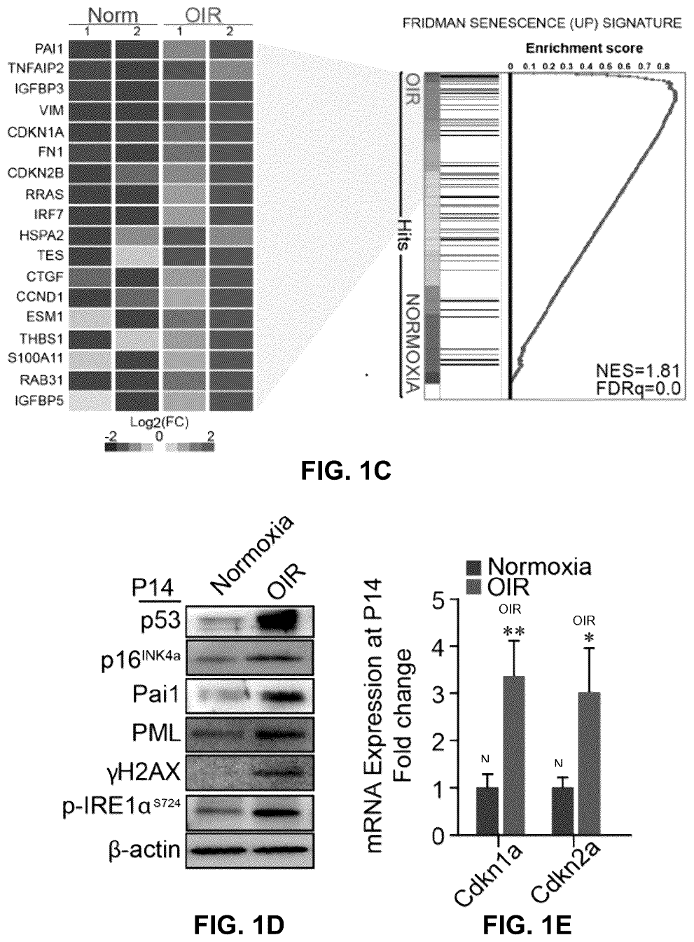

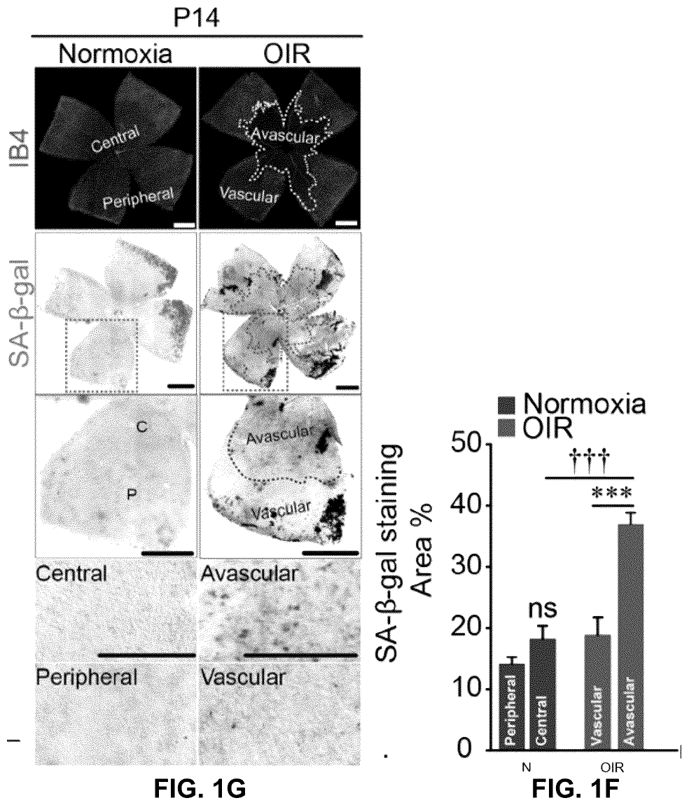

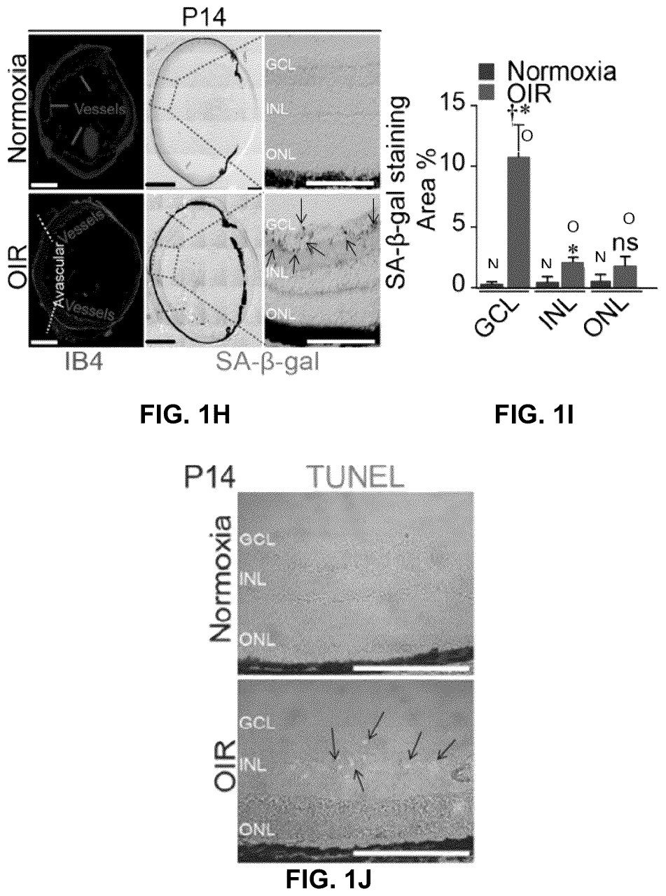

In order to elucidate the cellular processes triggered subsequent to vascular degeneration in ischemic retinopathies, we subjected mouse pups to a model of oxygen-induced retinopathy (OIR) that yields avascular neural zones similar to those observed in DR and ROP (27). Mouse pups were exposed to 75% oxygen from postnatal day (P) 7 to 12 to induce vaso-obliteration and returned to ambient air where maximal pre-retinal neovascularization is reached at P17 (27) (FIG. 1A). We then performed an unbiased transcriptomic analysis by high throughput RNA-sequencing of retinas at P14 OIR (when pathological pre-retinal angiogenesis commences) and carried out Gene Set Enrichment Analysis (GSEA) to identify defined gene expression patterns that were modulated. As expected, we found a strong positive correlation in clusters of inflammation (normalized enrichment score (NES)=1.58; false discovery rate (FDRq)=0.004) and apoptosis (NES=1.63; FDRq=0.002) (FIG. 1B). Given the vastly post-mitotic nature of the neural retina, we also unexpectedly noticed a significant enrichment in the Fridman senescence signature (28) cluster (NES=1.81; FDRq=0.0) (FIG. 1C).

Cellular senescence is a permanent state of cell cycle arrest in which a cell remains viable and metabolically active (29). In a predominantly post-mitotic tissue such as the retina, senescence may be triggered through a DNA damage response or stimulation of tumor suppressor networks reported to be activated in ischemic retinas (30). A senescent state may thus protect retinal cells from low metabolic supply associated with ischemia and help escape hypoxia-associated cell death. Induction of senescence during OIR was further supported by upregulation of classical senescence-associated proteins, such as p53, p16^{INK4a}, Pai1, PML, γH2AX and activation of the ER-stress effector inositol requiring enzyme 1α (IRE1α), which has been suggested to promote cellular senescence (31) (FIG. 1D), and significantly increased transcript levels of cyclin-dependent kinase inhibitors (CDKi) Cdkn1a and Cdkn2a in OIR retinas (FIG. 1E).

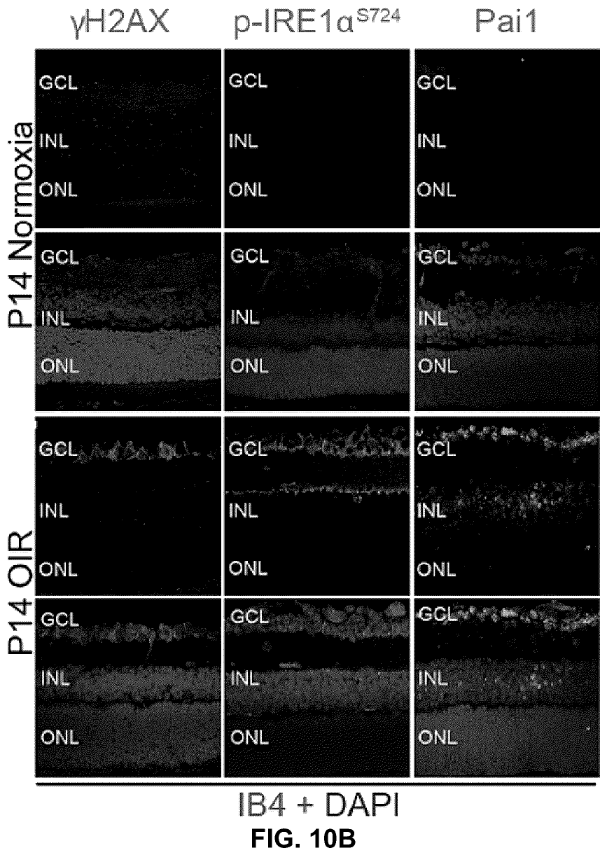

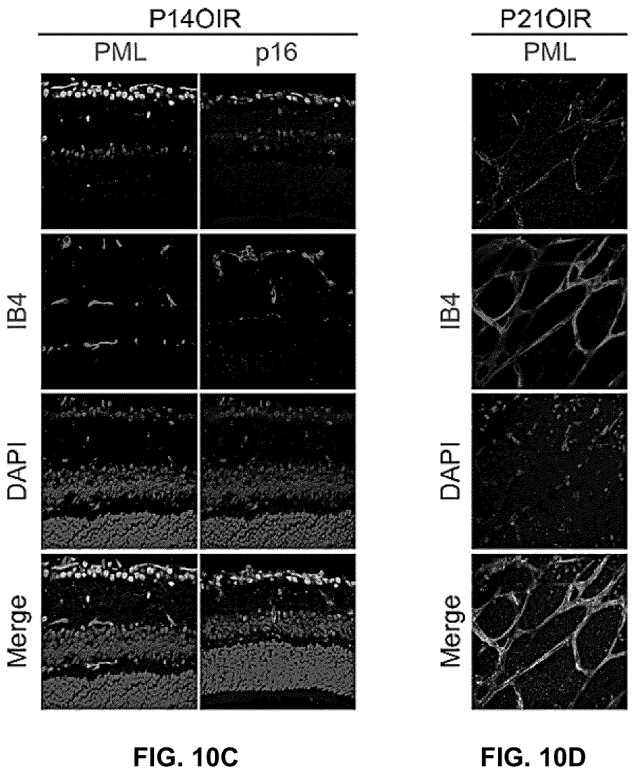

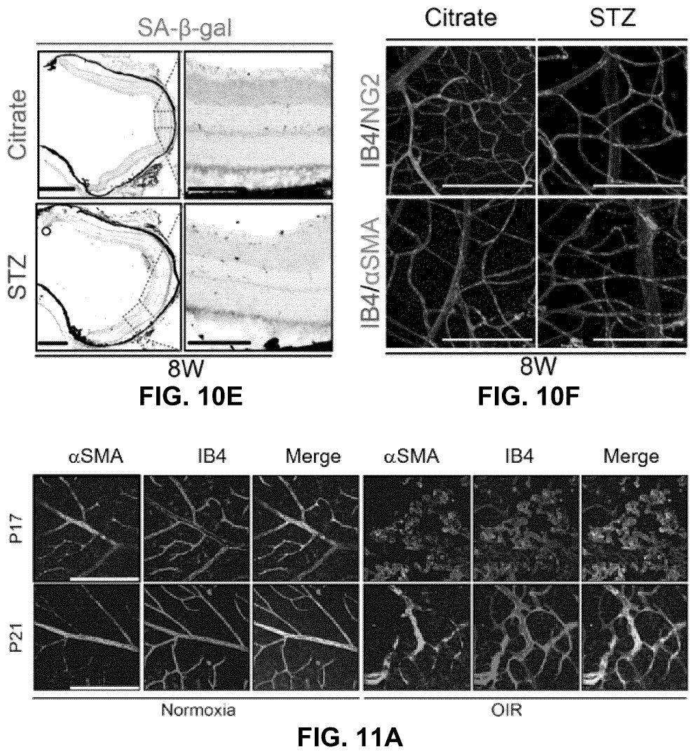

To determine which cells were triggering a program of senescence during OIR, we performed senescence-associated β-galactosidase (SA-β-gal) staining on retinal flatmounts at P14. Counterstaining with Isolectin B4 (IB4) revealed that senescent cells resided predominantly in avascular zones (36.99% of cells are SA-β-gal⁺) compared to vascularized areas (18.79%; P<0.0001). Low numbers of SA-β-gal⁺ cells were also found in control normoxic retinas (FIGS. 1F and G). In line, analysis of sagittal retinal sections revealed significantly elevated SA-β-gal staining in devascularized areas of the retinal ganglion cell layer (GCL) (10.77% vs 0.3%; P=0.0167) and to a lesser extent in the inner nuclear layer (INL) (2.61% vs 0.45%; P=0.0342) (FIGS. 1H and I). Both layers are intimately associated with the inner retinal vasculature that degenerates in ischemic retinopathies. Because of the hypoxic/oxidative and inflammatory nature of the ischemic retina (32) and GSEA of apoptotic genes in FIG. 1B, we further sought to establish which cells were undergoing apoptotic death during OIR. Terminal deoxynucleotidyl transferase-mediated biotinylated dUTP nick end labeling (TUNEL) revealed a predominance of apoptotic cells in the INL (FIG. 1J) and mostly in the periphery (FIG. 10A). Taken together (FIGS. 1J), these data reveal a mutually exclusive pattern of retinal cellular senescence and apoptosis, where cells of the GCL associated with the central zone primarily adopt a senescent phenotype while cells of INL are more susceptible to apoptosis.

### EXAMPLE 2

### Retinopathy Triggers a Senescence-Associated Secretory Phenotype Which Propagates Cellular Senescence

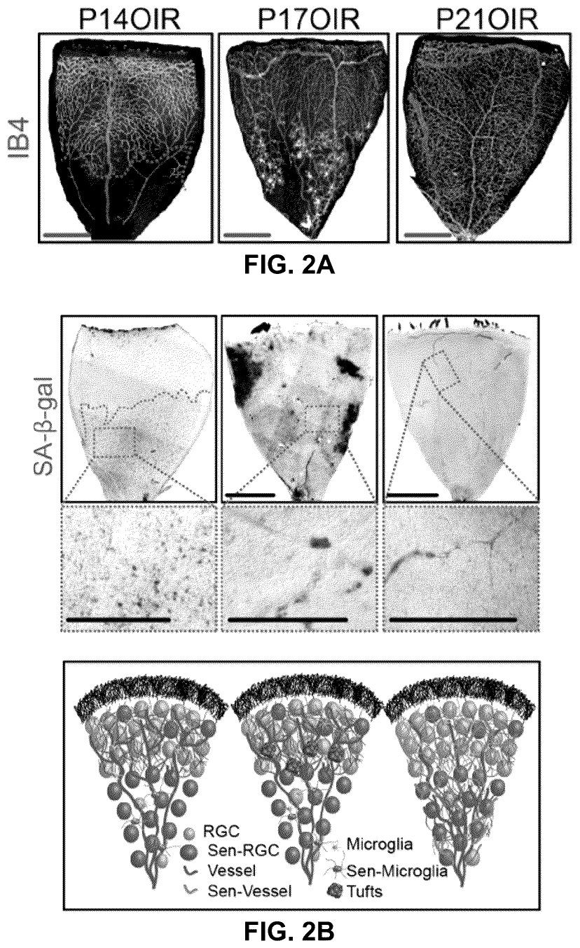

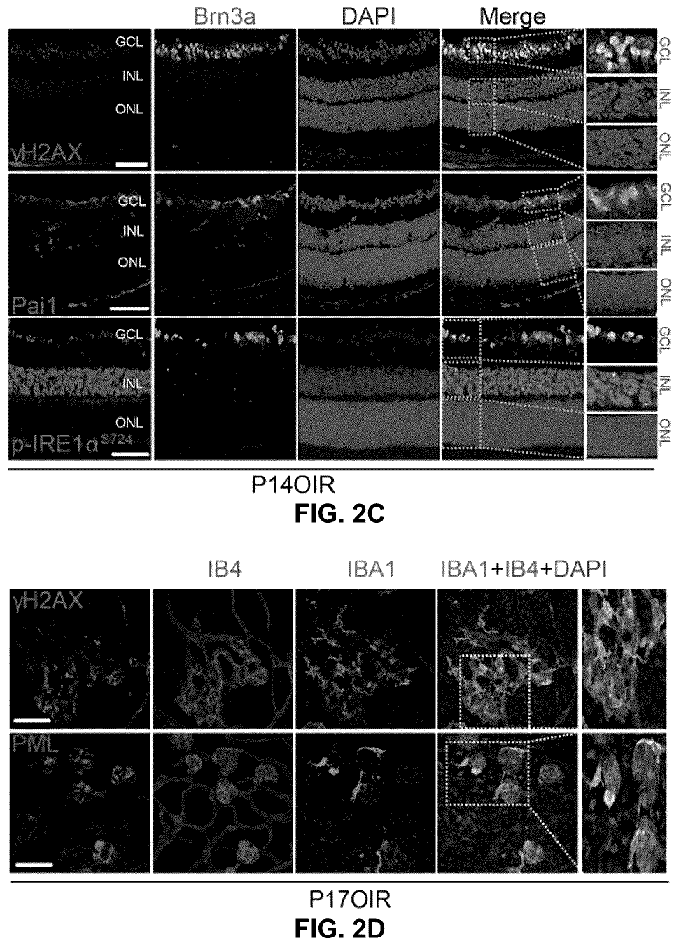

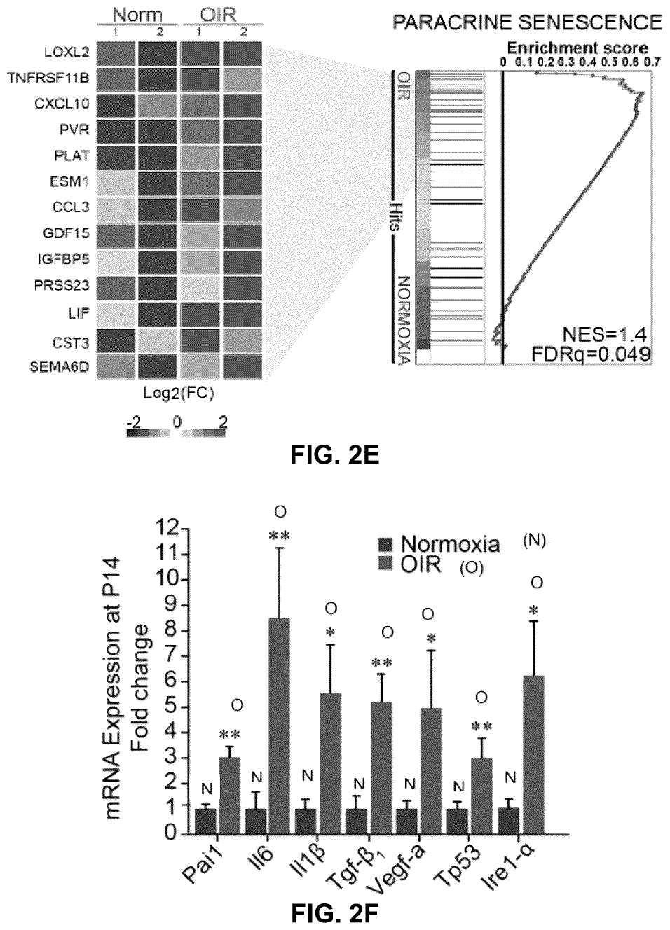

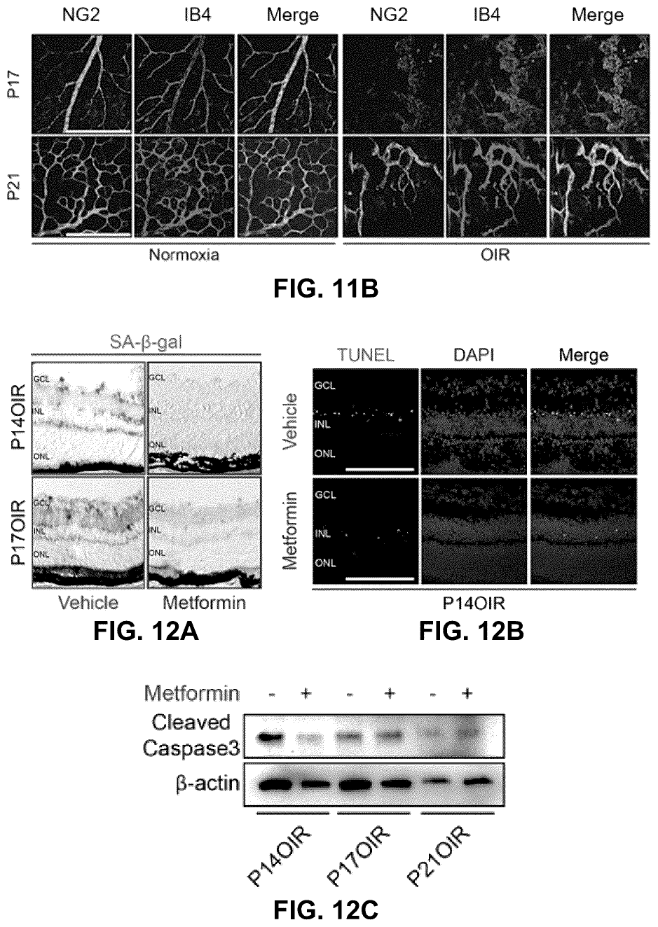

The SASP typically reinforces senescence in autocrine and paracrine manners, heightens inflammation and has detrimental effects on tissue microenvironment (34). We interrogated whether the cellular senescence initially observed in the GCL at P14 during OIR (FIG. 1J) propagates to other cell populations of the retina. Initial SA-β-gal staining at P14 in OIR is concentrated in avascular areas (FIGS. 2A and B left panels) and centered on retinal ganglion neurons (RGCs) as substantiated by colocalization of Brn3a+ RGCs with markers of senescence (γH2AX, Pai1 and p-IRE1α^{S724}) (FIG. 2C). At this early time point, there is absence of senescence-associated labeling in vessels (FIGS. 10B and C). At P17, during maximal neovascularization, cellular senescence localizes to pathological vascular tufts (FIGS. 2A and B middle panels) and retinal microglia as evidenced by co-labeling of senescence markers γH2AX or PML with IB4 (vessels) and IBA1 (microglia) (FIG. 2D). This vasculature is tortuous and leaky (35) and less stable with less pericyte coverage (FIGS. 11A, B) (36). By P21 of OIR, when retinal vasculature has regenerated, SA-β-gal staining is predominantly confined to vascular cells (FIGS. 2A and B right panels, FIG. 10D). At this later time point, pericyte coverage is re-instated (FIGS. 11A, B). In addition, we observed SA-β-gal stained cells in the GCL of mice at 8 weeks of a streptozotocin-induced model of Type I Diabetes Mellitus (FIGS. 10E, F). The presence of retinal senescence in the STZ model is particularly important given that the OIR model is conducted in mouse pups and angiogenic responses diverge according to age (37). These finding support the presence of cellular senescence in a wide range of ocular vasculopathies. Given that the OIR model is a well-known and established model for ocular vascular pathologies, further studies were conducted in this model. Nevertheless, we observed a transient accumulation of senescent cells in different subcellular populations of the retina in different models of retinopathies. The dynamically evolving pattern of cellular senescence as a function of disease progression supports paracrine senescence.

Senescent cells develop a distinctive secretome including metalloproteases, growth factors and inflammatory cytokines, a process named senescence-associated secretory phenotype (SASP) (39), which can propagate senescence to the surrounding tissue in a cell autonomous and non-cell-autonomous (paracrine) fashion (40-42). Heatmap and GSEA of OIR retinas also identified a positive correlation between retinal ischemia and paracrine senescence-associated genes (NES=1.4; FDRq=0.049) (FIG. 2E). Elevated expression of SASP-associated cytokines in OIR was confirmed by RT-qPCR for Pai1, Il6, Il1β, Tgf-β1, Vegf-a, as well as IRE1α, and the tumor suppressor Tp53 (FIG. 2F), suggesting that several cytokines central to pathological angiogenesis may originate from senescent retinal cells.

### EXAMPLE 3

### Secretion of SEMA3A by Senescent Cells Drives Paracrine Senescence

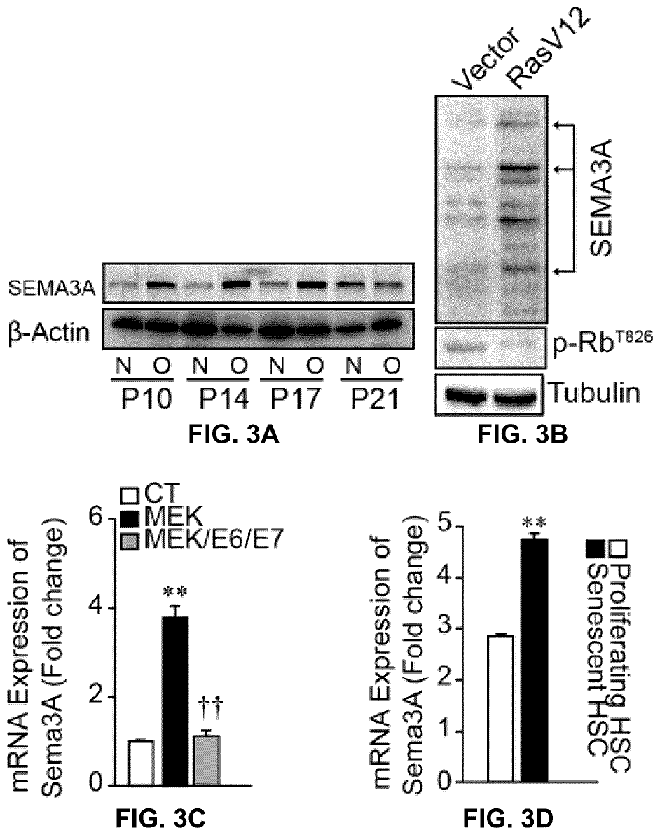

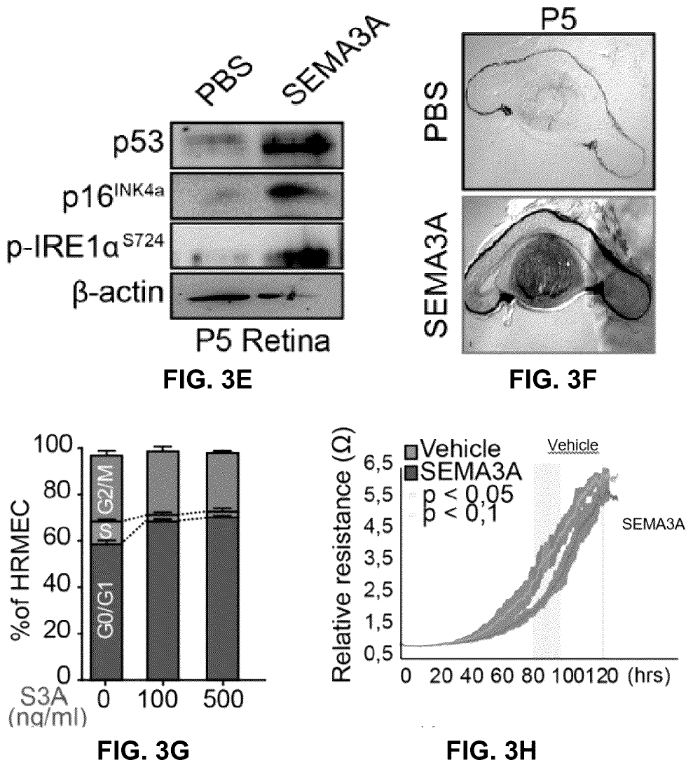

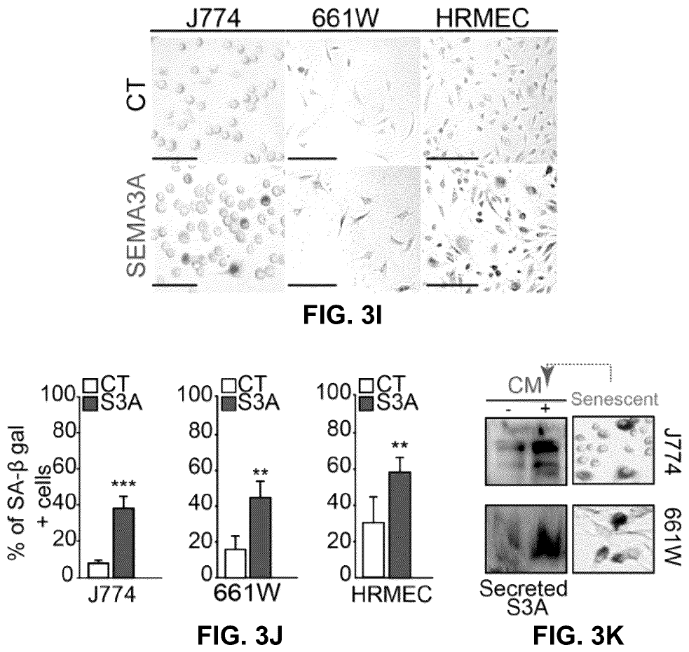

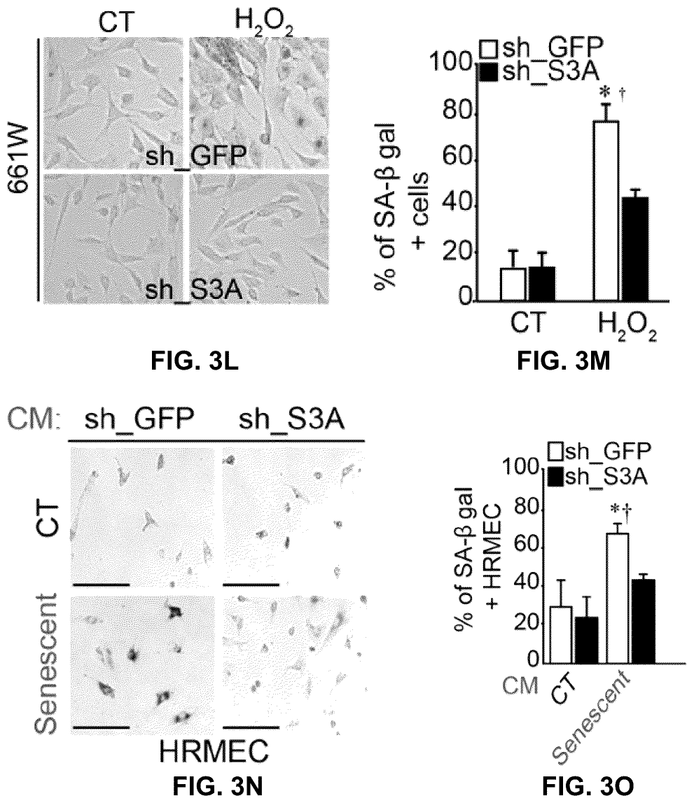

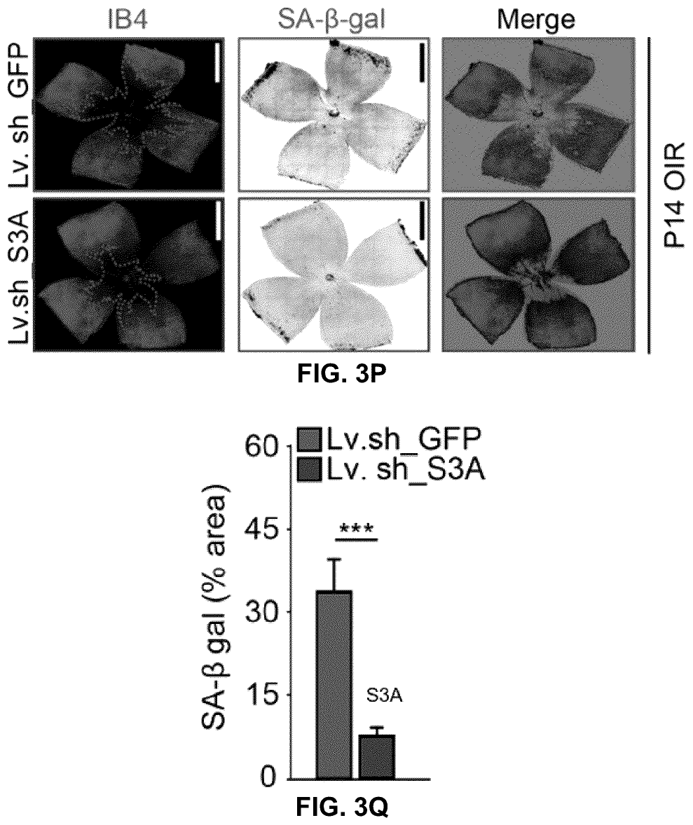

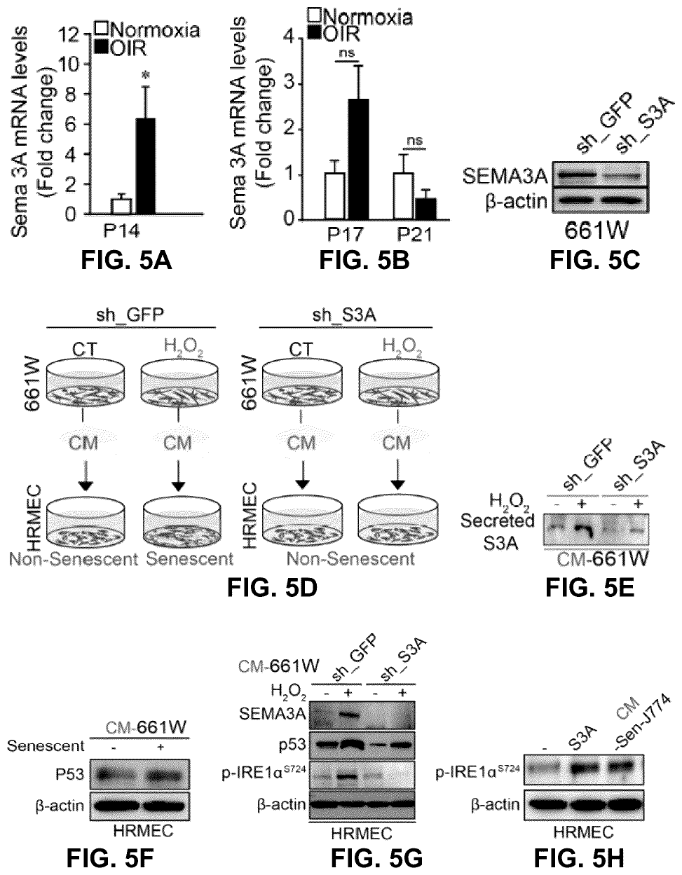

Given the spread of cellular senescence, we sought to identify factors that drive this process in OIR. An effector molecule associated with retinal ischemia that has been suggested to perturb cell cycle (43) is the classical guidance molecule Semaphorin 3A (SEMA3A). SEMA3A is induced throughout the vaso-obliterative and vaso-proliferative phases of OIR (44) and is secreted by hypoxic neurons to deviate regenerating blood vessels and metabolic supply towards less affected regions of the retina (44, 45). Given that expression of SEMA3A is temporally consistent with markers associated with senescent cells during progression of retinopathy (FIG. 3A, FIGS. 5A and B), we questioned whether SEMA3A contributed to propagating paracrine senescence.

First, evidence for a potential contribution of SEMA3A to paracrine senescence stemmed from observations that senescence-inducing oncogenes such as RasV12 (FIG. 3B) and MEK (FIG. 3C) trigger production of SEMA3A, as do stress-induced senescent human hepatic stellate cells (HSC) (FIG. 3D). In addition, intravitreal injection of recombinant SEMA3A in P5 pups induced a marked increase of p53, p16INK4a and IRE1α (FIG. 3E) accompanied by a marked augmentation of SA-β-gal staining (FIG. 3F).

Ultimately, exposure of HRMECs to SEMA3A for 7 days (mimicking the first week of OIR) increased cell cycle arrest in G0/G1 while significantly reducing the S phase (FIG. 3G) and stunted normal endothelial cell growth as measured by electric cell impedance sensing (ECIS) (FIG. 3H).

A role for SEMA3A in driving senescence is further substantiated by direct exposure of cells to recombinant SEMA3A, which induces senescence, in macrophage-like J774 cells (P<0.0001), in a cell line of retinal neurons (661 W photoreceptors used to model retinal neurons) (46) (P<0.001) and in HRMECs (P<0.001) (FIGS. 3I and J). In addition, H₂O₂-driven-senescence which mimics the oxidative environment in retinopathy (3), triggered secretion of SEMA3A in cell lines that model populations entering senescence in retinopathy such as macrophage-like J774 cells (J774) and neuronal 661W cells (FIG. 3K).

To verify the potential involvement of neuron-derived SEMA3A in driving paracrine endothelial cell senescence, we exposed Human retinal microvascular endothelial cells (HRMECs) to conditioned medium (CM) from senescent 661 W cells (FIGS. 3L and M) in which Sema3a was silenced by lentiviral (Lv) vectors carrying small hairpin RNAs (shRNAs) (44, 47). Efficacy of this approach was demonstrated (FIGS. 5C-E). CM from the senescent retinal neuron cell line, effectively propagated senescence in a paracrine fashion given induction of SA-β-gal expression in 68% of HRMECs (P<0.005) (FIGS. 3N left panels, O), highlighting that factors secreted by senescent cells have the propensity to stimulate senescence in neighboring cells. Conversely, CM from Sema3A-deficient senescent retinal neuron precursor cells triggered significantly less paracrine senescence in HRMEC cells (P<0.005) (FIGS. 3N right panel, O). In parallel, incubation of HRMECs with conditioned medium (CM) from senescent neuron precursors was sufficient to activate p53 in a SEMA3A-dependent manner (FIGS. 5F and G). Interestingly, downregulating SEMA3A by intravitreal administration of Lv.sh_Sema3a at P12 of OIR significantly diminished the number of senescent SA-β-gal positive retinal cells by 75%, at P14 underscoring the critical contribution of SEMA3A to senescence during OIR (FIGS. 3P and Q). Together, these data provide evidence for production of SEMA3A during cellular senescence and its contribution in propagating paracrine senescence.

### EXAMPLE 4

### Enrichment of ER-Stress Transcripts in Retinopathy

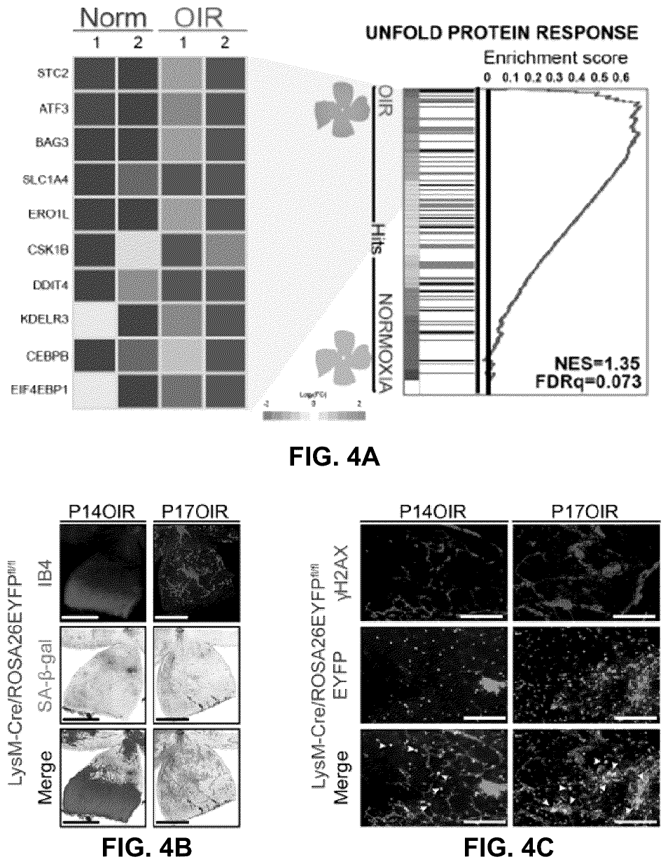

Pathways of the unfolded protein response (UPR) triggered under conditions of ER-stress can provide cells with adaptive mechanisms to survive during metabolic imbalances such as ischemia (48, 49). As supported by Applicant's findings, activation of ER-stress may help drive premature senescence. Transcriptomic analysis of retinas subjected to OIR revealed significant GSEA enrichment in transcripts related to the UPR (NES=1.41; FDRq=0.047) (FIG. 4A). Given that SEMA3A was shown to activate the IRE1a branch of the UPR (FIG. 3E and FIG. 5H), we interrogated on the contribution of IRE1α to premature senescence in ischemic retinas.

During ischemic retinopathy, there is a substantial implication of microglia and infiltration of myeloid cells that express microglial markers. We crossed myeloid-driver LysM-Cre mice with ROSA26EYFP^{fl/fl} and observed SA-β-gal staining in avascular zones (FIG. 4B) rich in EFYP+ myeloid/microglial cells (FIG. 4C). EFYP+ microglia also stained with senescence-associated DNA damage marker γH2AX and preferentially localized to the vascular/avascular border of P14 OIR retinas and to sites of pathological angiogenesis (tufts) at P17 (FIG. 4C).

### EXAMPLE 5

### SEMA3A Activates IRE1α and the RNASE Activity of IRE1α Contributes to Senescence

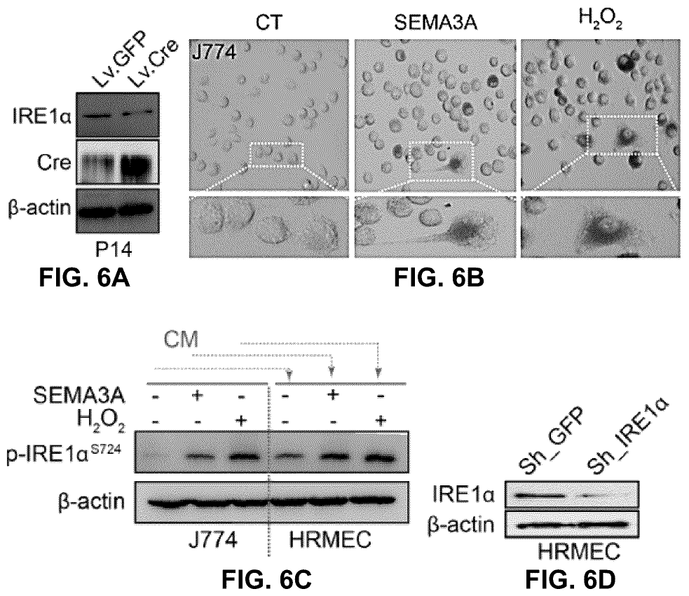

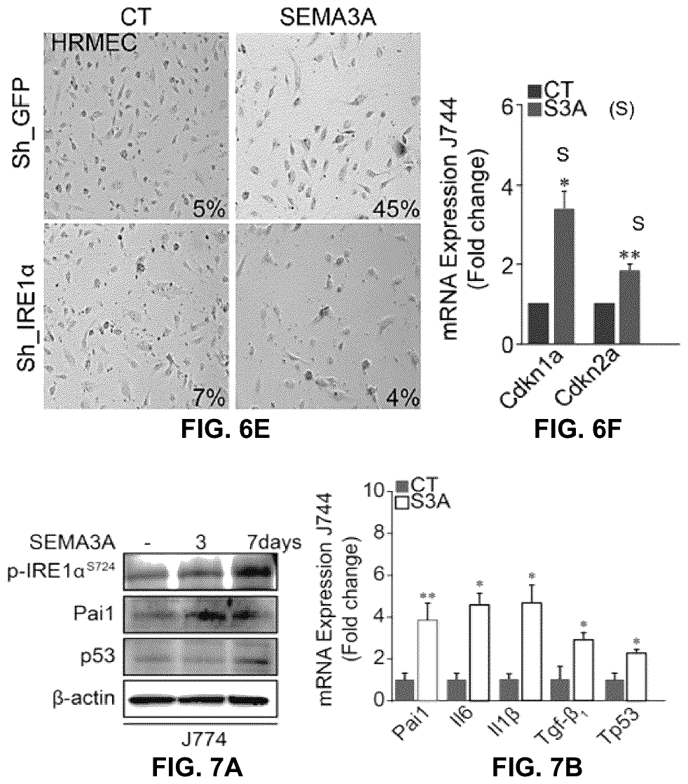

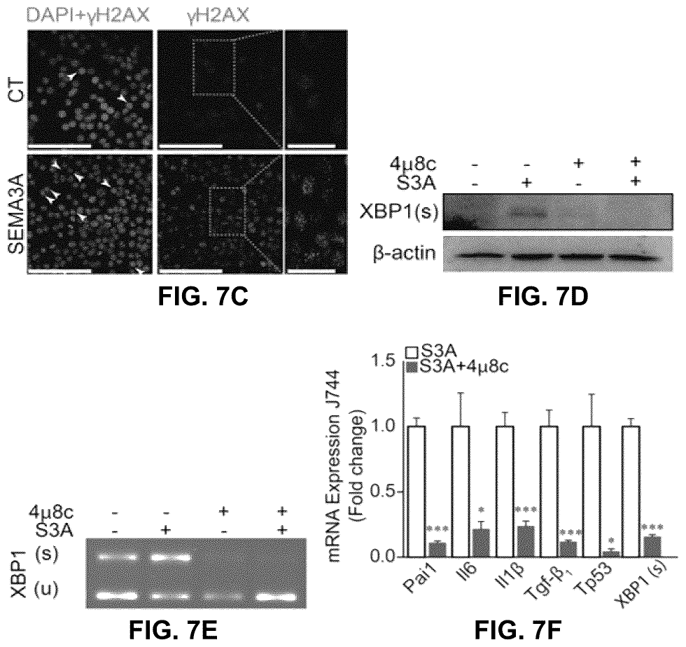

IRE1α is a type I transmembrane protein that possesses both a serine/threonine kinase domain and a distinct endoribonuclease domain on its cytosolic terminus (54, 55). Through its RNase activity, also termed IRE1α-dependent decay (RIDD), it preferentially targets mRNAs encoding proteins that traverse the ER-Golgi secretory pathway (56). In light of SEMA3A driving senescence through IRE1α (FIGS. 2, 3), we next investigated which catalytic arm was accountable for this physiological response. Given that we established that myeloid cells become senescent with retinal ischemia, we used J774 macrophage/monocyte cells and confirmed that sustained exposure to SEMA3A activated IRE1α (FIG. 7A), induced senescence (FIG. 6B) and drove expression of a panel of genes known to be critical for promoting and reinforcing the senescent state, such as Pai1, Il6, IL1β, TGF-β and Tp53 (FIG. 7B). In addition, SEMA3A promoted senescence-associated DNA-damage foci expressing γH2AX (FIG. 7C) that are hallmarks of cellular senescence (57). Similarly, shRNA-mediated knockdown of IRE1α in endothelial cells prevented SEMA3A-driven senescence (FIGS. 6D and E).

To determine whether SEMA3A-driven senescence was occurring through IRE1α's kinase or RNAse activity, the selective cell-permeable coumarin o-hydroxyaldehyde pharmacological inhibitor of IRE1α's endoribonuclease activity, 4μ8c, was used. Exposure to 4μ8c (FIG. 7D) prevented SEMA3A-induced growth arrest and abrogated SEMA3A-induced senescence (data not shown). In support, 4μ8c also prevented SEMA3A-mediated splicing and activation of the IRE1α effector X-box Binding protein-1 (XBP1) (FIGS. 7D and E). Ultimately, pharmacological inhibition of the endoribonuclease activity of IRE1α inhibited production of specific senescence-associated genes Vegf-a, Tgf-β1, Il-1β, Il-6, Pai-1, Tp53 while there is no effect on Tnf-α or IRE1α itself (FIG. 7F). These data highlight the importance of the endoribonuclease activity of IRE1α in triggering senescence.

### EXAMPLE 6

### Metformin Abrogates the SASP and Pathological Retinal Angiogenesis

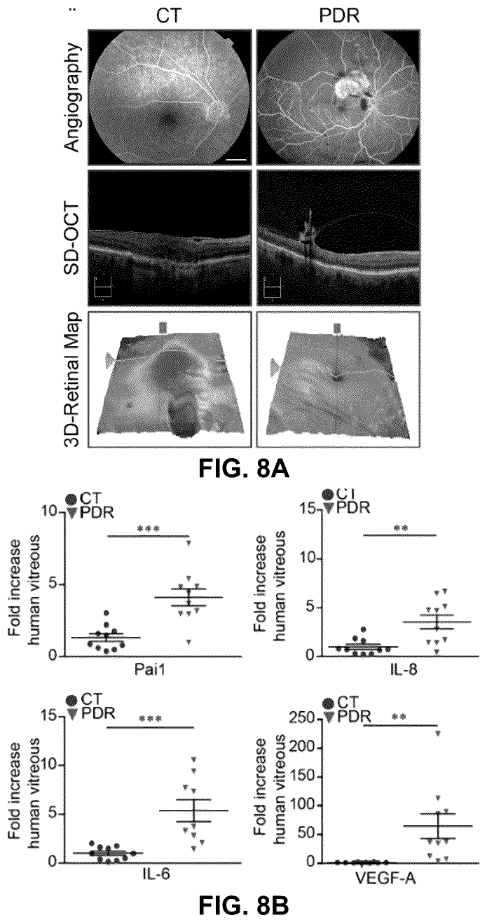

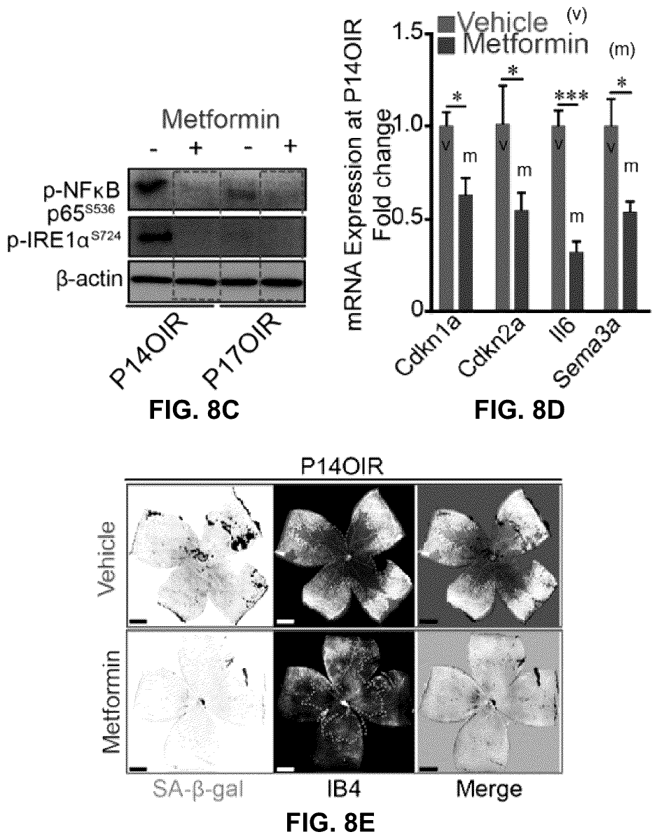

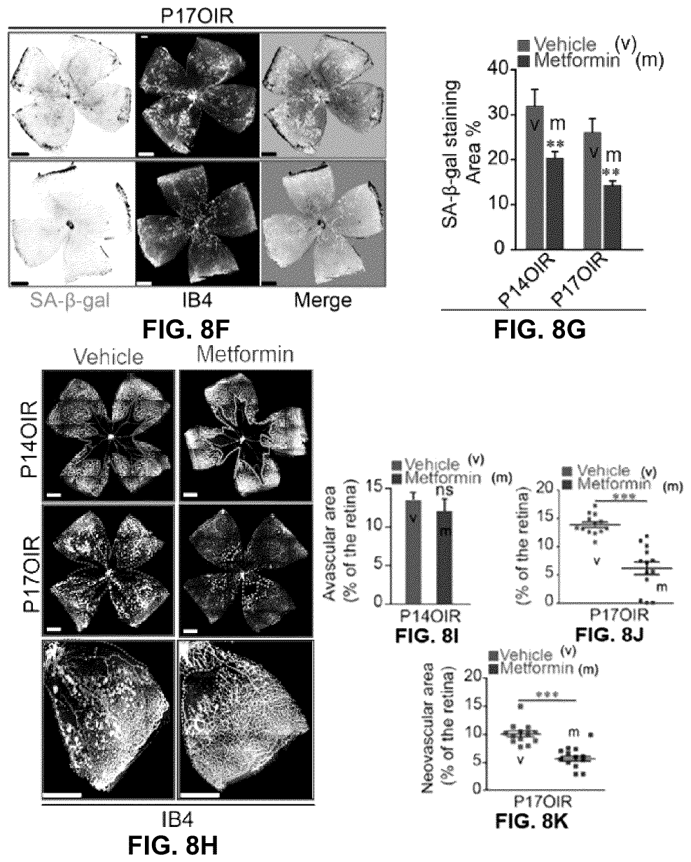

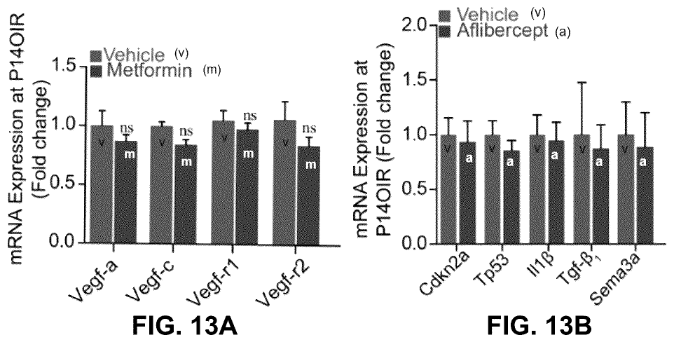

To establish the clinical relevance of therapeutic inhibition of the SASP and paracrine senescence in ischemic retinopathy, we assessed levels of key SASP proteins in the vitreous of patients suffering from active proliferative diabetic retinopathy (PDR). Angiography and spectral-domain optical coherence tomography (SD-OCT) were performed, and three-dimensional (3D) retinal maps were generated to evaluate the extent of retinal damage (FIG. 8A). Undiluted vitreous was obtained from patients with PDR and control patients with nonvascular pathology such as epiretinal membrane and macular hole that showed only non-diabetes-related retinal damage. Detailed characteristics of patients are included in Table 1. Evaluation of vitreal SASP proteins by multiplex magnetic bead-based immunoassays revealed significant increases in senescence-associated factors Pai-1 (P=0.0004) IL-6 (P=0.001), IL-8 (P=0.0037) and VEGF-A (P=0.0085) in patients with PDR (FIG. 8B). Given the association between paracrine senescence and retinopathy (FIGS. 1C and 3E), we sought to therapeutically modulate the SASP and assess outcome on pathological retinal angiogenesis. In this regard, the widely used biguanide anti-diabetic drug metformin has been reported to reduce the SASP without interfering with the growth arrest program (58). A single intravitreal injection of metformin at P12 attenuated NF-κB and IRE1α activation in mouse retinas subjected to OIR (FIG. 8C). This lead to a significant decrease in IL6, Cdkn1a, Cdkn2a and Sema3A as determined by RT-qPCR (FIG. 8D) and translated into a significant decrease in SA-β-gal at P14 (P=0.0086) (FIGS. 8E and G and FIG. 12A) and P17 (P=0.0036) (FIGS. 8F and G and FIG. 12A). Components of the VEGF signaling pathway were not affected (FIG. 13A). We elected to perform intravitreal injections of metformin given that systemic administration interfered with mouse weight gain and hence could be a confounding factor (32).

TABLE 5
Clinical characteristics of patients having undergone vitreous biopsy.

| | Sex | | |
|---|---|---|---|
| | female (F) | | |
| Sample | Male (M) | Age | Patient condition |
| C1 | M | 82 | Macular Hole (MH) |
| 2 | F | 62 | Epiretinal membrane (ERM) |
| 3 | F | 69 | ERM-control / pseudo TM |
| C4 | M | 75 | MH-Cataract |
| C5 | M | 77 | Retinal Detachment |
| C6 | M | 69 | ERM |
| C7 | M | 68 | ERM |
| C8 | M | 81 | ERM |
| C9 | M | 70 | ERM |
| C10 | F | 65 | MH |
| P1 | F | 78 | Proliferative Diabetic Retinopathy (PDR) |
| P2 | F | 72 | PDR |
| P3 | F | 69 | PDR |
| P4 | M | 36 | PDR |
| P5 | F | 70 | PDR |
| P6 | F | 74 | PDR |
| P7 | F | 67 | PDR |
| P8 | M | 69 | PDR |
| P9 | F | 70 | PDR |
| P10 | M | 45 | PDR |

We next determined if treatment with metformin and subsequent inhibition of the SASP would result in increased apoptosis. TUNEL staining revealed that treatment with metformin lowered the number of apoptotic cells in the INL layer when compared to vehicle-treated retinas without aggravating apoptosis in cells of the GCL (FIG. 12B). Findings were confirmed by Western blotting of retinas for cleaved caspase-3 during different stages of retinopathy (FIG. 12C). Ultimately, intravitreal injection of metformin enhanced vascular regeneration more than 2-fold as assessed at P17 (P<0.0001) (FIGS. 8H and J) and suppressed pathological neovascularization by half (P<0.0001) (FIGS. 8H, K). It is important to note that administering metformin via systemic paths did not show any benefits on pathological retinal angiogenesis underscoring the need for local intravitreal administration. Taken together, these data support the therapeutic inhibition of the SASP with biguanides such as metformin in the treatment of pathological ocular angiogenesis (pathological neovascularization).

### EXAMPLE 7

### VEGF Trap-Eye Abrogates Pathological Retinal Angiogenesis Yet Does Not Increase Vascular Regeneration or Reduce Senescence

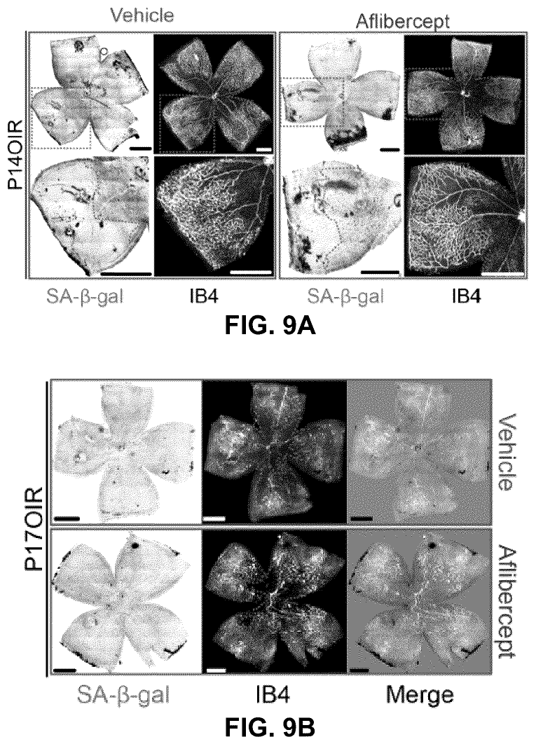

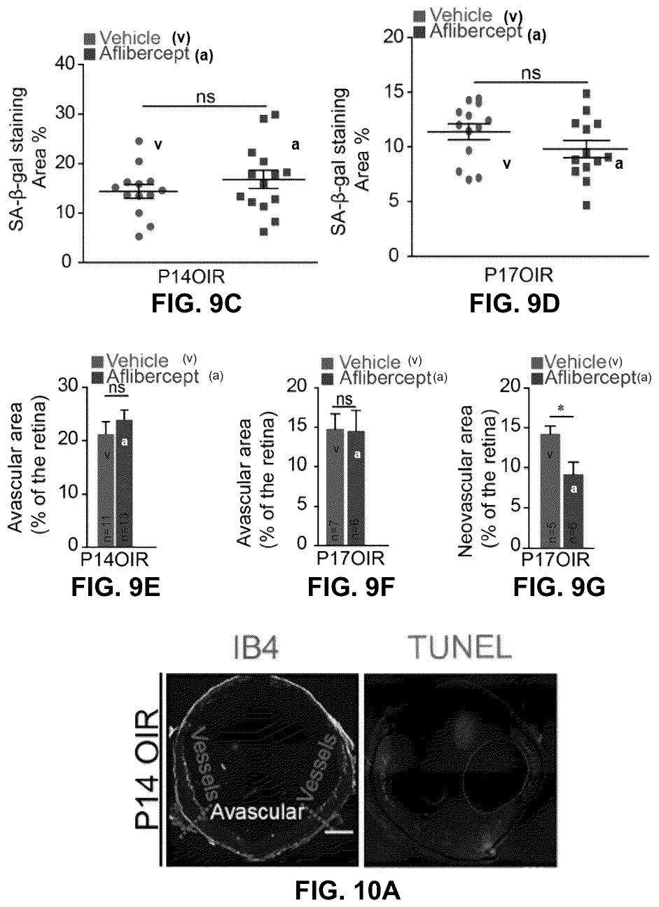

We next determined if currently used anti-VEGF treatments such as VEGF trap-eye (Aflibercept) (59, 60) influenced retinal senescence during retinopathy (60). Aflibercept is a recombinant fusion protein made-up of the extracellular domains of human VEGF receptors 1 and 2 and an Fc portion. As such, it binds at least VEGF-A and Placental Growth factor (PLGF) (59). Intravitreal injection of Aflibercept at P12 of OIR did not significantly influence SA-β-gal staining at P14 (P=0.3087) (FIGS. 9A, B) or P17 (P=0.1580) (FIGS. 9C, D). Interestingly, in contrast to treatment with metformin which reduces the SASP and augments vascular regeneration, Aflibercept does not modulate rates of vascular regeneration at P14 (P=0.4897) (FIGS. 9A, C) or P17 (P=0.9502,) (FIGS. 8B, D), nor drivers of senescence (FIG. 13B). As expected, intravitreal injection of Aflibercept lead to a marked decrease in neovascularization at P17 OIR (P=0.0207) (FIG. 9G), (*P<0.001). Hence, treatment with Aflibercept does not relieve retinal ischemia or senescence nor enhance retinal vascular repair, and only directly blocks pathological neovascularization. Together, these data further strengthen the link between cellular senescence and ischemia-driven pathological angiogenesis.

### EXAMPLE 8

### Preparation of Soluble SEMA3A Neutralizing Traps

High affinity traps to inhibit/neutralize SEMA3A were generated. These traps were derived from Neuropilin 1 (NRP1) and were optionally coupled to 6×-His tag or FC proteins (see FIG. 18 and Table 2). Various variants comprising either the entire NRP1 extracellular domain or functional variants capable of maintaining SEMA3A binding were generated. Traps containing a b1 domain (which binds to VEGF) and including a neutralizing VEGF₁₆₅ mutation were generated. The traps were shown to be highly expressed and secreted in transformed human cells. Simple purification and formulation protocols were developed to produce trap samples for structure-activity relationships (SAR) and in vivo efficacies studies.

### Methods

Cell culture and material. The human Neuropilin 1 (GenBank™ accession NM_003873, SEQ ID NO: 66) was acquired from Origene Inc. The Origen clone comprises a conservative mutation at amino acid 140 which changes the leucine for an isoleucine. The 293T (ATCC) cells were grown in Dulbecco's modified Eagle's medium supplemented with 10% fetal calf serum. The pFUSE-hIgG1-Fc1 vector was purchased from InvivoGen Inc.

Cloning. The extracellular domain of Neuropilin-1 (residues 1-856), or portions of it, were PCR amplified from Origene clone RC217035 using the Phusion™ high fidelity polymerase (New England Biolabs) and cloned in the EcoR1-BgIII of pFUSE-hIgG1-Fc1 in frame with the human FC-1 coding sequence. Constructs coding for the soluble versions of the traps were generated by inserting a sequence coding for a TEV protease cleavage site followed by 6×His residues and a stop codon upstream of the FC coding portion of the corresponding FC constructs. Additional deletions (b1, b1b2) or VEGF165 binding mutations (e.g., Y297A) were introduced using the Q5 site directed mutagenesis kit (NEB). All constructs sequences were verified by Sanger sequencing (Genome Quebec).

Evaluation of traps' expression in human cells. Constructs coding for the mouse and human traps were transfected in 293T cells. Cells were grown for 48 hrs post transfection in FreeStyle™ 293 medium (Invitrogen). Cell lysates were prepared from 293T cells 48 hours post-transfections. Cells were extensively washed with PBS and lysed in ice cold lysis buffer (50 mM HEPES pH7.5, 150 mM NaCL, 1.5 mM MgCl2, 1% Triton X-100 and 10% glycerol) supplemented with standard amounts of protease inhibitors (AEBSF,TPCK,TLCK, aprotinin, leupeptin, pepstatin and E64, Sigma). Cell lysates were cleared by micro centrifugation (12000 g, 20 minutes). Lysates concentrations were determined by standard micro BCA (Sigma). Equal amounts of protein were loaded on 5-20% PAGE-SDS gradient gels and transfered to PVDF (Amersham). Cleared conditioned media from transfected cells were incubated with either Protein A sepharose (Pharmacia) or Talon resin (Clontech) for FC or 6×His tag. Resins were washed with PBS and diluted in 2×PAGE-SDS sample buffer prior to gel separation and transfer. The antibody used in immunoblottings were the anti-human Neuropilin-1 (Cell signaling), the mouse monoclonal anti-6×-HIS (In Vitrogen) and the reporter HRP linked anti-human, mouse and rabbit IgG (BioRAD). All antibodies were used at a 1/2000 dilution. Chemiluminescent signal was captured using a Fuji imaging system after incubation of membranes with ECL (Amersham).

Traps expression and purification. 293-T cells were transfected with plasmids encoding the various traps by either the Polyethylamine (PEI) or the calcium phosphate precipitation standard transfections methods. The next day cells were washed twice with serum free media and fed with serum free complete media (Free style 293 media, InVitrogen). Conditioned medium were collected after 60-72 hrs of growth in serum free media and cleared from cellular debris by swing bucket centrifugation (2000 RPM, 20 minutes). FC traps were purified from conditioned media of transfected 293T cells by passage on Protein A or G sepharose (Pharmacia) followed by extensive washes with PBS and elutions with 0.1 M glycine pH 3.0. Elution fractions were neutralised immediately by the addition of 1/10 volume 1 M Tris pH 8 and 1/10 volume of 10×PBS pH 7.4. Soluble 6×HIS tagged traps were purified from conditioned media of transfected 293T cell by passage on Talon agarose (Clontech) followed by extensive washes with PBS and stepwise imidazole elutions (Range 10-150 uM typically). Samples of purification fractions of traps were analysed on 5-15% or 5-20% gradient PAGE-SDS gels. Gel were stained using the Safely Blue staining kit (InVitrogen).

Sterile formulation of purified traps for in vivo injections. Purifications elution fractions from 40 ml of conditioned media were pooled and diluted to a total volume of 10 ml in PBS. Diluted trap proteins were sterilized by filtration through a 0.2 uM low protein binding filter (Progene). Protein solutions were concentrated and buffer exchanged with PBS on sterile PES concentration devices (Pierce, nominal MWCO 30 KD). Sterile concentrated Traps samples (˜30-50 ul) were analysed and stained on PAGE-SDS as described above.

### EXAMPLE 9

### Affnity of Traps for SEMA3A

Production of AP-VEGF₁₆₅, the coding sequence of the human VEGF165 variant 1 (NM_001025366) was sub-cloned in the pAPtag5 vector (GenHunter), in-frame with an Alkaline Phosphatase domain (AP-VEGF165). HEK293T cells were transfected with the AP-VEGF165 construct using a polyethylenimine (PEI) transfection method. Following the overnight transfection step, cells were cultured for an additional 60 hr in serum free media (In vitrogen). The cell media were collected and concentrated on a PES device (Pierce). The concentrated AP-VEGF165 ligand was analysed on PAGE-SDS and quantified using SimplyBlue safe stain (Life technologies).

Sema 3A and AP-VEGF₁₆₅ binding assays. Saturation curves for the determinations of KD of binding to SEMA 3A or VEGF165 were obtained as follow. Wells of high protein binding 96 well plates (Maxisorp, Nunc) were coated with purified traps diluted in PBS and blocked afterward with binding buffer (PBS containing 2% casein and 0.05% Tween 20). The SEMA3A-FC (R&D systems) or AP-VEGF165 ligands were diluted in binding buffer over an extensive range of concentrations and added to wells. Following an overnight incubation, wells were washed with PBS containing 0.05% tween. Bound SEMA3a-FC was detected using an HRP-linked anti-Human IgG (Biorad) and ECL substrate (Pierce). Alternatively, bound AP-VEGF165 was detected using CPD star substrate (Roche). The Chemiluminescent signal was acquired on a TECAN reader. Dissociation constant (KD) were determined by non-linear curve fitting using the Graph Pad prism software.

The relative affinity of traps of the present invention to SEMA3A and VEGF has been assessed. Traps were prepared as described in Example 8. Schematic representation of traps tested (without HIS or FC tags) is also provided in FIG. 18.

TABLE 6
Dissociation constant of SEMA3A and VEGF for various traps

| | SEMA 3A-FC | VEGF165 binding |
|---|---|---|
| NRP1 Trap | binding (nM) | (nM) |
| G | 0.8 | 6.75 |
| O | 1.05 | N.D. |
| M | 0.95 | 20.13 |
| N | >1000 | >250 |
| R | 6.15 | N.D. |
| W | 1.14 | 20.73 |
| Y | >750 | N.D. |
| Z | 4.44 | 66.96 |
| AB | N.D. | 29.51 |
| AC | 4 | No binding |
| Q | No binding | N.D. |
| P | No binding | N.D. |
| X | No binding | N.D. |
| S | N.D. | 24.6 |
| AD | No binding | No binding |
| AE | No binding | No binding |
| AF | No binding | N.D. |
| AJ | 2.4 | N.D. |
| Ak | 4.4 | N.D. |

The soluble NRP1 traps tested generally bind more efficiently to SEMA3A than VEGF. Such preference for SEMA3A was found surprising since SEMA3A and VEGF are considered to normally have the same general affinity for NRP1. Applicants have also surprisingly found that introduction of a mutation at position 297 (Y297A) in NRP1 not only inhibits binding to VEGF but also to NRP1. Such mutation was previously though to be associated with Increased affinity for SEMA3A may be advantageous in conditions where SEMA3A inhibition is preferred over inhibition of VEGF. As inhibition of VEGF using VEGF inhibitors such as bevacizumab has been suggested to induce cellular senescence in colorectal cancer cells in vitro and in vivo (Hasan et al., 2011Int. J. Cancer 1;129(9) :2115-2123), the use of NRP1 traps having a reduced affinity for VEGF may be preferred in the context of senescence associated diseases and conditions. Furthermore, NRP1 traps preferably interacting with Sema3a over VEGF are expected to show reduced side effects associated with inhibition of VEGF cell signaling.

### EXAMPLE 10

### Attenuation of Cellular Senescence by NRP1 Traps

Mice subjected to OIR were intravitreally injected with NRP1 traps G or M or with vehicle at P12 and retinas were monitored for cellular senescence. As shown in FIG. 20, quantification of SA-β-gal staining of P17 OIR flatmount retinas reveals a significant attenuation of cellular senescence when mice receive a single injection of Trap M or Trap G, with Trap M being more effective in inhibiting cellular senescence. Interestingly, Trap M, has a kd for SEMA3A which is about 20× greater than for VEGF, while the preference for SEMA3A for trap G is much less important (see Table 6).

### EXAMPLE 11

### Experimental Procedures

Animals. All studies were performed according to the Association for Research in Vision and Ophthalmology (ARVO) Statement for the Use of Animals in Ophthalmic and Vision Research and were approved by the Animal Care Committee of the University of Montreal in agreement with the guidelines established by the Canadian Council on Animal Care. C57BI/6 wild-type (WT) were purchased from The Jackson Laboratory. LyzM-Cre (Lyz2^{tm1(cre)Iflo/J}; no. 004781) were purchased from The Jackson Laboratory. (C57BI/6 WT, LysM-Cre, and LysM-Cre/ROSA26EYFP^{fl/f}, we generated mice with EYFP-expressing cells of myeloid lineage (71).

O2-induced retinopathy. Mouse pups from different strains (C57BI/6 WT, LyzM-Cre, LysM-Cre/IRE1^{fl/fl}, LysM-Cre/IRE1⁺⁺, IRE1^{fl/fl} and LysM-Cre/ROSA26EYFP^{fl/fl}), and their fostering mothers (CD1, Charles River) were exposed to 75% O₂ from postnatal day 7 (P7) to day 12 and returned to room air. This model serves as a proxy to human ocular neovascular diseases such as ROP and diabetic retinopathy characterized by a late phase of destructive pathological angiogenesis (72, 73). Upon return to room air, hypoxia-driven neovascularization (NV) develops from P14 onwards (27). We enucleated eyes at different time points and dissected the retinas for mRNA, protein assays or flatmounting.

RNA-Seq samples preparation and sequencing. Total RNA was isolated from retinas using the RNeasy Mini Kit (QIAGEN). The mRNA was then purified from 1 μg of total RNA using the Dynabeads® mRNA DIRECT™ Micro Kit (Thermo Fisher SCIENTIFIC). Whole transcriptome libraries were prepared using the Ion Total RNA-seq Kit v2. The yield and size distribution of the amplified libraries were assessed with an Agilent Bioanalyzer using a DNA 1000 Kit. Sequencing was performed on an Ion Chef™ Instrument (Ion Torrent™, Thermo Fisher SCIENTIFIC).

cDNA Library Construction and Sequencing. Analysis was performed using the Torrent Suite software v4.4 (Thermo Fisher) and the whole Transcriptome Analysis Plugin v 4.2-r7 (Thermo Fisher). The whole Transcriptome Analysis Plugin aligns reads on mouse reference genome (mm10) using Tophat2 then unmapped reads are aligned using Bowtie2 and merged together. FPKM are calculated using Cufflinks.

Gene Set enrichment Analysis (GSEA). Gene set enrichment analysis was conducted using GSEA v2.2.1 software provided by Broad Institute of MIT and Harvard University. We used GSEA to validate correlation between molecular signatures in phenotype of interest. Enrichment analysis was conducted with log2-normalized Fragment Per Kilobase of transcript per Million (FPKM) data generated by the ToPhat/Cuffdiff command pipeline: FPKM values were converted as ratios (FPKM x/[FPKM Normoxia] mean), then log2 normalized (log2[ratio]) and median centered (log2 ratio−[log2 ratio Normoxia] mean).

Default parameters were changed as follow: Gene sets of interest were found in a catalog of functional annotated gene sets from Molecular signature database (MSigDB); Phenotype was permutated 1000 times; Phenotype label was defined as ‘OIR’ vs ‘Normoxia’; gene sets smaller than 15 and larger than 500 were excluded from the analysis; statistic used to score hits was defined as ‘weighted p2’, and the class separation metric used was ‘t Test’.

Semi-quantitative and Real-time PCR analysis. We isolated RNA using the GenElute™ Mammalian Total RNA Miniprep Kit (Sigma) and performed a digestion with DNase I to prevent amplification of genomic DNA. We reversed transcribed the RNA using M-MLV reverse transcriptase and analyzed gene expression using SybrGreen™ in an ABI Biosystems Real-Time PCR machine. β-actin was used as a reference gene. Primers sequences are displayed in Table 7. We investigated the splicing of XBP-1 by incubating the XBP-1 semi-quantitative PCR product with 0.4 U/μL of PstI enzyme for 5 hrs at 37° C. followed by separation on 2.5% agarose gel.

TABLE 7
Primers.

| | | SEQ | | SEQ |
|---|---|---|---|---|
| | | ID | | ID |
| Target | Forward primer | NO: | Reverse primer | NO: |
| β-actin | GACGGCCAGGTCATCACTATTG | 1 | CCACAGGATTCCATACCCAAGA | 17 |
| II1β | CTGGTACATCAGCACCTCACA | 2 | GAGCTCCTTAACATGCCCTG | 18 |
| II6 | CTCTGGGAAATCGTGGAAATG | 3 | AAGTGCATCATCGTTGTTCATACA | 19 |
| Ire1α | CCGAACGTGATCCGCTACTTCT | 4 | CGCAAAGTCCTTCTGCTCCACA | 20 |
| Cdkn2a | GGCCAATCCCAAGAGCAGAG | 5 | GCCACATGCTAGACACGCTA | 21 |
| Cdkn1a | CTCCACTGCTGCTTCCTGAG | 6 | TGCTGAGCTCATGCCCTTTG | 22 |
| TP53 | CCGTGTTGGTTCATCCCTGTA | 7 | TTTTGGATTTTTAAGACAGAGTCTTTGTA | 23 |
| Pai1 | TGACGTCGTGGAACTGC | 8 | GAAAGACTTGTGAAGTCGGC | 24 |
| SEMA3A | GGGACTTCGCTATCTTCAGAAC | 9 | GGCGTGCTTTTAGGAATGTTG | 25 |
| Tgf-β | GGACTCTCCACCTGCAAGAC | 10 | CATAGATGGCGTTGTTGCGG | 26 |
| XPB1-s | CTGAGTCCGAATCAGGTCCAG | 11 | GTCCATGGGAAGATGTTCTGG | 27 |
| Tnf-α | CGCGACGTGGAACTGGCAGAA | 12 | CTTGGTGGTTTGCTACGACGTGGG | 28 |
| Vegfa | GCCCTGAGTCAAGAGGACAG | 13 | CTCCTAGGCCCCTCAGAAGT | 29 |
| Vegfc | CAAGGCTTTTGAAGGCAAAG | 14 | AGAAGGTGTTTGTGGCTGCT | 30 |
| Vegfr-1 | GTCACAGATGTGCCGAATGG | 15 | TGAGCGTGATCAGCTCCAGG | 31 |
| Vegfr-2 | GGCGGTGGTGACAGTATCTT | 16 | GTCACTGACAGAGGCGATGA | 32 |

Flow Cytometry Analysis. Human retinal microvascular endothelial cells (HRMEC) cell cycle analysis (PI biolegend) were performed according to the manufacturer's instructions and as previously reported (43). Briefly, HRMEC (1×10⁶) were seeded in 6-well plates and incubated for 7days with SEMA3A, 100 and 500 ng/ml. The samples were analyzed by flow cytometry. FACS was performed on a LSRII (BD Biosciences) device and data were analysed using FlowJo software (version 7.6.5).

Electric Cell-substrate Impedance Sensing (ECIS) Proliferation assay. Real-time analysis of trans-endothelial electric resistance was performed by plating 5000 HRMECs/ml were seeded onto 8W10E+ standard 8-well arrays (Applied BioPhysics, NY). Cells were allowed to grow leading to a capacitance of less than 10 nF. Cells were starved for 5 Hours with endothelial basal media (EBM-2, Lonza) and then treated with 100 ng/ml SEMA3A or vehicle (EBM-2) for 120 h and impedance was measured using an ECIS Zθ impedance instrument (Applied BioPhysics, NY). Measurements were taken for 120 h post treatment.

Human samples. We obtained approval of human clinical protocol and informed consent form by Maisonneuve-Rosemont Hospital (HMR) ethics committee (Ref. CER: 10059) and recruitment of patients for local core vitreal biopsy sampling from patients afflicted with proliferative retinopathies. The entire procedure was performed as an outpatient procedure in the minor procedure room within the ambulatory clinic from the Department of Ophthalmology at Maisonneuve-Rosemont Hospital. All instruments were opened and handled in a sterile manner. The study conforms to the tenets of the Helsinki declaration.

Vitrectomy. All patients previously diagnosed with PDR were followed and operated by a single vitreoretinal surgeon (FAR). Control patients were undergoing surgical treatment for non-vascular pathology (ERM or MH) by the same surgeon. In an operating room setting, patients underwent surgery under local retro/peribulbar anesthesia. A 5% povidone-iodine solution was used to clean the periocular skin and topical instillation into the eye and within the cul-de-sac was left in place for 5 minutes. Three-port 25-gauge transconjunctival pars plana vitrectomy was performed through 25-gauge valved cannulas (Alcon). Under microscope visualization using a wide-angle viewing system (Resight, Zeiss), undiluted vitreous was collected with a 25-gauge vitrector. After vitreous biopsy, the infusion line was opened and vitrectomy and membrane peeling was performed in the usual fashion to treat diabetic vitreous hemorrhage and tractional retinal detachment. This was followed by panretinal endolaser photocoagulation, fluid-air exchange, and intravitreal anti-VEGF injection.

Quantification of Cytokines by Multiplex. Vitreous samples were frozen on dry ice and immediately after biopsy were stored at −80°. Vitreous samples were centrifuged at 15000×g for 5 minutes at 4° C. prior to analysis. Pai1, VEGF, IL-6, IL-8. A multiplex panel (Cancer Panel 1 from Bio-rad) used according to the manufacturer's protocol. The Luminex assay was analyzed using a Bio-Plex 200 array reader (Bio-rad). A quantitative determination of the respective analytes was achieved by comparing the raw data obtained from the patient samples with a standard curve. A total of 4 cytokines (Croa, Grob and IL-1β) had to be excluded because of detection I limit.

Immunofluorescence (IF). To localize protein expression, eyes were enucleated from mice and fixed in 4% paraformaldehyde for 4 h at RT and incubated in 30% sucrose overnight and then frozen in OCT compound. We then embedded the whole eye in optimal cutting temperature compound at −20° C. and performed 12 um sections. We carried out IF experiments and visualized sections with an epifluorescent microscope (Zeiss Axiolmager) or confocal microscope (Olympus confocal FV1000).

For visualization of pan-retinal vasculature, dissected retinas were flatmounted and incubated overnight with Rhodamine labeled Griffonia (Bandeiraea) Simplicifolia Lectin I (Vector Laboratories, Inc.) in 1 mM CaCl₂ in PBS for retinal vasculature. The extent of avascular area or neovascularization area at P17 using ImageJ and the SWIFT-NV method (74).

For Protein localization, flatmounted retinas were incubated with different antibodies as indicated. For in vitro IF, cultured cells were plated on 0.1% gelatin-coated coverslips and serum-starved overnight and stimulated for 7 days with SEMA3A (100 ng/ml). Cells were washed briefly with cold PBS and fixed for 20 minutes in PBS containing 3.5% paraformaldehyde. Cells were rinsed with PBS and permeabilized with 0.3% Triton in PBS for 5 minutes. Fixed cells were blocked with 1% BSA and then incubated for 1 hour with primary antibodies in 0.1% BSA in PBS. Bound primary antibodies were visualized after 1 hour of incubation using Alexa Fluor secondary antibody. Coverslips were mounted using Fluoromount (Sigma-Aldrich) and analyzed by confocal microscope (Olympus confocal FV1000). Samples were viewed with a ×63/1.4 NA oil or ×30 objective. Images were assembled using Photoshop CS4 (Adobe Systems). For all antibodies used for immunohistochemistry, see Table 8.

TABLE 8
Antibodies

| Target | Clone | Company | Catalogue no. | Application | Dilution/WB | Dilution/IHC |
|---|---|---|---|---|---|---|
| Phospho-NF-kB | p65, Ser536 | Cell Signaling Technology | 3031 | WB | 1/250 | |
| β-actin | | MEDIMABS | MM-0164-P | WB | 1/2000 | |
| P-IRE1α | S724 | Santa Cruz Biotechnology | ab48187 | WB | 1/500 | 1/100 |
| IRE1α (tot) | 14C10 | Cell Signaling Technology | 3294 | WB | 1/250 | |
| NF-kappaB | L8F6 | Cell Signaling Technology | 6956 | WB | 1/250 | 1/100 |
| P16 | F-12 | Santa Cruz Biotechnology | sc1661 | WB | 1/500 | 1/100 |
| P21 | C-19 Sc-397 | Santa Cruz Biotechnology | L1913 | WB | 1/500 | |
| P53 | FL-393 sc-6243 | Santa Cruz Biotechnology | B2013 | WB | 1/500 | |
| SEMA 3A | ab23393 | Abcam | GR26629-13 | WB | 1/500 | |
| Pai I | H-135 Sc-8979 | Santa Cruz Biotechnology | E2214 | WB | 1/500 | 1/100 |
| XBP1 | M-186 Sc-7160 | Santa Cruz Biotechnology | G2415 | WB | 1/500 | |
| H2AX | 15A3 | Abcam | ab62623 | WB | 1/1000 | 1/100 |
| (8Hydroxyguanosine) | | | | | | |
| Cre-recombinase | 2D8 | millipore | MAB3120 | WB | 1/500 | 1/100 |
| IBA1 | | Wako | | IHC | | 1/200 |
| Cleaved caspase-3 | (Asp175) | Cell Signaling | | WB | 1/1000 | |
| Brn3a | C-20 | Santa Cruz Biotechnology | Sc-31984 | IHC | | 1/200 |
| α-SMA | Gr43049-4 | Abcam | ab7817 | IHC | | 1/200 |
| NG2 | | Abcam | ab50009 | IHC | | 1/100 |

Senescence-associated β-galactosidase (SA-β-gal) assay. Senescence-associated β-galactosidase assays were carried out as described previously (57, 75)

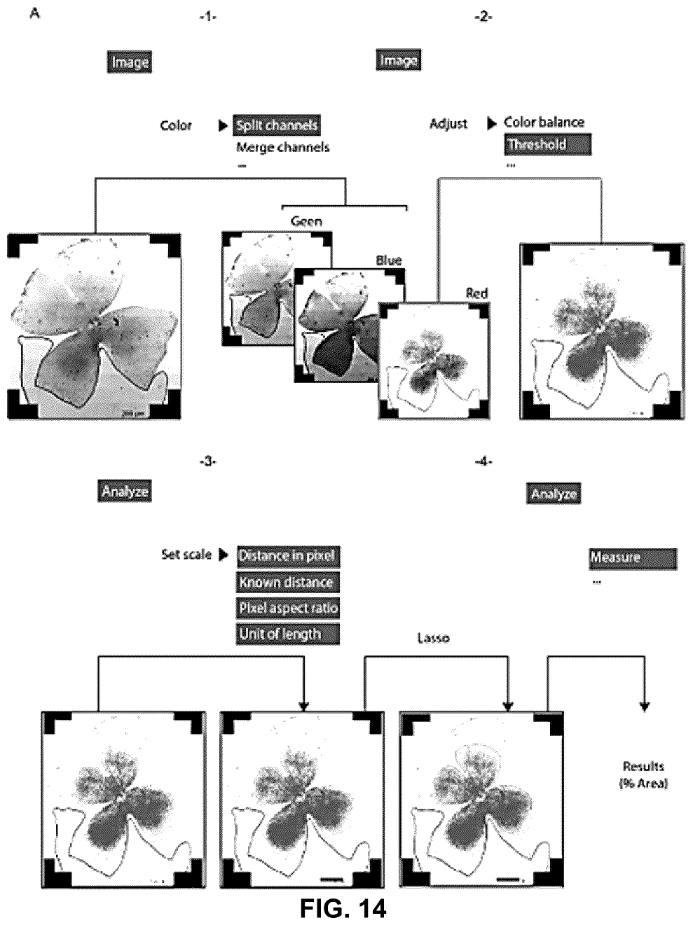

Quantification of SA-β-gal in vivo. Senescence-associated β-galactosidase staining in flatmount retinas or sagittal eye sections were analyzed using Image J software as described in FIG. 14.

Lentivirus production. Lentiviral vectors (HIV-1 derived) were prepared by transfecting HEK293T cells HEK293T cells (Invitrogen) as previously described by us and others (35, 44, 76) with a vector plasmid containing Cre, green fluorescent protein GFP or the small hairpin RNAs (Sh_RNAs) against SEMA3A, IRE1α or GFP (see Table 9 below) together with the third-generation packaging plasmids pV-SVG, pMDL, and pREV (Open Biosystems). Approximately 10⁷ cells were seeded and transfected with the above plasmids in DMEM complete medium (Invitrogen) and incubated for 30 hours. Subsequently, supernatant was replaced with fresh complete DMEM medium and incubated for an additional 30 hours. Secreted virus was collected and ultracentrifuged at 50 000 g, resuspended in PBS, aliquoted, and stored at −80° C.

Intravitreal injections. P2, P10 or P12 C57BL/6 pups were anesthetized with 3.0% isoflurane and injected in the vitreous chamber with 0.5 μL of lentivirus (see “Lentivirus production”), recombinant SEMA3A (100 ng/μL), metformin (10 μg/μL) or Aflibercept (10 μg/uL) using a 10-μL Hamilton syringe fitted with a 50-gauge glass capillary tip. Approximately 254±11.0 ng/μL of lentivirus Sh_GFP and 323.3±15.3 ng/μL containing Sh_Sema3a, Lv.Cre (15.0 ng/ml), Lv.GFP (15.0 ng/ml) was injected. Virus titers were assessed with the p24 ELISA kit (ZeptoMetrix). The titers of the lentiviruses used were (in ng p24) LV.Sh_RNA IRE1α (8.52 ng/mL), and LV.Sh_RNA.GFP (8.47 ng/ml).

TABLE 9
shRNAs

| | | Antisense target | |
|---|---|---|---|
| | | sequence (in | |
| | | Mature antisense | SEQ |
| | | sequence T | ID |
| Target | Ref. | changed to U) | NO: |
| hSEMA3A | TR0N0000058138 | AAATCCTTGATATTAACCAGG | 33 |
| hSEMA3A | TR0N0000058139 | TTTCCCGTAAATATCACACCG | 34 |
| hSEMA3A | TR0N0000058142 | TTGAAACTACTTTAAGAACGG | 35 |
| hSEMA3A | TR0N0000058140 | AAATTAGCACATTCTTTCAGG | 36 |
| mSEMA3A | TR0N0000067328 | AAATTGCCAATATACCAAGGC | 37 |
| mSEMA3A | TR0N0000067331 | AATGAGCTGCATGAAGTCTCG | 38 |
| mSEMA3A | TR0N0000067330 | AAATTGGCACATTCTTTCAGG | 39 |
| mSEMA3A | TR0N0000067329 | TTCATTAGGAATACATCCTGC | 40 |
| mSEMA3A | TR0N0000067332 | TTATTTATAGGAAACACTGGG | 41 |
| IRE1α | | AACGCCACCCATCCAACCA | 42 |
| shGFP | | GCAAGCTGACCCTGAAGTTCAT | 43 |

Preparation of conditioned media (CM). Human retinal microvascular endothelial cells (HRMECs), retinal neuron 661 W photoreceptor cells and Mouse macrophages (J774A.1 cell line) were incubated for 7 days with recombinant SEMA3A(100 ng/μL), H₂O₂ (150 μM for 2 h) 48 h after transfection or not as indicated in each experiment. Supernatants were centrifuged and filtered and then frozen for subsequent use. For Western Blot on CM was concentrated using ultra centrifugal amicon filter unit from Millipore.

Western blotting. We enucleated eyes at varying time points and rapidly dissected and homogenized retinas for assessment of retinal protein levels. Protein concentration from retinal homogenate and cell lysates were assessed by BCA assay (Sigma), and then 30 μg of protein analyzed for each condition by standard SDS-PAGE technique. Antibodies used for Western-blotting are listed in Table 8 above.

Statistical analyses. We used Student's T-test and ANOVA, where appropriate. A P<0.005 and P<0.05, respectively was considered statistically different using Prism, version 5 software (GraphPad).

Recombinant proteins used. Recombinant human Semaphorin 3A (from murine myeloma cell line, NS0) (R&D Systems) concentration used in vitro 100 and 500 ng/ml and 100 ng/ml in vivo.

Materials. Metformin, assay (RIPA) buffer, protease inhibitor cocktail, and phosphatase inhibitors were purchased from Sigma Chemicals. Aflibercept (Eylea™) was purchased from Bayer. 4μ8c inhibitor was from Torcis (Biosciences).

Plasmids and generation of Stable Cell Lines and Transfections. We stably transfected 661 W cells and HRMECs (Open Biosystems) cells with 500 ng of Sh_RNA plasmids targeting, Sema3a, IRE1α respectively and an unrelated sh_RNA (sh_GFP) for 16 hr at 37_C using Lipofectamine™ 2000 following the manufacturer's directions. We generated stable cell lines by selecting with 2 mg of puromycin over 2 weeks. Expression plasmids for GFP, IRE1α WT, dominant-negative mutant of IRE1α, the RNase dead mutant K907A in J774 cells using Lipofectamine™ 2000. Plasmids for IRE1α were obtained from Addgene (Fumihiko Urano: plasmids #20744 and #20745).

### EXAMPLE 12

### Experimental Procedures for Examples 13-15

Mice. All studies were performed according to the guidelines of the Canadian Council on Animal Care and were approved by the Animal Care Committee of the University of Montreal. C57BI/6 wildtype mice, LysM-Cre mice (B6.129P2-Lyz2^{tm1(cre)/fo}/J ; no.004781), and Neuropilin-1 floxed mice (B6.129(SJL)-NRP1^{tm2Ddg}/J; no. 005247), were purchased from The Jackson Laboratory and bred in house. Diets: HFD: 60% fat calories, BioServ F3282; control feed: 2018 Teklad Global 18% protein rodent diet.

Fluorescence-activated Cell Sorting (FACS) of adipose tissue macrophages Retroperitoneal fat pads were collected, weighted and homogenized in DMEM F12 medium then incubated with 1 mg/ml of collagenase D (Sigma) at 37° C. for 45 minutes. EDTA was then added at a concentration of 10 mM and the mix was incubated for an extra 5 minutes. Homogenates were then filtered with a 70-μm cell strainer and centrifuged. Pellets were resuspended and incubated in lysis buffer (10 mM KCHO₃; 150 mM; NH₄Cl; 0.1 mM EDTA) for 5 minutes at room temperature and centrifuged. Pellets were resuspended in 1×PBS and filtered with a 100-μm cell strainer. Cell suspensions were incubated with Zombie Aqua Fixable Viability Kit (BioLegend) for 15 minutes at room temperature. Cells were then incubated with LEAF purified anti-mouse CD16/32 (Biolegend) for 15 minutes at room temperature to block Fc receptors. Cells were then incubated for 25 minutes at 4° C. with the following antibodies: Brilliant Violet 785 anti-mouse CD45.2 (BioLegend), Brilliant Violet 711 anti-mouse/human CD11b (BioLegend), APC/CY7 anti-mouse Ly-6G (BioLegend), Pe/Cy7 anti-mouse F4/80 (BioLegend), PE antimouse CD11c (BioLegend), FITC anti-mouse Ly-6C (BioLegend) and APC anti-mouse CD304 (Neuropilin-1) or APC Rat IgG2a, κ Isotype Ctrl (BioLegend). For analysis of CD206 expression, permeabilisation and fixation of the cells was done using the Cytofix/Cytoperm kit (BD Bioscience) at 4° C. for 20 minutes. Cells were then incubated with Rat serum (Cedarlane) for 25 minutes at 4° C. in order to block intracellular receptors. Cells were finally stained with Brilliant Violet 421 anti-mouse CD206 (MMR) (BioLegend) for 25 minutes at 4° C. FACS was performed on a Fortessa (BD Biosciences) device, and data were analyzed using FlowJo software (version 7.6.5).

In vivo BODIPY uptake. In vivo BODIPY intake assays were performed on LysM-Cre-NRP1^{+/+} and LysM-Cre-NRP1^{fl/fl} male mice fed with HFD for 10 weeks. Mice were starved for four hours before administrating an intraperitoneal injection of 100 μL of 30 μM BODIPY™ 500/510 C1, C12 in 1% BSA. Mice were euthanized 3 hours following BODIPY™ injection. The blood was collected by cardiac puncture, and the plasma was subsequently separated by centrifugation. Samples of heart, liver and white adipose tissue were collected and homogenized in 1×RIPA buffer (Cell Signaling). BODIPY™ fluorescence of homogenates and plasma was read with Infinite M1000 Pro reader (Tecan) at a wavelength emission of 488 nm and excitation at 525 nm and normalized to protein concentration (quantified with QuantiPro™ BCA assay kit from Sigma).

Primary macrophages culture 8-12 week old LysM-Cre-NRP1^{+/+} and LysM-Cre-NRP1^{/fl/fl} mice were anesthetized with 2% isoflurane in 2 L/min oxygen and then euthanized by cervical dislocation. Then, a small incision in abdominal skin of mouse was performed. Skin was pulled to each size of the mouse and peritoneal cavity was washed with 5 ml of PBS plus 3% FBS for 2 min. Then, the harvested cells were centrifuged for 5 min at 1000 rpm, resuspended in medium (DMEM F12 plus 10% FBS and 1% Streptomycin/Penicillin) and plated. After 1 h of culture at 37° C. under a 5% CO₂ atmosphere the medium was changed and cells were cultured for the next 24 h in the same conditions before use in BODIPY uptake, pHrodo phagocytosis assay, or Oil Red-O staining.

Quantitative RT-PCR (qPCR) analysis. RNA extraction was performed with 100-500 mg of frozen (−80° C.) RP-WAT following the Trizol Reagent Protocol (Invitrogen). Total RNA (1 μg) was reverse transcribed according to the manufacturer's instructions (iScript cDNA synthesis kit, Bio-Rad). qPCR was performed using SYBR Green (Bio-Rad) and 40 ng cDNA per reaction (7500 Real-Time PCR System, Applied Biosystem). Expression levels were normalized to the expression of b-actin. Primers (Integrated DNA Technologies) sequences are listed as follows:

TABLE 10
Sequences of primers used for qRT-PCR

| | Forward (5′→3′) | | Reverse (5′→3′) | |
|---|---|---|---|---|
| | | SEQ | Sequence | SEQ |
| | | ID | | ID |
| Genes | Sequence | NO: | | NO: |
| NRP1 | ACCCACATTTCG | 99 | TTCATAGCGGAT | 100 |
| | ATTTGGAG | | GGAAAACC | |
| SEMA3a | GCTCCTGCTCCG | 101 | TCGGCGTTGCTT | 102 |
| | TAGCCTGC | | TCGGTCCC | |
| SEMA3e | TCTGCAACCATC | 103 | ACCACAAGAGGG | 104 |
| | CA | | AAGCACAGAC | |
| TGFb | GGACTCTCCACC | 105 | CATAGATGGCGT | 106 |
| | TGCAAGAC | | TGTTGCGG | |
| VEGFa | GCCCTGAGTCAA | 107 | CTCCTAGGCCCC | 108 |
| | GAGGACAG | | TCAGAAGT | |
| VEGFb | TCTGAGCATGGA | 109 | TCTGCATTCACA | 110 |
| | ACTCATGG | | TTGGCTGT | |

ImmGen skyline dataset. Immunological Genome Project data Phase 1 (GEO accession code GSE15907) and phase 2 (GSE37448) were extracted and normalized in R by Robust Multi array Average (RMA), antiLog values were ploted.

Immunohistochemistry (IHC). RPWAT tissue was fixed in 4% PFA for 48 hours then incubated in 20% methanol for 10 minutes and rinced in PBS. 1 hour blocking in 3% BSA (Hyclone, GE)+0.3% Triton™ X-100 (Sigma) preceded overnight incubation with Rhodamine-labeled Griffonia (Bandeiraea) Simplicifolia Lectin I (Vector Laboratories Inc.), anti-rat F4/80 (Donkey IgG; eBioscience), anti-rabbit Perilipin (Donkey IgG; Abcam), anti-rat Neuropilin-1 antibody, (Donkey IgG; R&D Systems) at 4° C. Alexa-Fluor secondary antibodies were incubated for two hours at 20° C. The RPWAT was then mounted onto a microscope slide and images were taken by confocal microscope.

Macrophage BODIPY intake. Macrophages extracted from LysMCRE-NRP1^{+/+} and LysMCRE-NRP1^{fl/fl} were seeded in 48 well plates at 1×10⁵ cells/well. BODIPY 500/510 C1,C12 (Life technologies) was added at a concentration of 0.5 and 1 μg/mL, incubated at room temperature for five minutes, then put on ice. Wells were washed with cold PBS then fixed with 1% paraformaldehyde (Electron Microscopy Science). Fluorescence was read with an Infinite M1000 Pro reader (Tecan) at a wavelength emission of 488 nm and 525 nm excitation. Cells were then stained with DAPI (Life Technologies) at a concentration of 1/20 000 and fluorescence measured at 358 nm excitation, 461 nm emission.

pHrodo phagocytosis assay. Macrophages extracted from LysMCRE-NRP1^{+/+} and LysMCRE-NRP1^{fl/fl} were seeded in 96 well plates at 1×10⁵ cells/well. pHrodo® Green Zymosan Bioparticles Conjugate® (Life Technologies) was resuspended at a concentration of 0.5 mg/ml in FluoroBrite™ DMEM Media+10% FBS+1% PenStrep. 100 μL of the bioparticle resuspension was added to the cells and empty wells as a negative control. Cells were incubated 90 minutes at 37° C., and pH/phagocytosis-dependent fluorescence was detected on a TECAN plate reader at 509 nm excitation and 533 nm emission. Net phagocytosis was calculated by subtracting negative control fluorescence from that of the experimental samples.

Oil Red-O staining and quantification. Cultured adipocytes and peritoneal macrophages were washed in PBS and fixed in 10% PFA for 30 minutes and rinsed. Cells were then incubated for 60 minutes with twice filtered 0.3% Oil Red-O solution and rinsed. Pictures were taken under light microscopy at a 10× magnification for the adipocytes and 63× for the macrophages. Lipid droplet quantification was performed using the Limit of threshold method from ImageJ.

Weight gain in presence of adeno Trap M protocol. C57BI6/J mice at 6-8 weeks of age were separated in 6 groups (Regular diet+Saline, Regular diet+adeno Trap M, Regular diet+adeno GFP, High fat diet+Saline, High fat diet+adeno Trap M, High fat diet+adeno GFP). Mice were intravenously injected (tail vein) with saline, Adeno-Trap M or Adeno GFP (0.25×10¹⁰ PFU /injection). Half of these mice were fed a high fat diet and the other half a regular diet and weighed at weekly intervals.

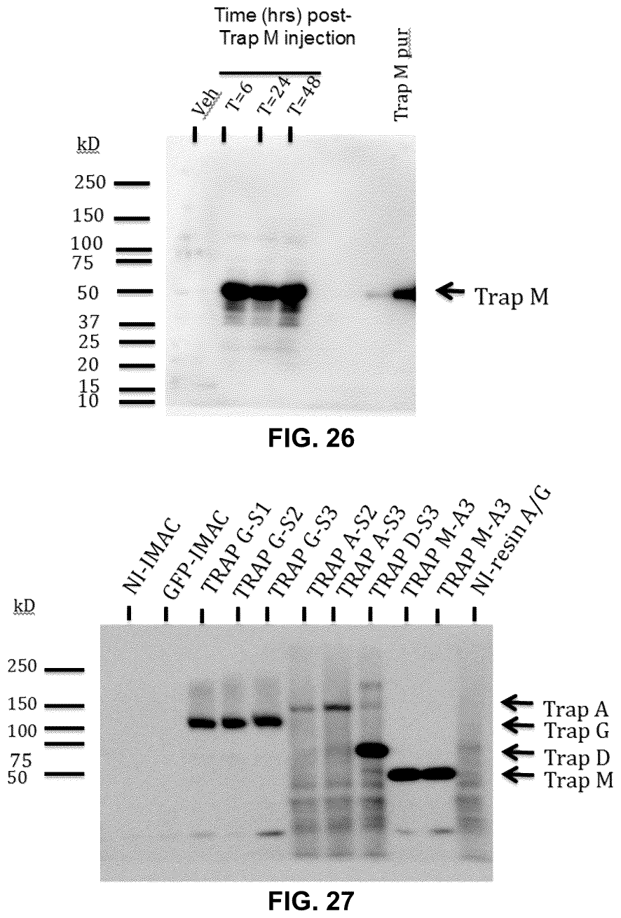

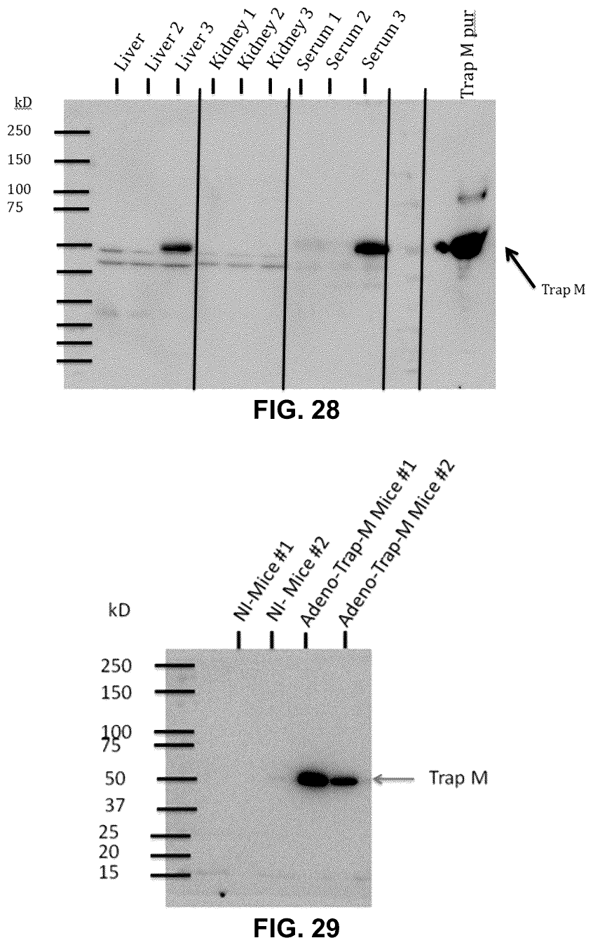

Two and eight weeks after injections, a drop of blood was taken from the tail. The presence of Trap M was assessed in the blood by immunoprecipitation using an anti-His antibody (see FIGS. 26, 28 and 29).

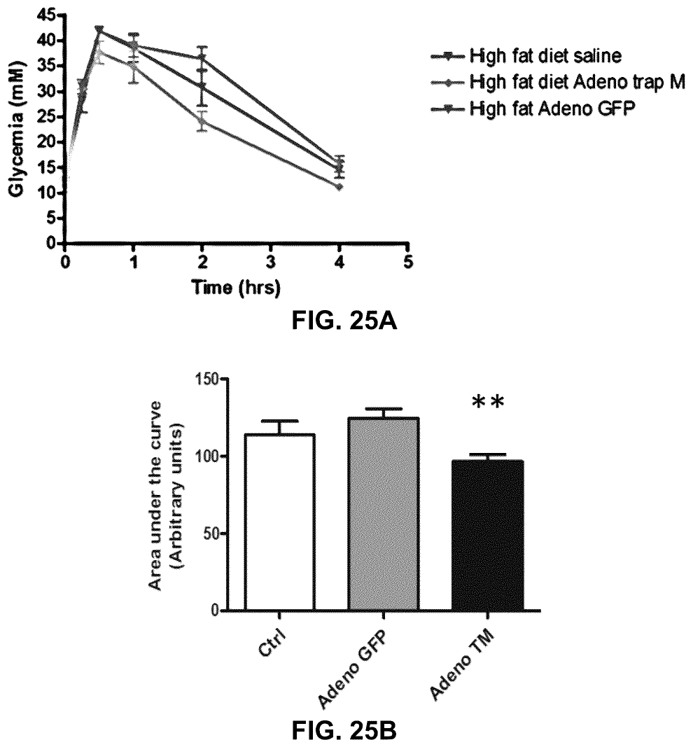

Glucose Tolerance Test (GTT). C57BI6/J mice at 6-8 weeks of age were intravenously injected with saline or Adeno-Trap M (0.25×10¹⁰ PFU /injection). Mice were fed a high fat diet right after injection. Glycemia was assessed at baseline, 15, 30, 60, 120 and 240 minutes following intraperitoneal injection of 2 g of D-glucose/kg. Measurements recorded are shown in FIG. 25 (N=5, and N.S means not significant in Two-way Anova Bonferroni posttest).

Insulin Tolerance Test (ITT). Mice were starved 5.5 hours (in the morning). Blood glucose was measured at baseline, 30, 60 and 120 minutes following intraperitoneal injection of 0.75 U/kg of insulin.

In vivo BODIPY™ uptake. In vivo BODIPY™ intake assays were performed on LysM-Cre-NRP1+/+ and LysM-Cre-NRP1fl/fl male mice fed with HFD for 10 weeks. Mice were starved for four hours before administrating an intraperitoneal injection of 100 μL of 30 μM BODIPY 500/510 C1, C12 (Life technologies) in 1% BSA (Hyclone, GE). Mice were euthanized 3 hours following BODIPY injection. The blood was collected by cardiac puncture, and the plasma was subsequently separated by centrifugation. Samples of heart, liver and white adipose tissue were collected and homogenized in 1×RIPA buffer (Cell Signaling). BODIPY fluorescence of homogenates and plasma was read with Infinite M1000 Pro reader (TECAN) at a wavelength emission of 488 nm and excitation at 525 nm and normalized to protein concentration (quantified with QuantiPro™ BCA assay kit from Sigma).

Oil Red O stain and quantification. Cultured adipocytes and peritoneal macrophages were washed in PBS and fixed in 10% PFA for 30 minutes and rinsed. Cells were then incubated for 60 minutes with twice filtered 0.3% Oil Red-O (Sigma) solution and rinsed. Pictures were taken under light microscopy at a 10× magnification for the adipocytes and 63× for the macrophages. Lipid droplet quantification was performed using the limit of threshold method from ImageJ.

Adenovirus production. Traps AD and AE were derived from Trap M and O (previously described WO2016/033699) by introduction of the VEGF165 binding mutant residue D320K using the Q5 site directed mutagenesis kit (New England Biolabs). Adenovirus trap constructs were generated by first sub-cloning the coding sequences of Traps M, G, A and D into the EcoRI-EcoRV site of pENTR1A (Life technologies) followed by LR clonase homologous recombination in the destination vector pAd/CMV/V5-DEST (Life technologies). The current set of constructs are referred to as pAdeno-Trap A, C,G or M and pAdeno-GFP. All constructs insert sequences were verified by Sanger sequencing (Genome Quebec). All junction regions generated after trap coding sequence recombination into pAD/CMV/V5-dest were sequenced as well.

Statistical analyses. Data are presented as mean±SEM. A 2-tailed Student's t test and ANOVA were used, where appropriate, to compare the different groups. P<0.05 was considered statistically different.

### EXAMPLE 13

### NRP1-Expressing Macrophages Accumulate in Adipose Tissue During Diet-Induced Obesity

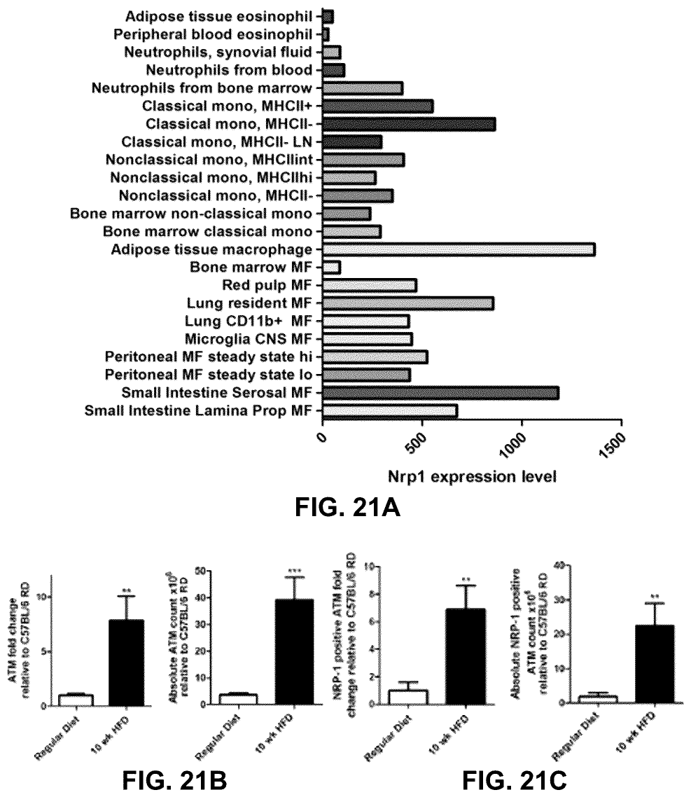

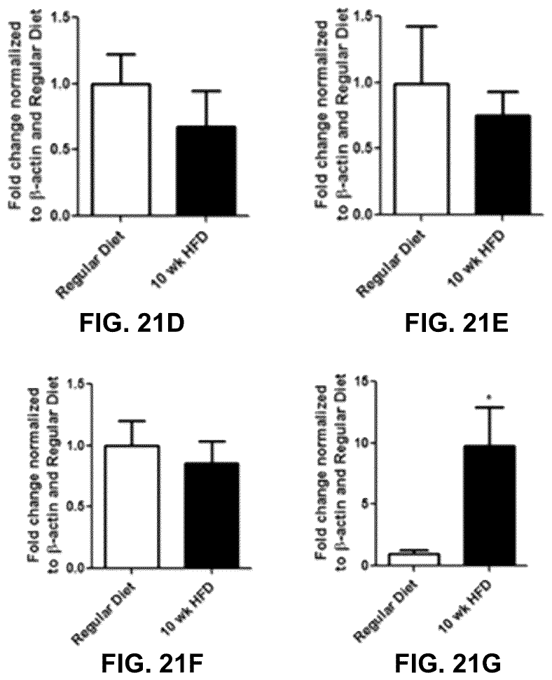

Upon Diet-induced obesity (DIO), necrotic adipocytes release Fatty acids (FA) are partially taken up by surrounding macrophages forming crown-like structures. In view of the importance of macrophages in lipid metabolism and obesity, the expression profiles of NRP1 in myeloid cells were analyzed using data from the immunological consortium ImmGen (Heng and Painter, 2008). Expression of NRP1 was most robust in adipose tissue macrophages (ATMs) compared to other steady state tissue-resident macrophages, monocytes and neutrophils (FIG. 21A). This data pointed to a potential role of NRP1+ macrophages in adipose tissue homeostasis.

Therefore C57BL/6 mice were placed on high fat diet (HFD; 60% fat calories) for 10 weeks starting at 8 weeks of life and ATM populations were investigated by Fluorescence-activated Cell Sorting (FACS). In accordance with other studies, an increased presence of ATMs was detected in adipose tissue of HFD-fed mice when compared to age matched controls on regular diet (RD; 18% fat calories) (FIG. 21B). This was paralleled by a proportionate increase in NRP1+ ATMs (FIG. 21C). Immunohistochemistry (IHC) of retroperitoneal white adipose tissue (RPWAT) from both 10 week HFD-fed mice and age matched RD mice confirmed robust expression of NRP1 on macrophages and vessels (data not shown). After 22 weeks of HFD, NRP1 localized to crown-like structures, which correspond to clusters of phagocytic macrophages surrounding dying and dead adipocytes (data not shown). Of the NRP1 ligands investigated, only Transforming Growth Factor Beta 1 (Tgfb1) rose significantly in the retroperitoneal white adipose tissue (RPWAT) of HFD-fed mice (FIG. 21G), while Semaphorin-3A (Sema3a), Vascular Endothelial Growth Factor-A (Vegfa) or -B (Vegfb) were unaffected (FIGS. 21D-F). Together, these data demonstrate robust expression of NRP1 in ATMs and suggest accretion of NRP1+ macrophages in adipose tissue during HFD-induced weight gain.

### EXAMPLE 14

### NRP1 Promotes Fatty Acid Uptake and Phagocytosis by Macrophages

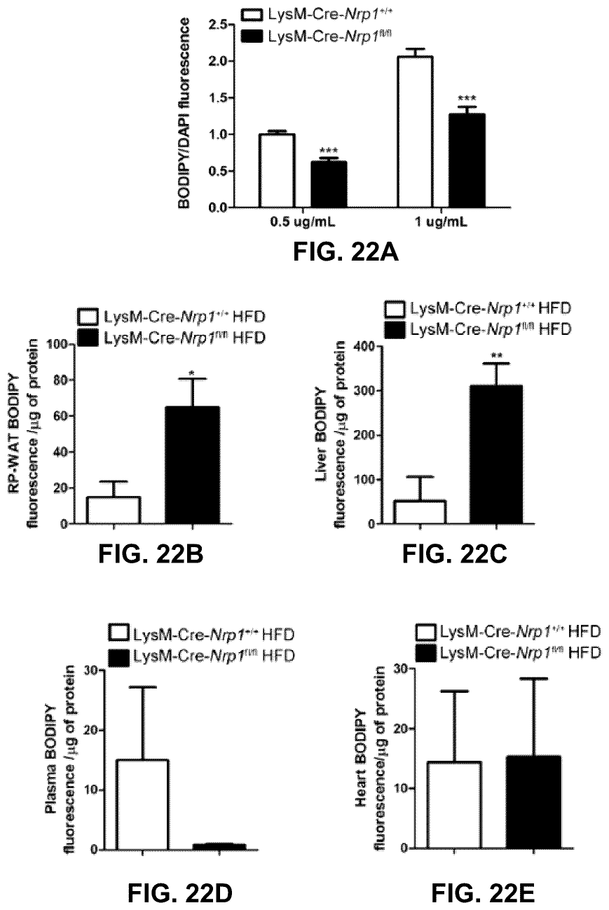

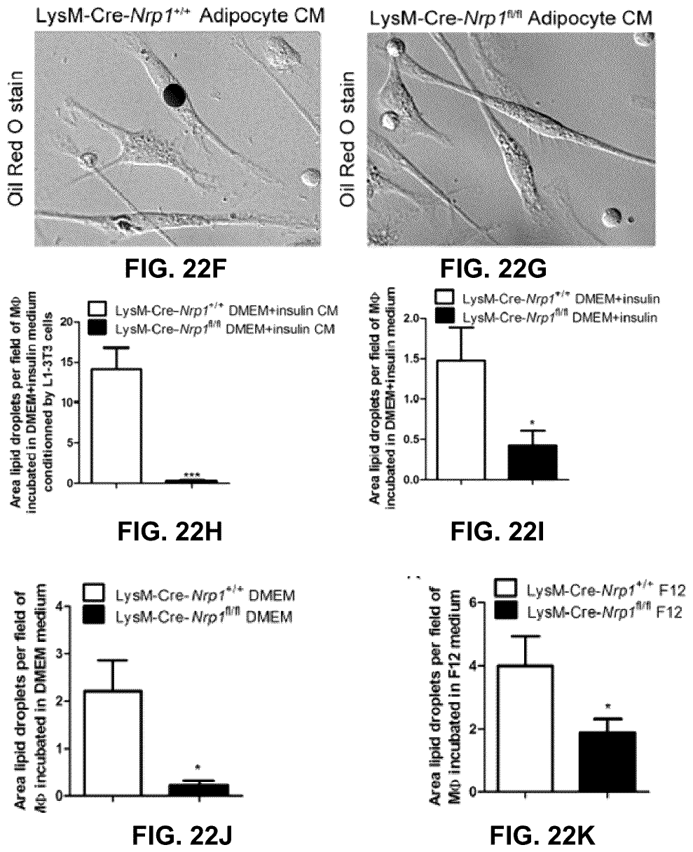

In obesity, long chain Fatty acid (FA) uptake is upregulated in adipocytes (Berk et al., 1999; Petrescu et al., 2005). To elucidate the role of NRP1+ macrophages in adipose tissue homeostasis and weight gain, a LysM-CRE-NRP1^{fl/fl} mouse line was generated with NRP1 specifically ablated in cells of myeloid lineage (Dejda et al., 2014). The uptake of a long chain FA analogue (C1-BODIPY-C12, an 18-carbon FA) was therefore measured in LysM-Cre-NRP1^{fl/fl} and control LysM-Cre-NRP1^{+/+} macrophages. NRP1-deficient macrophages took up significantly less FAs than control macrophages during acute exposure (FIG. 22A). In addition, systemic administration of C1-C12 BODIPY revealed significantly elevated levels of the tagged FAs in RPWAT and liver of LysM-Cre-NRP1^{fl/fl} mice (FIGS. 22 B, C) when compared to plasma and heart (FIGS. 22 D, E) solidifying the role of NRP1⁺ macrophages in lipid uptake.

To determine if NRP1 affected lipid sequestering in macrophages, neutral lipids within macrophages were stained with Oil Red O. Oil Red O stain was significantly reduced in LysM-Cre-NRP1^{fl/fl} macrophages incubated in adipocyte-conditioned medium (FIGS. 22F-G). Because adipocyte and macrophage media differ in glucose and insulin concentration, we assessed if the decrease in internalized lipids in NRP1-deficient macrophages also occurred in non-conditioned media, including adipocyte medium with and without insulin, as well as macrophage medium. In all conditions, NRP1 macrophages sequestered significantly fewer neutral lipids than controls (FIGS. 22 H-K).

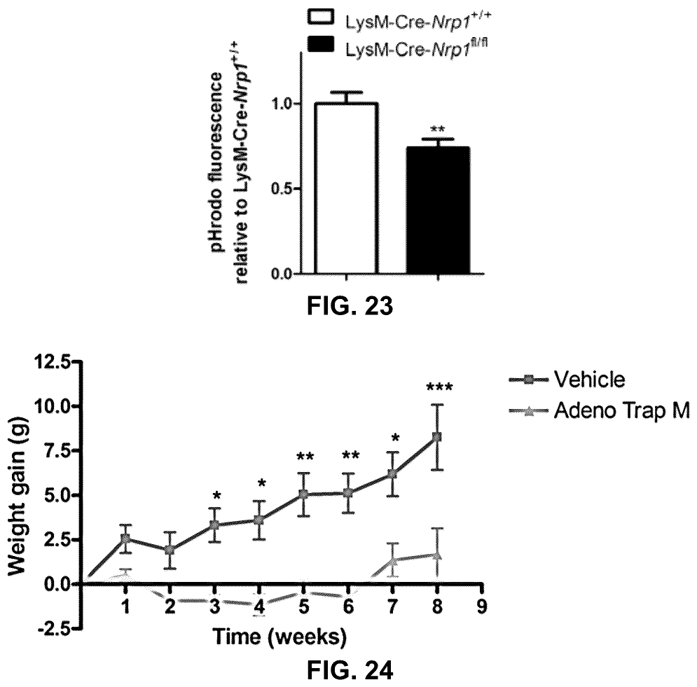

As adipocyte death increases in obese mice and humans, it lures macrophages to necrotic sites in order to phagocytose cellular debris and sequester released lipids (Cinti et al., 2005). Having observed reduced lipid uptake in NRP1 deficient macrophages, we questioned whether their phagocytic capacities were also compromised. Phagocytosis was measured with the pHrodo green zymosan bioparticles conjugate in LysM-Cre-NRP1^{fl/fl} and control macrophages, and found that macrophages lacking NRP1 had a decreased phagocytic capacity (FIG. 23).

In summary, the above results demonstrate that NRP1 deficient macrophages have impaired FA uptake and phagocytic capacity.

### EXAMPLE 15

### NRP1 Trap Reduces Weight Gain Associated with High Fat Diet

The effect of an NRP1 trap on weight gain was assessed. An adeno virus expressing a soluble NRP1 trap comprising domains a1, a2 and b1 of NRP1 (Trap M, see Table 2); Adeno GFP; or saline (control) was administered to male mice and at the same time mice were switched from a regular diet to a high fat diet (HFD, T0). Weight gain was monitored over a period of 10 weeks. Data are presented as mean±SEM. Student's unpaired t-test, *p<0.05, **p<0.01, Saline vs Adeno Trap M, Two-way Anova, Bonferroni posttest, wherein N=5.

As shown in FIG. 24, administration of NRP1 prevented weight gain in mice. The increase in weight gain observed at weeks 7 and 8 coincides with a decrease in circulating adenovirus expressing NRP1-trap. Surprisingly, prevention of weight gain was more important in male mice than in female mice (data not shown).

The effect of NRP1 traps on glucose tolerance was also assessed. Six to height (6-8) weeks old C57BI6/J mice were intravenously injected with saline, Adeno GFP or Adeno Trap M and fed a high fat diet right after injection. Glycemia was assessed at different time-points after intraperitoneal injection of 2 g of glucose/kg mice. As shown in FIG. 25B mice treated with Adeno Trap M were more tolerant to glucose than mice treated with saline or Adeno GFP.

TABLE 11
SEQ ID NOs. of sequences disclosed herein

| SEQ ID NO: | DNA/PRT | Description |
|---|---|---|
| 1-32 | DNA | Oligos listed in Table 6 |
| 33-43 | DNA | shRNAs target sequence listed in Table 9 |
| 44-46 | PRT | Human soluble Neuropilin-1 (NRP1) protein sequences |
| | | shoen in FIG. 19 and described in Table 2 |
| 47 | PRT | Consensus sequence (variant) derived from alignment of |
| | | FIG. 17 and known variants. |
| 48 | PRT | Mouse NRP1 precursor FIG. 17 |
| 49 | PRT | Rat NRP1 precursor FIG. 17 |
| 50 | PRT | Human Sema3A precursor protein shown in FIG. 16 |
| 51, 53, 55, 57, 59, | DNA | Traps AD, AE, AF, AG, AJ, AK, AR, AS, G, R, Z, AB, AC, |
| 61, 63, 65, 67, 69, | | O, Q, M, P, N, W, X, Y and S (see Table 2)-Includes signal |
| 71, 73, 75, 77, 79, | | peptide |
| 81, 83, 85, 87, 89, | | |
| 91 and 93 | | |
| 52, 54, 56, 58, 60, | PRT | Traps AD, AE, AF, AG, AJ, AK, AR, AS, G, R, Z, AB, AC, |
| 62, 64, 66, 68, 70, | | O, Q, M, P, N, W, X, Y and S (see Table 2) |
| 72, 74, 76, 78, 80, | | |
| 82, 84, 86, 88, 90, | | |
| 92 and 94 | | |
| 95 | PRT1 | Human NRP1 isoform 1, full length (cellular form) |
| 96 | PRT1 | NRP1 functional variant (Origen sequence), full length |
| | | (cellular form) |
| 97-98 | PRT1 | Exemplary peptide sequences recognized by TEV protease |
| 99-110 | DNA | qRT-PCR primers set forth in Table 10 |

The scope of the claims should not be limited by the preferred embodiments set forth in the examples, but should be given the broadest interpretation consistent with the description as a whole.

### REFERENCES

1. P. Sapieha et al., Proliferative retinopathies: angiogenesis that blinds. Int J Biochem Cell Biol 42, 5-12 (2010).
2. L. P. Aiello et al., Suppression of retinal neovascularization in vivo by inhibition of vascular endothelial growth factor (VEGF) using soluble VEGF-receptor chimeric proteins. Proc Natl Acad Sci U S A 92, 10457-10461 (1995).
3. L. Aiello, Perspectives on diabetic retinopathy. American journal of ophthalmology 136, 122-135 (2003).
4. M. E. Hartnett, J. Penn, Mechanisms and management of retinopathy of prematurity. The New England Journal of Medicine 367, 2515-2526 (2012).
5. A. Hellström, L. E. H. Smith, O. Dammann, Retinopathy of prematurity. The Lancet-British Edition 382, 1445-1457 (2013).
6. J. H. Kempen et al., The prevalence of diabetic retinopathy among adults in the United States. Arch Ophthalmol 122, 552-563 (2004).
7. D. A. Antonetti, R. Klein, T. W. Gardner, Diabetic retinopathy. N Engl J Med 366, 1227-1239 (2012).
8. H. Sakai, Y. Tani, E. Shirasawa, Y. Shirao, K. Kawasaki, Development of electroretinographic alterations in streptozotocin-induced diabetes in rats. Ophthalmic Res 27, 57-63 (1995).
9. H. A. Hancock, T. W. Kraft, Oscillatory potential analysis and ERGs of normal and diabetic rats. Invest Ophthalmol Vis Sci 45, 1002-1008 (2004).
10. K. Shinoda et al., Early electroretinographic features of streptozotocin-induced diabetic retinopathy. Clin Experiment Ophthalmol 35, 847-854 (2007).
11. A. J. Barber et al., The Ins2Akita mouse as a model of early retinal complications in diabetes. Invest Ophthalmol Vis Sci 46, 2210-2218 (2005).
12. A. J. Barber et al., Neural apoptosis in the retina during experimental and human diabetes. Early onset and effect of insulin. J Clin Invest 102, 783-791 (1998).
13. M. J. Gastinger, A. R. Kunselman, E. E. Conboy, S. K. Bronson, A. J. Barber, Dendrite remodeling and other abnormalities in the retinal ganglion cells of Ins2 Akita diabetic mice. Invest Ophthalmol Vis Sci 49, 2635-2642 (2008).
14. V. Asnaghi, C. Gerhardinger, T. Hoehn, A. Adeboje, M. Lorenzi, A role for the polyol pathway in the early neuroretinal apoptosis and glial changes induced by diabetes in the rat. Diabetes 52, 506-511 (2003).
15. T. S. Kern, A. J. Barber, Retinal ganglion cells in diabetes. J Physiol 586, 4401-4408 (2008).
16. P. Sapieha et al., Omega-3 polyunsaturated fatty acids preserve retinal function in type 2 diabetic mice. Nutr Diabetes 2, e36 (2012).
17. D. Munoz-Espin et al., Programmed cell senescence during mammalian embryonic development. Cell 155, 1104-1118 (2013).
18. M. Storer et al., Senescence is a developmental mechanism that contributes to embryonic growth and patterning. Cell 155, 1119-1130 (2013).
19. D. Munoz-Espin, M. Serrano, Cellular senescence: from physiology to pathology. Nat Rev Mol Cell Biol 15, 482-496 (2014).
20. M. Demaria et al., An essential role for senescent cells in optimal wound healing through secretion of PDGF-AA. Dev Cell 31, 722-733 (2014).
21. D. J. Baker et al., Naturally occurring p16(Ink4a)-positive cells shorten healthy lifespan. Nature 530, 184-189 (2016).
22. D. J. Baker et al., Clearance of p16Ink4a-positive senescent cells delays ageing-associated disorders. Nature 479, 232-236 (2011).
23. B. G. Childs, M. Durik, D. J. Baker, J. M. van Deursen, Cellular senescence in aging and age-related disease: from mechanisms to therapy. Nat Med 21, 1424-1435 (2015).
24. O. Moiseeva et al., Metformin inhibits the senescence-associated secretory phenotype by interfering with IKK/NF-κB activation. Aging cell 12, 489-498 (2013).
25. L. E. Smith et al., Oxygen-induced retinopathy in the mouse. Invest Ophthalmol Vis Sci 35, 101-111 (1994).
26. J. Acosta et al., A complex secretory program orchestrated by the inflammasome controls paracrine senescence. Nature Cell Biology 15, 978-990 (2013).
27. J. Campisi, F. d'Adda di Fagagna, Cellular senescence: when bad things happen to good cells. Nat Rev Mol Cell Biol 8, 729-740 (2007).
28. Y. Okuno, A. Nakamura-Ishizu, K. Otsu, T. Suda, Y. Kubota, Pathological neoangiogenesis depends on oxidative stress regulation by ATM. Nat Med 18, 1208-1216 (2012).
29. C. Denoyelle et al., Anti-oncogenic role of the endoplasmic reticulum differentially activated by mutations in the MAPK pathway. Nat Cell Biol 8, 1053-1063 (2006).
30. A. Stahl et al., Postnatal Weight Gain Modifies Severity and Functional Outcome of Oxygen-Induced Proliferative Retinopathy. Am J Pathol.
31. A. Dorfman, O. Dembinska, S. Chemtob, P. Lachapelle, Early manifestations of postnatal hyperoxia on the retinal structure and function of the neonatal rat. Invest Ophthalmol Vis Sci 49, 458-466 (2008).
32. P. Perez Mancera, A. R. J. Young, M. Narita, Inside and out: the activities of senescence in cancer. Nature Reviews. Cancer 14, 547-558 (2014).
33. F. Binet et al., Neuronal ER Stress Impedes Myeloid-Cell-Induced Vascular Regeneration through IRE1alpha Degradation of Netrin-1. Cell Metab 17, 353-371 (2013).
34. R. Cao et al., VEGFR1-mediated pericyte ablation links VEGF and PIGF to cancer-associated retinopathy. Proc Natl Acad Sci U S A 107, 856-861 (2010).
35. J. Honek et al., Modulation of age-related insulin sensitivity by VEGF-dependent vascular plasticity in adipose tissues. Proc Natl Acad Sci U S A 111, 14906-14911 (2014).
36. C. M. Beausejour et al., Reversal of human cellular senescence: roles of the p53 and p16 pathways. EMBO J 22, 4212-4222 (2003).
37. J .-P. Coppé, P .-Y. Desprez, A. Krtolica, J. Campisi, The senescence-associated secretory phenotype: the dark side of tumor suppression. Annual review of pathology 5, 99-118 (2010).
38. J. Acosta et al., Chemokine signaling via the CXCR2 receptor reinforces senescence. Cell 133, 1006-1018 (2008).
39. T. Kuilman et al., Oncogene-induced senescence relayed by an interleukin-dependent inflammatory network. Cell 133, 1019-1031 (2008).
40. N. Wajapeyee, R. Serra, X. Zhu, M. Mahalingam, M. Green, Oncogenic BRAF induces senescence and apoptosis through pathways mediated by the secreted protein IGFBP7. Cell 132, 363-374 (2008).
41. Y. Bai et al., Effects of semaphorin 3A on retinal pigment epithelial cell activity. Investigative ophthalmology & visual science 54, 6628-6638 (2013).
42. J .-S. Joyal et al., Ischemic neurons prevent vascular regeneration of neural tissue by secreting semaphorin 3A. Blood 117, 6024-6035 (2011).
43. P. Sapieha, Eyeing central neurons in vascular growth and reparative angiogenesis. Blood 120, 2182-2194 (2012).
44. A. F. Thompson, M. E. Crowe, C. J. Lieven, L. A. Levin, Induction of Neuronal Morphology in the 661 W Cone Photoreceptor Cell Line with Staurosporine. PLOS One 10, e0145270 (2015).
45. A. Cerani et al., Neuron-derived semaphorin 3A is an early inducer of vascular permeability in diabetic retinopathy via neuropilin-1. Cell metabolism 18, 505-518 (2013).
46. F. Binet, P. Sapieha, ER Stress and Angiogenesis. Cell Metab, (2015).
47. I. Tabas, D. Ron, Integrating the mechanisms of apoptosis induced by endoplasmic reticulum stress. Nat Cell Biol 13, 184-190 (2011).
48. K. Zhang et al., The unfolded protein response transducer IRE1a prevents ER stress-induced hepatic steatosis. EMBO Journal 30, 1357-1375 (2011).
49. P. Sapieha et al., The succinate receptor GPR91 in neurons has a major role in retinal angiogenesis. Nat Med 14, 1067-1076 (2008).
50. Y. Wei et al., Nrf2 in ischemic neurons promotes retinal vascular regeneration through regulation of semaphorin 6A. Proceedings of the National Academy of Sciences of the United States of America 112, E6927-6936 (2015).
51. A. Dejda et al., Neuropilin-1 mediates myeloid cell chemoattraction and influences retinal neuroimmune crosstalk. Journal of Clinical Investigation 124, 4807-4822 (2014).
52. M. Schroder, R. J. Kaufman, The mammalian unfolded protein response. Annu Rev Biochem 74, 739-789 (2005).
53. P. Walter, D. Ron, The unfolded protein response: from stress pathway to homeostatic regulation. Science 334, 1081-1086 (2011).
54. J. Hollien, J. S. Weissman, Decay of Endoplasmic Reticulum-Localized mRNAs During the Unfolded Protein Response. Science 313, 104-107 (2006).
55. F. A. Mallette, M. F. Gaumont-Leclerc, G. Ferbeyre, The DNA damage signaling pathway is a critical mediator of oncogene-induced senescence. Genes Dev 21, 43-48 (2007).
56. O. Moiseeva, X. Deschênes Simard, M. Pollak, G. Ferbeyre, Metformin, aging and cancer. Aging 5, 330-331 (2013).
57. J. Holash et al., VEGF-Trap: a VEGF blocker with potent antitumor effects. Proc Natl Acad Sci U S A 99, 11393-11398 (2002).
58. J. S. Heier et al., Intravitreal aflibercept (VEGF trap-eye) in wet age-related macular degeneration. Ophthalmology 119, 2537-2548 (2012).
59. M. Chopp, Z. G. Zhang, Q. Jiang, Neurogenesis, angiogenesis, and MRI indices of functional recovery from stroke. Stroke 38, 827-831 (2007).
60. L. Li et al., Angiogenesis and improved cerebral blood flow in the ischemic boundary area detected by MRI after administration of sildenafil to rats with embolic stroke. Brain Res 1132, 185-192 (2007).
61. L. Hayflick, P. S. Moorhead, The serial cultivation of human diploid cell strains. Exp Cell Res 25, 585-621 (1961).
62. M. M. Edwards et al., The deletion of Math5 disrupts retinal blood vessel and glial development in mice. Exp Eye Res, (2011).
63. K. Okabe et al., Neurons limit angiogenesis by titrating VEGF in retina. Cell 159, 584-596 (2014).
64. Y. Usui et al., Neurovascular crosstalk between interneurons and capillaries is required for vision. J Clin Invest 125, 2335-2346 (2015).
65. C. D. Wiley, J. Campisi, From Ancient Pathways to Aging Cells-Connecting Metabolism and Cellular Senescence. Cell Metab 23, 1013-1021 (2016).
66. J. R. Dorr et al., Synthetic lethal metabolic targeting of cellular senescence in cancer therapy. Nature 501, 421-425 (2013).
67. J. P. Coppe, K. Kauser, J. Campisi, C. M. Beausejour, Secretion of vascular endothelial growth factor by primary human fibroblasts at senescence. J Biol Chem 281, 29568-29574 (2006).
68. F. Martinon, X. Chen, A. H. Lee, L. H. Glimcher, TLR activation of the transcription factor XBP1 regulates innate immune responses in macrophages. Nat Immunol 11, 411-418 (2010).
69. Q. Qiu et al., Toll-like receptor-mediated IRE1alpha activation as a therapeutic target for inflammatory arthritis. EMBO J 32, 2477-2490 (2013).
70. E. Check Hayden, Anti-ageing pill pushed as bona fide drug. Nature 522, 265-266 (2015).
71. B. E. Clausen, C. Burkhardt, W. Reith, R. Renkawitz, I. Forster, Conditional gene targeting in macrophages and granulocytes using LysMcre mice. Transgenic Res 8, 265-277 (1999).
72. P. Sapieha et al., Retinopathy of prematurity: understanding ischemic retinal vasculopathies at an extreme of life. J Clin Invest 120, 3022-3032 (2010).
73. A. Stahl et al., The mouse retina as an angiogenesis model. Invest Ophthalmol Vis Sci 51, 2813-2826 (2010).
74. A. Stahl et al., Computer-aided quantification of retinal neovascularization. Angiogenesis 12, 297-301 (2009).
75. G. P. Dimri et al., A biomarker that identifies senescent human cells in culture and in aging skin in vivo. Proc Natl Acad Sci U S A 92, 9363-9367 (1995).
76. T. Dull et al., A third-generation lentivirus vector with a conditional packaging system. J Virol 72, 8463-8471 (1998).
77. M. Collado et al., Tumour biology: senescence in premalignant tumours. Nature 436, 642 (2005).
78. V. Krizhanovsky et al., Senescence of activated stellate cells limits liver fibrosis. Cell 134, 657-667 (2008).
79. Antipenko A et al. (2003) Structure of the semaphorin-3A receptor binding module. Neuron 39: 589-598.
80. Shirvan A. et al., (2002). Anti-semaphorin 3A Antibodies Rescue Retinal Ganglion Cells from Cell Death following Optic Nerve Axotomy. JBC 277 (51): 49799-49807.
81. Hasan et al. (2011) Inhibition of VEGF induces cellular senescence in colorectal cancer cells. Int. J. Cancer 1;129(9) :2115-2123

1. A method of inhibiting or preventing (i) senescence of a cell or (ii) the senescence-associated secretory phenotype (SASP) of a cell comprising reducing SEMA3A level or activity.

2. The method of claim 1, wherein said method comprises contacting said cell with a SEMA3A antagonist.

3. A method of inhibiting or preventing (i) senescence of a cell or (ii) the senescence-associated secretory phenotype of a cell in a subject comprising administering to said subject an effective amount of a SEMA3A antagonist.

4. The method of claim 2 or 3, wherein said SEMA3A antagonist is (a) a SEMA3A antibody; (b) a SEMA3A antisense or shRNA; and/or (c) a soluble NRP1 polypeptide (NRP1 trap) which binds SEMA3A or functional variant or fragment thereof.

5. The method of claim 4, wherein said soluble NRP1 polypeptide is an NRP1 trap set forth in Table 2 or a functional variant or fragment thereof.

6. The method of any one of claims 1 to 5, wherein said cell is a neuron, a microglial cell, an endothelial cell, a myeloid cell, a monocyte, a macrophage or a fat tissue cell.

7. The method of any one of claims 1 to 6, wherein said cell is a terminally differentiated cell.

8. The method of any one of claims 1 to 7, wherein said senescence is paracrine senescence.

9. The method of any one of claims 1 to 8, wherein said senescence is secondary to cellular ischemia.

10. The method of any one of claims 1 to 9, wherein said method reduces IRE1a activation and the expression of Pai1, IL-6, Il-1β, TGF-b, tp53, XBP1(s) and/or Vegfa in said cells.

11. A composition for inhibiting or preventing (i) senescence of a cell or (ii) the senescence-associated secretory phenotype of a cell comprising a SEMA3A antagonist and a carrier.

12. Use of a SEMA3A antagonist for inhibiting or preventing senescence of a cell or the senescence-associated secretory phenotype of a cell.

13. The composition of claim 11 or the use of claim 12, wherein said SEMA3A antagonist is (a) a SEMA3A antibody; (b) a SEMA3A antisense or shRNA; and/or (c)(i) a soluble NRP1 polypeptide (NRP1 trap) which binds SEMA3A or functional variant or fragment thereof; or (ii) a nucleic acid encoding the polypeptide of (i) or a vector comprising said nucleic acid.

14. The composition of claim 11 or 13 or the use of claim 12 or 13, wherein said cell is a neuron, a microglial cell, an endothelial cell, a myeloid cell, a monocyte, a macrophage or a fat tissue cell.

15. The composition of claim 11 or 13 or the use of claim 12 or 13, wherein said cell is a terminally differentiated cell.

16. The composition of any one of claims 11 and 13 to 15 or the use of any one of claims 12-15, wherein said senescence is paracrine senescence.

17. The composition of any one of claims 11 and 13 to 16 or the use of any one of claims 12-16, wherein said antagonist reduces IRE1α activation and the expression of Pai1, IL-6, II-1b, TGF-b, tp53, XBP1(s) and Vegfa in said cells.

18. A SEMA3A antagonist for use in the preparation of a medicament for (i) inhibiting or preventing senescence of a cell; or (ii) the senescence-associated secretory phenotype (SASP) of a cell.

19. The SEMA3A antagonist for use of claim 18, wherein said antagonist is (a) a SEMA3A antibody; (b) a SEMA3A antisense or shRNA; and/or (c)(i) a soluble NRP1 polypeptide (NRP1 trap) or functional variant or fragment thereof or (ii) a nucleic acid encoding the polypeptide of (i) or a vector comprising said nucleic acid.

20. The method of any one of claims 1 to 10, the composition of any one of claims 11 and 13-17, the use of any one of claims 12 to 17 or the SEMA3A antagonist for use of claim 18 or 19, wherein said cell is from a subject suffering from sarcopenia, neurodegeneration, thinning of the epidermis, skin wrinkling, hair loss, chronic obstructive pulmonary disease (COPD), Idiopathic pulmonary fibrosis (IPF), atherosclerosis, osteoarthritis, osteoporosis, Parkinson's disease, intestinal bowel disease, glaucoma, intervertebral disc degeneration, brain aneurysm, aortic aneurysm, pancreatic fibrosis, cystic fibrosis, obesity, or metabolic syndrome.

21. A method of stimulating or inducing senescence of a cell or the senescence-associated secretory phenotype (SASP) of a cell comprising increasing SEMA3A level or activity.

22. A method of stimulating or inducing senescence of a cell or the senescence-associated secretory phenotype of a cell comprising contacting said cell with a SEMA3A polypeptide or functional variant or fragment thereof.

23. A method of stimulating or inducing senescence of a cell or the senescence-associated secretory phenotype of a cell of a subject comprising administering to said subject a SEMA3A polypeptide or functional variant or fragment thereof or a nucleic acid encoding said SEMA3A polypeptide or functional variant or fragment thereof for expressing said SEMA3A polypeptide.

24. The method of any one of claims 21 to 23, for improving wound healing (e.g., cutaneous wound healing).

25. The method of any one of claims 21 to 24, wherein said method increases IRE1α activation and the expression of Pai1, IL-6, II-1b, TGF-b, tp53, XBP1(s) and/or Vegfa in said cells.

26. A composition for stimulating or inducing senescence of a cell or the senescence-associated secretory phenotype of a cell comprising a SEMA3A polypeptide or functional variant or fragment thereof or a nucleic acid encoding said SEMA3A polypeptide or functional variant or fragment thereof.

27. Use of SEMA3A polypeptide or functional variant or fragment thereof for stimulating or inducing senescence of a cell or the senescence-associated secretory phenotype of a cell.

28. The composition of claim 26 or the use of claim 27, wherein said cell is from a subject having or at risk of having, liver fibrosis, pulmonary hypertension, myocardial infarction, cancer, renal fibrosis or cardiac fibrosis.

29. The composition of claim 26 or 28 or the use of claim 27 or 28, for improving wound healing.

30. A SEMA3A polypeptide or functional variant or fragment thereof for use in the preparation of a medicament for inducing senescence of a cell or the senescence-associated secretory phenotype of a cell.

31. A SEMA3A polypeptide or functional variant or fragment thereof for use in stimulating or inducing senescence of a cell or the senescence-associated secretory phenotype of a cell.

32. The SEMA3A polypeptide or functional variant or fragment thereof for use of claim 30 or 31, wherein said cell is from a subject having or at risk of having liver fibrosis, pulmonary hypertension, myocardial infarction or cardiac fibrosis.

33. The SEMA3A polypeptide or functional variant or fragment thereof of for use of claim 30 or 31 for improving wound healing.

34. A nucleic acid encoding a SEMA3A polypeptide or functional variant or fragment thereof for use in the preparation of a medicament for stimulating or inducing senescence of a cell or the senescence-associated secretory phenotype of a cell.

35. The nucleic acid of claim 34, wherein said cell is from a subject having or at risk of having liver fibrosis, pulmonary hypertension, myocardial infarction or cardiac fibrosis.

36. A vector comprising the nucleic acid of claim 35 or 36.

37. A cell comprising the vector of claim 36.

38. A method of treating or preventing a vascular eye disease or disorder comprising administering a SASP inhibitor to a subject, wherein said SASP inhibitor (i) an inhibitor of IRE1α expression (ii) an inhibitor of IRE1α RNAse activity; (ii) a biguanide compound; or (iii) an mTOr inhibitor.

39. The method of claim 38, wherein said vascular eye disease or disorder is diabetic retinopathy, retinopathy of prematurity, ischemic retinopathy, hypertensive retinopathy, drug-induced retinal vasculopathy, diabetic macular edema, age-related macular degeneration, juvenile macular degeneration, retinal neovascularisation, central retinal vein occlusion, branched retinal vein occlusion, choroidal neovascularization, polypoidal choroidal vasculopathy, physical injury to the eye, glaucoma, rhegmatogenous retinal detachment (RRD), retinal vasculitis, retinal macroaneurysm, retinal microaneurysm, Fuch's dystrophy, ischemic optic neuropathy, macular telangiectasia, optic neuritis, usher syndrome, retinitis pigmentosa, uveitis, ischemic optic neuropathy (ION) or stangardt disease.

40. The method of claim 39, wherein said vascular eye disease or disorder is diabetic retinopathy, retinopathy of prematurity, diabetic macular edema, age-related macular edema, retinal neovascularization, central retinal vein occlusion, branched retinal vein occlusion or choroidal neovascularization.

41. The method of claim 39, wherein said vascular eye disease or disorder is diabeticretinopathy, retinopathy of prematurity, Dry (atrophic) Age-related Macular Degeneration, wet (exudative) Age-related Macular Degeneration, Branch Retinal Vein Occlusion, or Macular Talacgiectasia.

42. A method of inhibiting retinal angiogenesis comprising administering a SASP inhibitor to a subject, wherein said SASP inhibitor is (i) an inhibitor of IRE1a expression (ii) an inhibitor of IRE1α RNAse activity; (iii) a biguanide compound; or (iv) an mTOr inhibitor.

43. The method of claim 42, wherein said retinal angiogenesis is secondary to cellular ischemia.

44. The method of any one of claims 38 to 43, wherein the subject has been diagnosed with diabetic retinopathy, retinopathy of prematurity, diabetic macular edema, age-related macular degeneration, retinal neovascularisation, central retinal vein occlusion, branched retinal vein occlusion, choroidal neovascularization, polypoidal choroidal vasculopathy, Macular Talacgiectasia.

45. A method of promoting ocular vascular repair and/or reducing ocular ischemia comprising administering a SASP inhibitor to a subject, wherein said SASP inhibitor is (i) an inhibitor of IRE1α expression (ii) an inhibitor of IRE1α RNAse activity; (iii) a biguanide compound; or (iv) an mTOr inhibitor.

46. A method of preventing or reducing ocular cellular senescence comprising administering a SASP inhibitor to a subject, wherein said SASP inhibitor (i) an inhibitor of IRE1α expression (ii) an inhibitor of IRE1α RNAse activity; (iii) a biguanide compound; or (iv) an mTOr inhibitor.

47. The method of any one of claims 38 to 46, wherein said administration is topical or local ocular administration.

48. The method of 47, wherein said local ocular administration is subconjunctival (sub-tenons), intravitreal, retrobulbar, posterior juxtascleral or intracameral administration.

49. The method of 48, wherein said local ocular administration is intravitreal administration.

50. The method of any one of claims 38 to 49, wherein said SASP inhibitor is a biguanide compound.

51. The method of claim 50, wherein said biguanide compound is metformin, phenformin, buformin, proguanil, chlorproguanil, Synthalin A or Synthalin B.

52. The method of claim 51, wherein said biguanide compound is metformin.

53. An ophthalmic composition comprising a biguanide compound and a suitable pharmaceutical carrier.

54. The ophthalmic composition of claim 53, wherein said biguanide compound is (i) metformin, (ii) phenformin; (iii) buformin; (iv) proguanil; (v) chlorproguanil; (vi) Synthalin A; (vii) Synthalin B or (iv) any combination thereof.

55. The ophthalmic composition of claim 54, wherein said biguanide compound is metformin.

56. The ophthalmic composition of any one of claims 53 to 55, for treating or preventing a vascular eye disease or disorder.

57. The ophthalmic composition of any one of claims 53 to 55, for inhibiting retinal angiogenesis.

58. The ophthalmic composition of any one of claims 53 to 55, for promoting ocular vascular repair and/or reducing ocular ischemia.

59. A composition comprising a SASP inhibitor, for use in treating or preventing a vascular eye disease or disorder, wherein said SASP inhibitor is (i) an inhibitor of IRE1a expression; (ii) an inhibitor of IRE1a RNAse activity; or (iii) an mTOr inhibitor.

60. The composition of claim 59, wherein said vascular eye disease or disorder is diabetic retinopathy, retinopathy of prematurity, ischemic retinopathy, hypertensive retinopathy, drug-induced retinal vasculopathy, diabetic macular edema, age-related macular degeneration, juvenile macular degeneration, retinal neovascularisation, central retinal vein occlusion, branched retinal vein occlusion, choroidal neovascularization, polypoidal choroidal vasculopathy, physical injury to the eye, glaucoma, rhegmatogenous retinal detachment (RRD), retinal vasculitis, retinal macroaneurysm, retinal microaneurysm, Fuch's dystrophy, ischemic optic neuropathy, macular telangiectasia, optic neuritis, usher syndrome, retinitis pigmentosa, uveitis, ischemic optic neuropathy (ION) or stangardt disease.

61. A composition comprising a SASP inhibitor, for use in inhibiting retinal angiogenesis, wherein said SASP inhibitor is (i) an inhibitor of IRE1α expression; (ii) an inhibitor of IRE1α RNAse activity; or (iii) an mTOr inhibitor.

62. A composition comprising a SASP inhibitor, for use in promoting ocular vascular repair and/or reducing ocular ischemia, wherein said SASP inhibitor is (i) an inhibitor of IRE1α expression; (ii) an inhibitor of IRE1α RNAse activity; or (iii) an mTOr inhibitor.

63. A composition comprising a SASP inhibitor, for use in preventing or reducing ocular cellular senescence, wherein said SASP inhibitor is (i) an inhibitor of IRE1α expression; (ii) an inhibitor of IRE1α RNAse activity; or (iii) an mTOr inhibitor.

64. The composition of any one of claims 59 to 63, wherein said composition is an ophthalmic composition.

65. Use of a composition as defined in any one of claims 53 to 55, 59 and 60, for treating or preventing a vascular eye disease or disorder.

66. Use of a composition as defined in any one of claims 53 to 55 for (i) inhibiting retinal angiogenesis; (ii) inhibiting pathological retinal neovascularization; (iii) promoting ocular vascular repair; (iv) reducing ocular ischemia; and/or (v) preventing or reducing ocular cellular senescence.

67. A composition as defined in any one of claims 53 to 55, for use in the preparation of a medicament for treating or preventing a vascular eye disease or disorder.

68. A composition as defined in any one of claims 53 to 55, for use in the preparation of a medicament for (i) inhibiting retinal angiogenesis; (ii) promoting ocular vascular repair and/or reducing ocular ischemia; and/or (iii) preventing or reducing ocular cellular senescence.

69. Use of a composition comprising a SASP inhibitor for (i) inhibiting retinal angiogenesis; (ii) inhibiting pathological retinal neovascularization; (iii) promoting ocular vascular repair; (iv) reducing ocular ischemia; and/or (v) preventing or reducing ocular cellular senescence, wherein said SASP inhibitor is.

70. A composition comprising a SASP inhibitor for use in the preparation of a medicament for treating or preventing a vascular eye disease or disorder, wherein said SASP inhibitor is (i) an inhibitor of IRE1α expression; (ii) an inhibitor of IRE1α RNAse activity; or (iii) an mTOr inhibitor.

71. A composition comprising a SASP inhibitor for use in the preparation of a medicament for (i) inhibiting retinal angiogenesis (pathological retinal neovascularization); (ii); promoting ocular vascular repair; (iii) reducing ocular ischemia; and/or (iv) preventing or reducing ocular cellular senescence, is (i) an inhibitor of IRE1α expression; (ii) an inhibitor of IRE1a RNAse activity; or (iii) an mTOr inhibitor.

72. A method of inhibiting or preventing (i) senescence of a cell or (ii) the senescence-associated secretory phenotype of a cell comprising reducing IRE1α expression or activity.

73. The method of claim 72, wherein said activity comprises IRE1α ribonuclease activity.

74. The method of claim 72 or 73, wherein said method comprises contacting said cell with an IRE1α inhibitor.

75. A method of inhibiting or preventing (i) senescence of a cell or (ii) the senescence-associated secretory phenotype of a cell of a subject comprising administering to said subject an IRE1α inhibitor.

76. The method of any one of claims 72 to 75, wherein said inhibitor is an antisens or shRNA against IRE1α, 4u8c, bortezomib, N-[(2-Hydroxy-1-naphthalenyl)methylene]-2-thiophenesulfonamide (STF-083010), or MKC-3946.

77. The method of any one of claims 72 to 76, wherein said cell is a terminally differentiated cell.

78. The method of claim 77, wherein said cell is a neuron or a microglial cell.

79. The method of any one of claims 72 to 78, wherein said senescence is paracrine senescence.

80. The method of any one of claims 72 to 79, wherein said SASP is secondary to cellular ischemia.

81. The method of any one of claims 72 to 80, wherein said method reduces IRE1α activation; SA-β-gal activity; and/or the expression of Pai1, IL-6, II-1b, TGF-b, tp53, XBP1(s) and/or Vegfa in said cells.

82. A composition for inhibiting or preventing (i) senescence of a cell or (ii) the senescence-associated secretory phenotype of a cell comprising an IRE1α inhibitor.

83. Use of an IRE1α inhibitor for inhibiting or preventing (i) senescence of a cell or (ii) the senescence-associated secretory phenotype of a cell.

84. The composition of claim 82 or the use of claim 83, wherein said IRE1α inhibitor is an antisens or shRNA against IRE1α, 4u8c, bortezomib, N-[(2-Hydroxy-1-naphthalenyl)methylene]-2-thiophenesulfonamide (STF-083010), or MKC-3946.

85. The composition of claim 82 or 84 or the use of claim 83 or 84, wherein said cell is a terminally differentiated cell.

86. The composition or use of claim 85, wherein said cell is a neuron or a microglial cell.

87. The composition of any one of claims 82 and 84 to 86 or the use of any one of claims 83 to 86, wherein said senescence is paracrine senescence.

88. The composition of any one of claims 82 and 84 to 87 or the use of any one of claims 83 to 87, wherein said SASP is secondary to cellular ischemia.

89. The composition of any one of claims 82 and 84 to 88 or the use of any one of claims 83 to 88, wherein said inhibitor reduces IRE1α activation; SA-β-gal activity; and/or the expression of Pai1, IL-6, Il-1b, TGF-b, tp53, XBP1(s) and/or Vegfa in said cells.

90. An IRE1α inhibitor for use in the preparation of a medicament for inhibiting or preventing (i) senescence of a cell or (ii) the senescence-associated secretory phenotype of a cell.

91. The IRE1α inhibitor for use of claim 90, wherein said inhibitor is an antisens or shRNA against IRE1α, bortezomib, N-[(2-Hydroxy-1-naphthalenyl)methylene]-2-thiophenesulfonamide (STF-083010), or MKC-3946.

92. The method of any one of claims 72 to 81, the composition of any one of claims 82 and 84 to 89, the use of any one of claims 83 to 89 or the IRE1α inhibitor for use of claim 90 or 91, wherein said cell is from a subject suffering or at risk of suffering from sarcopenia, neurodegeneration, thinning of the epidermis, skin wrinkling, hair loss, chronic obstructive pulmonary disease (COPD), idiopathic pulmonary fibrosis (IPF), atherosclerosis, osteoarthritis, osteoporosis, Parkinson's disease, intestinal bowel disease, glaucoma, intervertebral disc degeneration, brain aneurysm, aortic aneurysm, pancreatic fibrosis or cystic fibrosis.

93. The method of any one of claims 72 to 81 and 92, the composition of any one of claims 82 and 84 to 89 and 92, the use of any one of claims 83 to 89 and 92 or the IRE1a inhibitor for use of any one of claims 90 to 92, wherein said cell is from a subject which is has undergone cancer treatment or is undergoing cancer treatment.

94. The method of any one of claims 72 to 81, 92 and 93, the composition of any one of claims 82 and 84 to 89, 92 and 93, the use of any one of claims 83 to 89, 92 and 93, or the IRE1α inhibitor for use of any one of claims 90 to 93, wherein said cell is not a retinal cell and wherein said senescence is not associated with a retinal vascular disease.

95. A method of stimulating or inducing senescence of a cell or the senescence-associated secretory phenotype (SASP) of a cell comprising increasing IRE1α level or activity.

96. The method of claim 95, wherein said method comprises contacting said cell with a compound which increases IRE1α level or activity.

97. A method of stimulating or inducing senescence of a cell or the senescence-associated secretory phenotype of a cell of a subject comprising administering to said subject an effective amount of a compound which increases IRE1α level or activity.

98. The method of claim 96 or 97, wherein said compound is Apy29 or Sunitinib.

99. The method of any one of claims 96 to 98, wherein said cell is from a subject having or at risk of having, liver fibrosis, renal fibrosis, pulmonary hypertension, myocardial infarction or cardiac fibrosis.

100. The method of any one of claims 96 to 99, for improving wound healing.

101. The method of any one of claims 96 to 100, wherein said method increases IRE1α activation; SA-β-gal activity; and/or the expression of Pai1, IL-6, II-1b, TGF-b, tp53, XBP1(s) and/or Vegfa in said cells.

102. A composition for stimulating or inducing senescence of a cell or the senescence-associated secretory phenotype in a cell comprising a compound which increases IRE1α level or activity.

103. The composition of claim 102, wherein said compound is Apy29 or Sunitinib.

104. Use of a compound increases IRE1α level or activity for stimulating or inducing senescence of a cell or the senescence-associated secretory phenotype in a cell.

105. The composition of claim 102 or 103 or the use of claim 67, wherein said cell is from a subject having or at risk of having, liver fibrosis, renal fibrosis, pulmonary hypertension, myocardial infarction or cardiac fibrosis.

106. The composition of any one of claim 102, 103 or 105 or the use of claim 67 or 68, for improving wound healing.

107. A compound which increases IRE1α level or activity for use in the preparation of a medicament for inducing senescence of a cell or the senescence-associated secretory phenotype in a cell.

108. A compound which increases IRE1α for use in stimulating or inducing senescence of a cell or the senescence-associated secretory phenotype in a cell.

109. The compound for use of claim 107 or 108, wherein said cell is from a subject having or at risk of having liver fibrosis, renal fibrosis, pulmonary hypertension, myocardial infarction or cardiac fibrosis.

110. The compound for use of any one of claims 108 to 109 for improving wound healing.

111. A method of altering a lipid parameter in a subject, said method comprising administering to the subject: (a) a soluble NRP1 polypeptide or variant or fragment thereof; (b) an NRP1 antibody; or (c) a composition comprising (a) and/or (b) together with a pharmaceutically acceptable carrier,
wherein said altering of a lipid parameter is (a) a decrease in total cholesterol level; (b) a decrease in non-HDL cholesterol level; (c) a decrease in triglycerides level; (d) a decrease in the ratio of total cholesterol:HDL cholesterol; (e) a decrease in circulating free fatty acid; (f) an increase in HDL cholesterol or (f) any combination of (a) to (e).

112. A method for preventing or treating a disease or condition associated with fat accumulation in a subject, said method comprising administering to the subject: (a) a soluble NRP1 polypeptide or functional variant or fragment thereof; (b) an NRP1 antibody; or (c) a composition comprising (a) and/or (b) together with a pharmaceutically acceptable carrier.

113. The method of claim 112, wherein said disease or condition associated with fat accumulation is high body mass index (BMI); obesity; metabolic syndrome; NAFLD; a cardiovascular disease (CVD); hypertension and/or Type II Diabetes mellitus (TIIDM).

114. The method of claim 113, wherein said cardiovascular disease is congestive heart failure, hypercholesterolemia and/or atherosclerosis.

115. A method for altering a body composition parameter in a subject comprising administering to the subject (a) a soluble NRP1 polypeptide or functional variant or fragment thereof; (b) an NRP1 antibody; or (c) a composition comprising (a) and/or (b) together with a pharmaceutically acceptable carrier, wherein said body composition parameter is visceral fat area (VFA), body mass index (BMI), waist to hip ratio (WHR); waist-to-height ratio (WHeR), waist circumference (WC); arm circumference (AC), conicity index, per cent body fat (PBF), triceps skin fold, subscapular skin fold, white adipose tissue (WAT) level; and or brown adipose (BAT) tissue level.

116. The method of any one of claims 111 to 115, wherein said soluble NRP1 polypeptide or fragment thereof comprises or consists of an NRP1 polypeptide trap described in Table 2 or set forth in FIG. 17 or 18.

117. The method of any one of claims 111 to 116, wherein said soluble NRP1 polypeptide or functional variant or fragment thereof is administered systemically.

118. A composition comprising (a) a soluble NRP1 polypeptide or functional variant or fragment thereof; (b) an NRP1 antibody; or (c) a composition comprising (a) and/or (b), together with a pharmaceutically acceptable carrier, for altering a lipid parameter in a subject,
wherein said alteration of a lipid parameter is (a) a decrease in total cholesterol level; (b) a decrease in non-HDL cholesterol level; (c) a decrease in triglycerides level; (d) a decrease in the ratio of total cholesterol:HDL cholesterol; (e) a decrease in circulating free fatty acid; (f) an increase in HDL cholesterol or (f) any combination of (a) to (e).

119. A composition comprising (a) a soluble NRP1 polypeptide or functional variant or fragment thereof; (b) an NRP1 antibody; or (c) a composition comprising (a) and/or (b), together with a pharmaceutically acceptable carrier, for preventing or treating a disease or condition associated with fat accumulation in a subject.

120. The composition of claim 119, wherein said disease or condition associated with fat accumulation is high body mass index (BMI); obesity; metabolic syndrome; NAFLD; a cardiovascular disease (CVD); hypertension and/or Type II Diabetes mellitus (TIIDM).

121. The composition of claim 120, wherein said cardiovascular disease is congestive heart failure, hypercholesterolemia and/or atherosclerosis.

122. A composition comprising (a) a soluble NRP1 polypeptide or functional variant or fragment thereof; (b) an NRP1 antibody; or (c) a composition comprising (a) and/or (b), together with a pharmaceutically acceptable carrier for altering a body composition parameter in a subject,
wherein said body composition parameter is visceral fat area (VFA), body mass index (BMI), waist to hip ratio (WHR); waist-to-height ratio (WHeR), waist circumference (WC); arm circumference (AC), conicity index, per cent body fat (PBF), triceps skin fold, subscapular skin fold, white adipose tissue (WAT) level; and or brown adipose (BAT) tissue level.

123. The composition of any one of claims 118 to 122, wherein said soluble NRP1 polypeptide comprises or consists of an NRP1 polypeptide trap described in Table 2 or set forth in FIG. 17 or 18.

124. The composition of any one of claims 118 to 123, wherein said soluble NRP1 polypeptide or functional variant or fragment thereof is for systemic administration.

125. Use of (a) a soluble NRP1 polypeptide or functional variant or fragment thereof; (b) an NRP1 antibody; or (c) a composition comprising (a) and/or (b) together with a pharmaceutically acceptable carrier; for altering a lipid parameter in a subject,
wherein said alteration of a lipid parameter is (a) a decrease in total cholesterol level; (b) a decrease in non-HDL cholesterol level; (c) a decrease in triglycerides level; (d) a decrease in the ratio of total cholesterol:HDL cholesterol; (e) a decrease in circulating free fatty acid; (f) an increase in HDL cholesterol or (f) any combination of (a) to (e).

126. Use of a soluble NRP1 polypeptide or functional variant or fragment thereof; (b) an NRP1 antibody; or (c) a composition comprising (a) and/or (b), together with a pharmaceutically acceptable carrier, for preventing or treating a disease or condition associated with fat accumulation in a subject.

127. The use of claim 126, wherein said disease or condition associated with fat accumulation is high body mass index (BMI); obesity; metabolic syndrome; NAFLD; a cardiovascular disease (CVD); hypertension and/or Type II Diabetes mellitus (TIIDM).

128. The use of claim 127, wherein said cardiovascular disease is congestive heart failure, hypercholesterolemia and/or atherosclerosis.

129. A use of (a) a soluble NRP1 polypeptide or functional variant or fragment thereof; (b) an NRP1 antibody; or (c) a composition comprising (a) and/or (b), together with a pharmaceutically acceptable carrier for altering a body composition parameter in a subject,
wherein said body composition parameter is visceral fat area (VFA), body mass index (BMI), waist to hip ratio (WHR); waist-to-height ratio (WHeR), waist circumference (WC); arm circumference (AC), conicity index, per cent body fat (PBF), triceps skin fold, subscapular skin fold, white adipose tissue (WAT) level; and or brown adipose (BAT) tissue level.

130. The use of any one of claims 125 to 129, wherein said soluble NRP1 polypeptide or functional variant or fragment thereof comprises or consists of an NRP1 polypeptide trap described in Table 2 or set forth in FIG. 7 or 9A.

131. The use of any one of claims 125 to 130, wherein said soluble NRP1 polypeptide or functional variant or fragment thereof is for systemic administration.

132. Use of (a) a soluble NRP1 polypeptide or functional variant or fragment thereof; (b) an NRP1 antibody; or (c) a composition comprising (a) and/or (b) together with a pharmaceutically acceptable carrier; for the preparation of a medicament for altering a lipid parameter in a subject,
wherein said altering of a lipid parameter is (a) a decrease in total cholesterol level; (b) a decrease in non-HDL cholesterol level; (c) a decrease in triglycerides level; (d) a decrease in the ratio of total cholesterol:HDL cholesterol; (e) a decrease in circulating free fatty acid; (f) an increase in HDL cholesterol or (f) any combination of (a) to (e).

133. Use of a soluble NRP1 polypeptide or functional variant or fragment thereof; (b) an NRP1 antibody; or (c) a composition comprising (a) and/or (b), together with a pharmaceutically acceptable carrier, for the preparation of a medicament for preventing or treating a disease or condition associated with fat accumulation in a subject.

134. The use of claim 133, wherein said disease or condition associated with fat accumulation is high body mass index (BMI); obesity; metabolic syndrome; NAFLD; a cardiovascular disease (CVD); hypertension and/or Type II Diabetes mellitus (TIIDM).

135. The use of claim 134, wherein said cardiovascular disease is congestive heart failure, hypercholesterolemia and/or atherosclerosis.

136. A use of (a) a soluble NRP1 polypeptide or functional variant or fragment thereof; (b) an NRP1 antibody; or (c) a composition comprising (a) and/or (b), together with a pharmaceutically acceptable carrier for the preparation of a medicament for altering a body composition parameter in a subject, wherein said body composition parameter is visceral fat area (VFA), body mass index (BMI), waist to hip ratio (WHR); waist-to-height ratio (WHeR), waist circumference (WC); arm circumference (AC), conicity index, per cent body fat (PBF), triceps skin fold, subscapular skin fold, white adipose tissue (WAT) level; and or brown adipose (BAT) tissue level.

137. The use of any one of claims 132 to 136, wherein said soluble NRP1 polypeptide comprises or consists of an NRP1 polypeptide trap described in Table 2 or set forth in FIG. 17 or 18.

138. The use of any one of claims 132 to 137, wherein said medicament is for systemic administration.

139. A soluble NRP1 polypeptide set for in SEQ ID NO: 52, 54, 58, 60, 62, 64, 66, or 76 or a functional variant or fragment thereof.

140. A nucleic acid encoding the soluble NRP1 polypeptide a functional variant or fragment of claim 139.

141. A vector comprising the nucleic acid of claim 140.

142. The vector of claim 141, which is a viral vector (preferably a lentiviral or adenoviral vector).

143. A host cell comprising the nucleic acid of claim 140 or the vector of claim 142 or 142.
